(19) United States
(12) Patent Application Publication
(10) Pub. No.: US 2021/0309993 A1
(43) Pub. Date: Oct. 7, 2021

(54) RANDOMIZED PEPTIDE LIBRARIES PRESENTED BY HUMAN LEUKOCYTE ANTIGENS

(71) Applicant: 3T BIOSCIENCES, INC., SOUTH SAN FRANCISCO, CA (US)

(72) Inventors: MARVIN GEE, SAN FRANCISCO, CA (US); LEAH SIBENER, SAN FRANCISCO, CA (US)

(73) Assignee: 3T BIOSCIENCES, INC., SOUTH SAN FRANCISCO, CA (US)

(21) Appl. No.: 17/272,088

(22) PCT Filed: Aug. 30, 2019
(86) PCT No.: PCT/US2019/049205

§ 371 Date: Feb. 26, 2021

Related U.S. Application Data
(60) Provisional application No. 62/726,060, filed on Aug. 31, 2018.

(51) Int. Cl. C12N 15/10; C07K 14/74

(52) U.S. Cl. CPC C12N 15/1037; C07K 14/70539; C12N 15/1086

(57) ABSTRACT

Described herein is an antigen screening library comprising a plurality of Human Leukocyte Antigen (HLA)-antigen polypeptide complexes, the HLA-antigen polypeptide complexes comprising: (a) an HLA polypeptide; (b) a randomized antigen polypeptide comprising an amino acid sequence set forth in any one of SEQ ID NOs: 1 to 209, wherein the randomized antigen polypeptide is selected to specifically bind to peptide binding cleft of the HLA polypeptide; and (c) a β2 microglobulin polypeptide. These libraries can be used to determine antigenic polypeptides capable of interacting and stimulating a selected T cell receptor (TCR).

### CROSS-REFERENCE TO RELATED APPLICATIONS

The present application claims the benefit of U.S. Provisional Application No. 62/726,060, filed Aug. 31, 2018, the contents of which are hereby incorporated by reference in their entirety.

### BACKGROUND

T cells are vital to the adaptive immune response, having roles in response to infection and cancer. T cells recognize proteins derived from foreign pathogens as well as self, such as in cases of autoimmunity. Fragments of these proteins (e.g., peptides) are presented by human leukocyte antigen (HLA) molecules and recognized by the T cell via the T cell receptor (TCR).

Major histocompatibility class (MEW) I HLA molecules display peptides generated largely from processing endogenous antigens produced by the cell, such as self-antigens, but also foreign intracellular antigens such as peptides derived from viral proteins, into smaller peptides. Once a peptide is bound into the HLA peptide binding cleft, MHC class I HLA molecules interact with and stimulate CD8+ cytotoxic T cells. MHC class I has 3 main loci A, B, and C, with each loci divided into many alleles. Alleles refer to the DNA sequence of a gene at the given locus and is usually denoted by at least a four-digit number (e.g., A*24:02) the first letter designating the locus, a first number defining an allele group (or type) and the second number defining a specific protein within the allele group. A second and third number can be appended indicating silent coding variants and non-coding variants respectively.

Upon recognition of a specific peptide-HLA complex (pHLA), the T cell becomes activated and can (1) become cytotoxic, (2) secrete cytokines, and/or (3) recruit other immune cells. This complex interaction between a foreign or self-peptide, HLA molecule, and TCR is central to identifying how the immune system responds to recognized pathogens at the molecular level. One of the greatest difficulties in this complex interaction during an immune response is understanding the specificities of TCRs in terms of the identity of the peptides that are recognized. New methods of identifying TCRs and the pHLAs that they recognize are needed.

### SUMMARY

Provided herein in some embodiments are antigen screening libraries comprising a plurality of Human Leukocyte Antigen (HLA)-antigen polypeptide complexes, the HLA-antigen polypeptide complexes comprising (a) an HLA polypeptide, the HLA polypeptide comprising a peptide binding cleft, (b) a randomized antigen polypeptide comprising an amino acid sequence set forth in any one of SEQ ID NOs: 1 to 209, wherein the randomized antigen polypeptide specifically binds to the peptide binding cleft of the HLA polypeptide, and (c) a Beta-2 (β2) microglobulin polypeptide.

In some embodiments, the plurality of HLA-antigen complexes comprises an HLA polypeptide selected from the list consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises at least five, ten, fifteen, twenty, or twenty-five different HLA polypeptides selected from the list consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises all of A3, A11, A23, A24, A26, A30, A31, A33, A68, B7, B8, B15, B27, B40, B44, B51, B53, C1, C2, C3, C4, C5, C6, C7, C8, and E HLA polypeptides.

In some embodiments, the plurality of HLA-antigen complexes comprises an HLA polypeptide comprising an amino acid sequence at least 87.5%, 90%, 95%, 97%, 98%, 99%, or 100% identical to an amino acid sequence set forth in any one of SEQ ID NOs: 427 to 455.

In some embodiments, the plurality of the HLA-antigen polypeptide complexes comprises at least about 10⁵ different HLA-antigen polypeptide complexes comprising at least about 10⁵ different randomized antigen polypeptides.

In some embodiments, the HLA polypeptide, the randomized antigen polypeptide, and the β2-microglobulin polypeptide comprise a single polypeptide. In some embodiments, the single polypeptide further comprises a first flexible polypeptide linker and a second flexible polypeptide linker. In some embodiments, the randomized antigen polypeptide is N-terminal to the HLA polypeptide on the single polypeptide, and the HLA polypeptide is N-terminal to the β2-microglobulin polypeptide on the single polypeptide. In these embodiments, the first flexible polypeptide linker separates the HLA polypeptide from the randomized antigen polypeptide, and a second flexible polypeptide linker separates the HLA polypeptide from the β2-microglobulin polypeptide. In some embodiments, the randomized antigen polypeptide is C-terminal to the HLA polypeptide on the single polypeptide, and the HLA polypeptide is N-terminal to the β2-microglobulin polypeptide on the single polypeptide. In these embodiments, the first flexible polypeptide linker separates the HLA polypeptide from the randomized antigen polypeptide, and a second flexible polypeptide linker separates the HLA polypeptide from the β2-microglobulin polypeptide. In some embodiments, the randomized antigen polypeptide is N-terminal to the HLA polypeptide on the single polypeptide, and the HLA polypeptide is C-terminal to the β2-microglobulin polypeptide on the single polypeptide. In these embodiments, the first flexible polypeptide linker separates the randomized antigen polypeptide from the β2-microglobulin polypeptide, and a second flexible polypeptide linker separates the β2-microglobulin polypeptide from the HLA polypeptide. In some embodiments, the randomized antigen polypeptide is C-terminal to the HLA polypeptide on the single polypeptide, and the HLA polypeptide is C-terminal to the β2-microglobulin polypeptide on the single polypeptide. In these embodiments, the first flexible polypeptide linker separates the HLA polypeptide from the β2-microglobulin polypeptide, and a second flexible polypeptide linker separates the randomized antigen polypeptide from the HLA polypeptide. In some embodiments, the β2-microglobulin polypeptide is C-terminal to the HLA polypeptide on the single polypeptide, and the HLA polypeptide is N-terminal to the randomized antigen polypeptide on the single polypeptide. In these embodiments, the first flexible polypeptide linker separates the HLA polypeptide from the randomized antigen polypeptide, and a second flexible polypeptide linker separates the randomized antigen polypeptide from the β2-microglobulin polypeptide. In some embodiments, the randomized antigen polypeptide is C-terminal to the β2-microglobulin on the single polypeptide, and the HLA polypeptide is C-terminal to the randomized antigen polypeptide on the single polypeptide. In these embodiments, the first flexible polypeptide linker separates the β2-microglobulin polypeptide from the randomized antigen polypeptide, and a second flexible polypeptide linker separates the randomized antigen polypeptide from the HLA polypeptide.

In some embodiments, each of the HLA-antigen complexes of the plurality of the HLA-antigen complexes do not comprise an epitope tag. In some embodiments, at least one of the HLA-antigen complexes of the plurality of HLA-antigen complexes comprise an epitope tag. In some embodiments, at least one of the HLA-antigen complexes of the plurality of HLA-antigen complexes does not comprise an epitope tag and at least one of the HLA-antigen complexes of the plurality of HLA-antigen complexes does comprise an epitope tag. In some embodiments, the epitope tag comprises a FLAG tag, a c-MYC tag, a HIS-tag, a hemagglutinin (HA) tag, a VSVg tag, or a V5 tag.

In some embodiments, the HLA-antigen complexes each comprise a membrane tethering domain. In some embodiments, the membrane tethering domain comprises Aga2. In some embodiments, the antigen screening library is expressed on a plurality of cells.

In some embodiments, the plurality of cells are a plurality of yeast cells. In some embodiments, the plurality of yeast cells are a plurality of yeast cells of the EBY100 strain of Saccharomyces cerevisiae.

In some embodiments, each cell of the plurality of cells expresses a specific HLA-antigen complex.

Provided herein in some embodiments are antigen screening libraries comprising a plurality of Human Leukocyte Antigen (HLA)-antigen polypeptide complexes, the HLA-antigen polypeptide complexes comprising an HLA polypeptide, the HLA polypeptide comprising a peptide binding cleft, and a randomized antigen polypeptide comprising an amino acid sequence set forth in any one of SEQ ID NOs: 1 to 209, wherein the randomized antigen polypeptide specifically binds to the peptide binding cleft of the HLA polypeptide.

In some embodiments, the plurality of HLA-antigen complexes comprises an HLA polypeptide selected from the list consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises at least five, ten, fifteen, twenty, or twenty-five different HLA polypeptides selected from the list consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises all of A3, A11, A23, A24, A26, A30, A31, A33, A68, B7, B8, B15, B27, B40, B44, B51, B53, C1, C2, C3, C4, C5, C6, C7, C8, and E HLA polypeptides.

In some embodiments, the plurality of HLA-antigen complexes comprises an HLA polypeptide comprising an amino acid sequence at least 87.5%, 90%, 95%, 97%, 98%, 99%, or 100% identical to an amino acid sequence set forth in any one of SEQ ID NOs: 427 to 455.

In some embodiments, the plurality of the HLA-antigen polypeptide complexes comprises at least about 10⁵ different HLA-antigen polypeptide complexes comprising at least about 10⁵ different randomized antigen polypeptides.

In some embodiments, the HLA polypeptide, the randomized antigen polypeptide, and the β2-microglobulin polypeptide comprise a single polypeptide. In some embodiments, the single polypeptide further comprises a first flexible polypeptide linker separating the HLA polypeptide from the randomized antigen polypeptide. In certain of these embodiments, the randomized antigen polypeptide is N-terminal to the HLA polypeptide on the single polypeptide. In certain of these embodiments, the randomized antigen polypeptide is C-terminal to the HLA polypeptide on the single polypeptide.

In some embodiments, each of the HLA-antigen complexes of the plurality of the HLA-antigen complexes do not comprise an epitope tag. In some embodiments, at least one of the HLA-antigen complexes of the plurality of HLA-antigen complexes comprise an epitope tag. In some embodiments, at least one of the HLA-antigen complexes of the plurality of HLA-antigen complexes does not comprise an epitope tag and at least one of the HLA-antigen complexes of the plurality of HLA-antigen complexes does comprise an epitope tag. In some embodiments, the epitope tag comprises a FLAG tag, a c-MYC tag, a HIS-tag, a hemagglutinin (HA) tag, a VSVg tag, or a V5 tag.

In some embodiments, the HLA-antigen complexes each comprise a membrane tethering domain. In some embodiments, the membrane tethering domain comprises Aga2. In some embodiments, the antigen screening library is expressed on a plurality of cells.

In some embodiments, the plurality of cells are a plurality of yeast cells. In some embodiments, the plurality of yeast cells are a plurality of yeast cells of the EBY100 strain of Saccharomyces cerevisiae.

In some embodiments, each cell of the plurality of cells expresses a specific HLA-antigen complex.

Provided herein in some embodiments are antigen screening libraries comprising a plurality of antigen polypeptide-Beta-2 (β2) microglobulin polypeptide complexes, the antigen polypeptide-Beta-2 (β2) microglobulin polypeptide complexes. In these embodiments, the antigen screening libraries further comprise a randomized antigen polypeptide comprising an amino acid sequence set forth in any one of SEQ ID NOs: 1 to 209, wherein the randomized antigen polypeptide specifically binds to the peptide binding cleft of the HLA polypeptide; and a Beta-2 (β2) microglobulin polypeptide. In these embodiments, the antigen screening libraries also further comprise a plurality of HLA polypeptides constitutively expressed by one or more yeast cells and comprising a peptide binding cleft.

In some embodiments, the plurality of HLA-antigen complexes comprises an HLA polypeptide selected from the list consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises at least five, ten, fifteen, twenty, or twenty-five different HLA polypeptides selected from the list consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises all of A3, A11, A23, A24, A26, A30, A31, A33, A68, B7, B8, B15, B27, B40, B44, B51, B53, C1, C2, C3, C4, C5, C6, C7, C8, and E HLA polypeptides.

In some embodiments, the plurality of HLA-antigen complexes comprises an HLA polypeptide comprising an amino acid sequence at least 87.5%, 90%, 95%, 97%, 98%, 99%, or 100% identical to an amino acid sequence set forth in any one of SEQ ID NOs: 427 to 455.

In some embodiments, the plurality of the antigen polypeptide-Beta-2 (β2) microglobulin polypeptide complexes comprises at least about 10⁵ different antigen polypeptide-Beta-2 (β2) microglobulin polypeptide complexes comprising at least about 10⁵ different randomized antigen polypeptides.

In some embodiments, the randomized antigen polypeptide and the β2-microglobulin polypeptide comprise a single polypeptide. In some embodiments, the single polypeptide further comprises a first flexible polypeptide linker. In certain of these embodiments, the randomized antigen polypeptide is N-terminal to the β2-microglobulin polypeptide on the single polypeptide. In certain of these embodiments, the randomized antigen polypeptide is C-terminal to the β2-microglobulin polypeptide on the single polypeptide.

In some embodiments, each of the antigen polypeptide-Beta-2 (β2) microglobulin polypeptide complexes of the plurality of the antigen polypeptide-Beta-2 (β2) microglobulin polypeptide complexes do not comprise an epitope tag. In some embodiments, at least one of the antigen polypeptide-Beta-2 (β2) microglobulin polypeptide complexes of the plurality of antigen polypeptide-Beta-2 (β2) microglobulin polypeptide complexes comprise an epitope tag. In some embodiments, at least one of the HLA-antigen complexes of the plurality of HLA-antigen complexes does not comprise an epitope tag and at least one of the HLA-antigen complexes of the plurality of HLA-antigen complexes does comprise an epitope tag. In some embodiments, the epitope tag comprises a FLAG tag, a c-MYC tag, a HIS-tag, a hemagglutinin (HA) tag, a VSVg tag, or a V5 tag.

In some embodiments, the antigen polypeptide-Beta-2 (β2) microglobulin polypeptide complexes each comprise a membrane tethering domain. In some embodiments, the membrane tethering domain comprises Aga2. In some embodiments, the antigen screening library is expressed on a plurality of cells.

In some embodiments, the plurality of cells are a plurality of yeast cells. In some embodiments, the plurality of yeast cells are a plurality of yeast cells of the EBY100 strain of Saccharomyces cerevisiae.

In some embodiments, each cell of the plurality of cells expresses a specific antigen polypeptide-Beta-2 (β2) microglobulin polypeptide complex.

Provided herein in some embodiments are a plurality of nucleic acids encoding the antigen screening libraries in accordance with the present technology.

In some embodiments, the HLA polypeptide of the HLA-antigen complex is encoded by a nucleic acid that is at least about 85%, 87.5%, 90%, 95%, 97%, 98%, or 99% homologous to any one of SEQ ID NOs: 456 to 484. In some embodiments, the randomized antigen polypeptide of the HLA-antigen complex is encoded by a nucleic acid set forth in any one of SEQ ID NOs: 210 to 426.

In some embodiments, the plurality of nucleic acids is expressed by a plurality of cells.

Provided herein in some embodiments are a plurality of cells expressing the antigen screening library in accordance with the present technology.

In some embodiments, the plurality of cells is a plurality of yeast cells. In some embodiments, the plurality of yeast cells is a plurality of cells of the EBY100 strain of Saccharomyces cerevisiae. In some embodiments, each cell of the plurality of cells comprises a nucleic acid of the plurality of nucleic acids encoding a specific of HLA-antigen complex.

Provided herein in some embodiments are methods of selecting an antigen comprising contacting the plurality of cells in accordance with the present technology with a T cell receptor (TCR).

In some embodiments, the TCR is immobilized on a substrate. In some embodiments, the TCR is expressed by a cell.

In some embodiments, the selection is repeated for 2, 3, 4, or 5 cycles.

In some embodiments, the antigen is a polypeptide antigen. In some embodiments, the antigen is a polypeptide antigen that does not naturally occur. In some embodiments, the antigen is a polypeptide antigen that does not naturally occur in a human.

### BRIEF DESCRIPTION OF THE DRAWINGS

FIG. 1A illustrates a schematic of an HLA antigen polypeptide construct coupled to a yeast cell in accordance with some embodiments of the present technology.

FIG. 1B illustrates an exemplary, non-limiting, embodiment of an HLA antigen polypeptide construct tethered to a cell in accordance with some embodiments of the present technology.

FIG. 2 illustrates an exemplary, non-limiting, depiction of a process for selecting a specific randomized antigen polypeptide that interacts with a specific T cell receptor in accordance with some embodiments of the present technology.

FIGS. 3A and 3B are maps of an example pCT vector (FIG. 3A) and an example pYAL vector (FIG. 3B).

FIG. 4 illustrates characterization by flow cytometry of peptide-HLA (pHLA) expression on yeast surface for a plurality of allotypes in accordance with some embodiments of the present technology.

### DETAILED DESCRIPTION

Described herein are antigen screening libraries useful for selection and/or identification of polypeptide ligands for T cell receptors (TCRs). In many cases, the antigen screening libraries are useful to discover polypeptide antigens that are capable of interacting with and stimulating human T cells as TCR ligands, including both endogenous TCR antigens and non-endogenous TCR antigens which may be novel TCR antigens and/or novel epitopes. Such novel antigens and/or novel epitopes are useful, at least for example, to stimulate one or more TCRs on T cells that may have become exhausted or anergized, and revive immune responses against cancer, tumors, or chronic viral infections. Accordingly, the present disclosure includes peptide library display, such as randomized peptide antigen libraries, in the context of a given HLA to determine the specificities and general recognition properties of TCRs restricted to HLA-mediated peptide recognition.

Once expressed using the methodologies described herein, a randomized peptide antigen library may be displayed by HLA molecules that are expressed on the surface of cells. In general, the cells that display these HLA-antigen polypeptide complexes are not normal antigen presenting cells of a host's immune system but rather are cells that can easily be transformed, transfected, transduced, and/or electroporated with a nucleic acid encoding an HLA-antigen polypeptide, including without limitation, insect cells, yeast cells, and bacterial cells. In some embodiments, the randomized peptide antigen library is expressed by yeast cells. A mixture of plasmids that encode at least 10⁴, 10⁵, 10⁶, 10⁷, 10⁸, 10⁹, 10¹⁰, 10¹¹, 10¹², 10¹³, 10¹⁴, or 10¹⁵ distinct polypeptide antigens, and either one or a plurality of different HLA molecules, are transformed into yeast cells. Following transformation with the randomized peptide antigen library, the yeast cells that express the HLA-antigen polypeptide complex library are then contacted by a TCR, or other macromolecule having one or more antigen binding domains, serving as a bait. The TCRs are either (1) expressed by a cell or (2) recombinantly produced and, optionally, multimerized and/or immobilized, on a solid structure, such as a bead, or via a protein scaffold such as streptavidin or streptavidin conjugated dextran (referenced as the selection reagent). The cells expressing HLA-antigen polypeptide complexes that interact with the TCR selection reagent can be selected by an appropriate modality, and after 2, 3, 4, 5, 6, 7 or more rounds of enrichment (e.g., cycles) the nucleic acids encoding the HLA-antigen polypeptide complexes can be extracted from the enriched cells and sequencing can be performed to determine the polypeptide antigens that have been enriched. The enriched polypeptide antigens define the structural attributes that interact with a given TCR.

In some embodiments, the present disclosure includes an antigen screening library which comprises a plurality of HLA-antigen polypeptide complexes. In some embodiments, the HLA-antigen polypeptide complexes comprise (a) an HLA polypeptide, the HLA polypeptide comprising a peptide binding cleft; (b) a randomized antigen polypeptide comprising an amino acid sequence set forth in any one of SEQ ID NOs: 1 to 194, wherein the randomized antigen polypeptide is selected to specifically bind to the peptide binding cleft of the HLA polypeptide; and (c) a beta-2 (β2) microglobulin polypeptide. Also provided herein are derivatives of randomized peptide antigens and libraries thereof, compositions thereof, pharmaceutical compositions thereof, and uses of the same. Also provided herein are nucleic acid sequences encoding one or more randomized peptide antigen libraries disclosed herein and derivatives thereof, and methods for expressing the one or more randomized peptide antigen libraries, peptides thereof, and derivatives thereof in one or more cells.

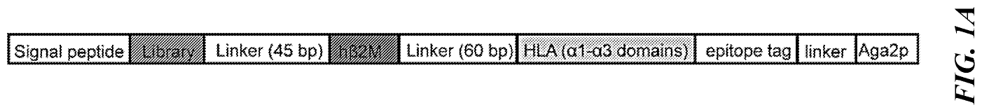

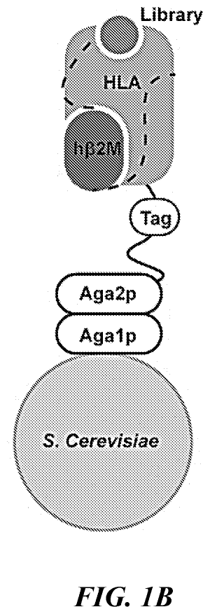

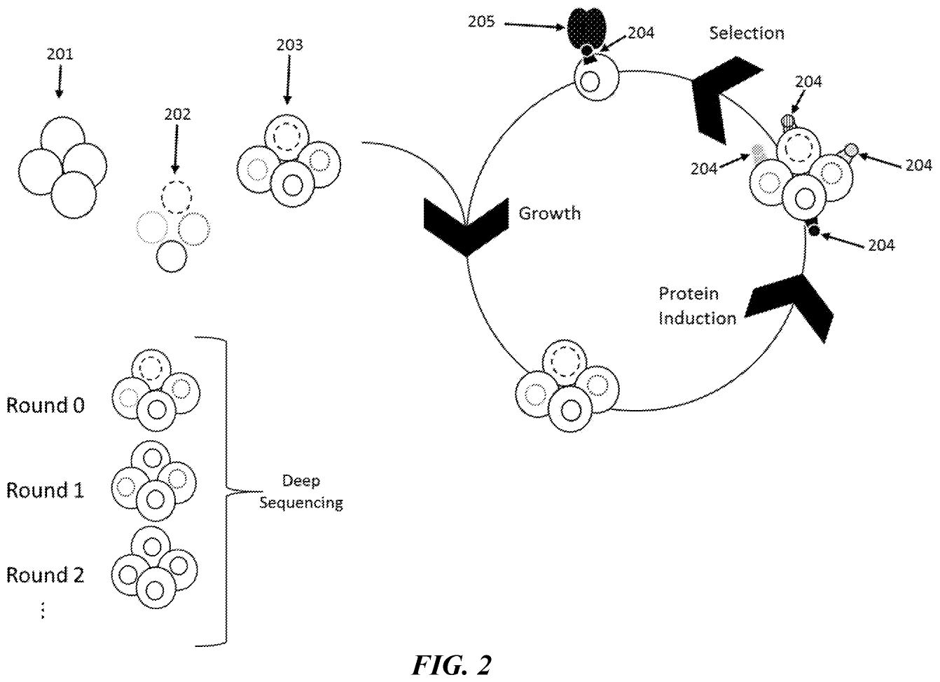

As set forth in the examples provided herein, a randomized peptide antigen library was designed (Example 1) and includes nucleic acid constructs (FIG. 1A) and peptide constructs tethered to a cell, such as a yeast cell (FIG. 1B). Expression of pHLA was characterized and validated using a yeast display (YD) system (Example 2). These pHLAs can interact with a TCR and determining whether interaction occurs can be determined with one or more processes described herein, such as was performed using the process illustrated in FIG. 2. Expression of pHLA was validated by flow cytometry (Example 2, Level 1) and can further be functionally validated by screening the randomized peptide antigen library using a candidate allotype-matched TCR (Example 2, Level 2).

The following description of the invention is merely intended to illustrate various embodiments of the present disclosure. As such, the specific modifications discussed are not to be construed as limitations on the scope of the present disclosure. It will be apparent to one skilled in the art that various equivalents, changes, and modifications may be made without departing from the scope of the present disclosure, and it is understood that such equivalent embodiments are to be included herein.

All references listed herein are incorporated by reference, in their entirety. Methods and apparatuses are provided here by way of example and are not intended to be limiting to the present disclosure.

### Certain Definitions

In the following description, some specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The terms “peptide,” “polypeptide,” and “protein” are used interchangeably to refer to a polymer of amino acid residues, and are not limited to a minimum length, though a number of amino acid residues may be specified (e.g., 9mer is nine amino acid residues). Polypeptides may include amino acid residues including natural and/or non-natural amino acid residues. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L and norleucine (Nle).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, the terms “homologous,” “homology,” or “percent homology” when used herein to describe to a nucleic acid sequence, relative to a reference sequence, can be determined using the formula described by Karlin & Altschul 1990, modified as in Karlin & Altschul 1993. Such a formula is incorporated into the basic local alignment search tool (BLAST) programs of Altschul 1990. Percent homology of sequences can be determined using the most recent version of BLAST, as of the filing date of this application.

“T cell receptor” (TCR), refers to an antigen/MHC binding heterodimeric protein product of a vertebrate, e.g. mammalian, TCR gene complex, including the human TCR α, β, γ and δ chains. For example, the complete sequence of the human (3 TCR locus has been sequenced, as published by Rowen 1996; the human TCR locus has been sequenced and resequenced, for example see Mackelprang 2006; see a general analysis of the T-cell receptor variable gene segment families in Arden 1995; each of which is herein specifically incorporated by reference for the sequence information provided and referenced in the publication.

“Bait” refers to a TCR or “other macromolecule having one or more antigen binding domains” that binds to an antigen of the present technology. The other macromolecule having one or more antigen binding domains is an antibody, a DARPin, or a synthetic molecule, including aptamers. The antigen binding domain binds a peptide, such as one or more of the HLA-peptide complexes of the present technology, or a nucleic acid, such as DNA and RNA.

“Exogenous” with respect to a nucleic acid or polynucleotide indicates that the nucleic acid is part of a recombinant nucleic acid construct or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., nonnative regulatory sequences flanking a native sequence in a recombinant nucleic acid construct. In addition, stably transformed exogenous nucleic acids typically are integrated at positions other than the position where the native sequence is found. The exogenous elements may be added to a construct, for example, using genetic recombination. Genetic recombination is the breaking and rejoining of DNA strands to form new molecules of DNA encoding a novel set of genetic information.

As used herein the term “about” refers to an amount that is near the stated amount by 10%.

### Structural Characteristics of the HLA-Antigen Polypeptide Complexes

Disclosed herein are antigen screening libraries, such as randomized peptide antigen libraries, which include a plurality of HLA-antigen polypeptide complexes. The HLA-antigen polypeptide complexes of the current disclosure minimally comprise at least three constituents: (a) a randomized antigen polypeptide, (b) a major histocompatibility class I (MHC I) HLA molecule, and (c) a β2-microglobulin. In some embodiments, the randomized antigen polypeptide of (a) is randomized having at least one or more residues conserved that serve as anchor residues to bind to an HLA molecule of a specific type. Exemplary, but not limiting, randomized antigen polypeptide antigens and the HLA type with which they associate are shown in Table 1 and given by SEQ ID NOs: 1 to 194 and Table 2 and given by SEQ ID NOs: 195 to 209. In some embodiments, the randomized polypeptide antigens comprises a sequence that is at least about 70%, 75%, 80%, 85%, 87%, 87.5%, 90%, 95%, 97%, 97.5%, 98%, 98.5%, 99%, 99.5%, or 100% identical to any one of, but not limited to, the amino acid sequences set forth in any one of SEQ ID NOs: 1 to 194 and SEQ ID NOs: 195 to 209. In some embodiments, the randomized polypeptide antigens comprise a sequence identical to any one of those set forth in any one of SEQ ID NOs: 1 to 194 and SEQ ID NOs: 195 to 209. Also envisioned within the present disclosure are randomized polypeptide antigen truncations that have 1, 2, 3, 4, 5, 6, 7, 8, 9, 10, 15, 20, or 25 amino acids truncated from the N-terminus or truncated from the C-terminus of any one of SEQ ID NOs: 1 to 194 and SEQ ID NOs: 195 to 209. In some embodiments, the HLA molecule of (b) is a HLA polypeptide and comprises a peptide binding cleft. Once expressed, in some embodiments, the randomized antigen polypeptide of (a) binds the HLA polypeptide of (b) at the peptide binding cleft.

TABLE 1
Polypeptide Antigen Sequences Sorted By HLA Type

| HLA type | SEQ ID NO: | Sequence |
|---|---|---|
| HLA A3 | 1 | X(V/L/M)XXXXXK |
| | 2 | X(V/L/M)XXXXXXK |
| | 3 | X(V/L/M)XXXXXXXK |
| | 4 | X(V/L/M)XXXXXXXXK |
| | 5 | X(V/L/M)XXXXXXXXXK |
| HLA A11 | 6 | X(V/L/F)XXXXX(K/R) |
| | 7 | X(V/L/F)XXXXXX(K/R) |
| | 8 | X(V/L/F)XXXXXXX(K/R) |
| | 9 | X(V/L/F)XXXXXXXX(K/R) |
| | 10 | X(V/L/F)XXXXXXXXX(K/R) |
| HLA A23 | 11 | XXXXXXXY |
| | 12 | XXXXXXXXY |
| | 13 | XXXXXXXXXY |
| | 14 | XXXXXXXXXXY |
| | 15 | XXXXXXXXXXXY |
| HLA A24 | 16 | X(F/Y)XXXXX(I/L/F) |
| | 17 | X(F/Y)XXXXXX(I/L/F) |
| | 18 | X(F/Y)XXXXXXX(I/L/F) |
| | 19 | X(F/Y)XXXXXXXX(I/L/F) |
| | 20 | X(F/Y)XXXXXXXXX(I/L/F) |
| HLA A26 | 21 | X(V/L/F)XXXXX(F/Y) |
| | 22 | X(I/T)XXXXX(F/Y) |
| | 23 | X(V/L/F)XXXXXX(F/Y) |
| | 24 | X(I/T)XXXXXX(F/Y) |
| | 25 | X(V/L/F)XXXXXXX(F/Y) |
| | 26 | X(I/T)XXXXXXX(F/Y) |
| | 27 | X(V/L/F)XXXXXXXX(F/Y) |
| | 28 | X(I/T)XXXXXXXX(F/Y) |
| | 29 | X(V/L/F)XXXXXXXXX(F/Y) |
| | 30 | X(I/T)XXXXXXXXX(F/Y) |
| HLA A30 | 31 | X(F/Y)XXXXX(L) |
| | 32 | X(F/Y)XXXXXX(L) |
| | 33 | X(F/Y)XXXXXXX(L) |
| | 34 | X(F/Y)XXXXXXXX(L) |
| | 35 | X(F/Y)XXXXXXXXX(L) |
| HLA A31 | 36 | XXXXXXX(K/R) |
| | 37 | XXXXXXXX(K/R) |
| | 38 | XXXXXXXXX(K/R) |
| | 39 | XXXXXXXXXX(K/R) |
| | 40 | XXXXXXXXXXX(K/R) |
| HLA A33 | 41 | XXXXXXX(K/R) |
| | 42 | XXXXXXXX(K/R) |
| | 43 | XXXXXXXXX(K/R) |
| | 44 | XXXXXXXXXX(K/R) |
| | 45 | XXXXXXXXXXX(K/R) |
| HLA A68 | 46 | X(V)XXXXX(K/R) |
| | 47 | X(V)XXXXXX(K/R) |
| | 48 | X(V)XXXXXXX(K/R) |
| | 49 | X(V)XXXXXXXX(K/R) |
| | 50 | X(V)XXXXXXXXX(K/R) |
| | 51 | X(T)XXXXX(K/R) |
| | 52 | X(T)XXXXXX(K/R) |
| | 53 | X(T)XXXXXXX(K/R) |
| | 54 | X(T)XXXXXXXX(K/R) |
| | 55 | X(T)XXXXXXXXX(K/R) |
| HLA B7 | 56 | XPXXXXXL |
| | 57 | XPXXXXXXL |
| | 58 | XPXXXXXXXL |
| | 59 | XPXXXXXXXXL |
| | 60 | XPXXXXXXXXXL |
| HLA B8 | 61 | XX(K)X(K/R)XX(L) |
| | 62 | XX(K)X(K/R)XXX(L) |
| | 63 | XX(K)X(K/R)XXXX(L) |
| | 64 | XX(K)X(K/R)XXXXX(L) |
| | 65 | XX(K)X(K/R)XXXXXX(L) |
| HLA B15 | 66 | X(Q/L)XXXXX(F/Y) |
| | 67 | X(Q/L)XXXXXX(F/Y) |
| | 68 | X(Q/L)XXXXXXX(F/Y) |
| | 69 | X(Q/L)XXXXXXXX(F/Y) |
| | 70 | X(Q/L)XXXXXXXXX(F/Y) |
| HLA B27 | 71 | X(R)XXXXX(F/Y) |
| | 72 | X(R)XXXXXX(F/Y) |
| | 73 | X(R)XXXXXXX(F/Y) |
| | 74 | X(R)XXXXXXXX(F/Y) |
| | 75 | X(R)XXXXXXXXX(F/Y) |
| HLA B35 | 76 | X(P)XXXXX(F/Y) |
| | 77 | X(P)XXXXXX(F/Y) |
| | 78 | X(P)XXXXXXX(F/Y) |
| | 79 | X(P)XXXXXXXX(F/Y) |
| | 80 | X(P)XXXXXXXXX(F/Y) |
| | 81 | X(P)XXXXX(M/L/I) |
| | 82 | X(P)XXXXXX(M/L/I) |
| | 83 | X(P)XXXXXXX(M/L/I) |
| | 84 | X(P)XXXXXXXX(M/L/I) |
| | 85 | X(P)XXXXXXXXX(M/L/I) |
| HLA B40 | 86 | X(E)XXXXX(L) |
| | 87 | X(E)XXXXXX(L) |
| | 88 | X(E)XXXXXXX(L) |
| | 89 | X(E)XXXXXXXX(L) |
| | 90 | X(E)XXXXXXXXX(L) |
| HLA B51 | 91 | X(A/G)XXXXX(V/I) |
| | 92 | X(A/G)XXXXXX(V/I) |
| | 93 | X(A/G)XXXXXXX(V/I) |
| | 94 | X(A/G)XXXXXXXX(V/I) |
| | 95 | X(A/G)XXXXXXXXX(V/I) |
| | 96 | X(P)XXXXX(V/I) |
| | 97 | X(P)XXXXXX(V/I) |
| | 98 | X(P)XXXXXXX(V/I) |
| | 99 | X(P)XXXXXXXX(V/I) |
| | 100 | X(P)XXXXXXXXX(V/I) |
| HLA B44 | 101 | X(E)XXXXX(F/Y) |
| | 102 | X(E)XXXXXX(F/Y) |
| | 103 | X(E)XXXXXXX(F/Y) |
| | 104 | X(E)XXXXXXXX(F/Y) |
| | 105 | X(E)XXXXXXXXX(F/Y) |
| HLA B53 | 106 | X(P)XXXXX(W) |
| | 107 | X(P)XXXXXX(W) |
| | 108 | X(P)XXXXXXX(W) |
| | 109 | X(P)XXXXXXXX(W) |
| | 110 | X(P)XXXXXXXXX(W) |
| | 111 | X(P)XXXXX(F/L) |
| | 112 | X(P)XXXXXX(F/L) |
| | 113 | X(P)XXXXXXX(F/L) |
| | 114 | X(P)XXXXXXXX(F/L) |
| | 115 | X(P)XXXXXXXXX(F/L) |
| HLA B57 | 116 | X(A/T/S)XXXXX(F/Y) |
| | 117 | X(A/T/S)XXXXXX(F/Y) |
| | 118 | X(A/T/S)XXXXXXX(F/Y) |
| | 119 | X(A/T/S)XXXXXXXX(F/Y) |
| | 120 | X(A/T/S)XXXXXXXXX(F/Y) |
| | 121 | X(A/T/S)XXXXX(W) |
| | 122 | X(A/T/S)XXXXXX(W) |
| | 123 | X(A/T/S)XXXXXXX(W) |
| | 124 | X(A/T/S)XXXXXXXX(W) |
| | 125 | X(A/T/S)XXXXXXXXX(W) |
| HLA C1 | 126 | X(L)XXXXX(L) |
| | 127 | X(L)XXXXXX(L) |
| | 128 | X(L)XXXXXXX(L) |
| | 129 | X(L)XXXXXXXX(L) |
| | 130 | X(L)XXXXXXXXX(L) |
| | 131 | X(A)XXXXXXX(L) |
| | 132 | X(A)XXXXXX(L) |
| | 133 | X(A)XXXXXXX(L) |
| | 134 | X(A)XXXXXXXX(L) |
| | 135 | X(A)XXXXXXXXX(L) |
| HLA C2 | 136 | X(A)XXXXX(L/V) |
| | 137 | X(A)XXXXXX(L/V) |
| | 138 | X(A)XXXXXXX(LN) |
| | 139 | X(A)XXXXXXXX(L/V) |
| | 140 | X(A)XXXXXXXXX(L/V) |
| | 141 | X(A)XXXXX(F/Y) |
| | 142 | X(A)XXXXXX(F/Y) |
| | 143 | X(A)XXXXXXX(F/Y) |
| | 144 | X(A)XXXXXXXX(F/Y) |
| | 145 | X(A)XXXXXXXXX(F/Y) |
| HLA C3 | 146 | XXXXXXX(L/F/M/I) |
| | 147 | XXXXXXXX(L/F/M/I) |
| | 148 | XXXXXXXXX(L/F/M/I) |
| | 149 | XXXXXXXXXX(L/F/M/I) |
| | 150 | XXXXXXXXXXX(L/F/M/I) |
| HLA C4 | 151 | X(Y/F)XXXXX(L/F/M/I) |
| | 152 | X(Y/F)XXXXXX(L/F/M/I) |
| | 153 | X(Y/F)XXXXXXX(L/F/M/I) |
| | 154 | X(Y/F)XXXXXXXX(L/F/M/I) |
| | 155 | X(Y/F)XXXXXXXXX(L/F/M/I) |
| | 156 | X(P)XXXXX(L/F/M/I) |
| | 157 | X(P)XXXXXX(L/F/M/I) |
| | 158 | X(P)XXXXXXX(L/F/M/I) |
| | 159 | X(P)XXXXXXXX(L/F/M/I) |
| | 160 | X(P)XXXXXXXXX(L/F/M/I) |
| HLA C5 | 161 | XX(D)XXXX(L/V) |
| | 162 | XX(D)XXXXX(L/V) |
| | 163 | XX(D)XXXXXX(LN) |
| | 164 | XX(D)XXXXXXX(L/V) |
| | 165 | XX(D)XXXXXXXX(L/V) |
| HLA C6 | 166 | XXXXXXX(L/V/I) |
| | 167 | XXXXXXXX(L/V/I) |
| | 168 | XXXXXXXXX(L/V/I) |
| | 169 | XXXXXXXXXX(L/V/I) |
| | 170 | XXXXXXXXXXX(L/V/I) |
| | 171 | XXXXXXX(Y) |
| | 172 | XXXXXXXX(Y) |
| | 173 | XXXXXXXXX(Y) |
| | 174 | XXXXXXXXXX(Y) |
| | 175 | XXXXXXXXXXX(Y) |
| HLA C7 | 176 | XXXXXXX(F/L) |
| | 177 | XXXXXXXX(F/L) |
| | 178 | XXXXXXXXX(F/L) |
| | 179 | XXXXXXXXXX(F/L) |
| | 180 | XXXXXXXXXXX(F/L) |
| | 181 | XXXXXXX(Y) |
| | 182 | XXXXXXXX(Y) |
| | 183 | XXXXXXXXX(Y) |
| | 184 | XXXXXXXXXX(Y) |
| | 185 | XXXXXXXXXXX(Y) |
| HLA C8 | 186 | XX(D)XXX(L) |
| | 187 | XX(D)XXXX(L) |
| | 188 | XX(D)XXXXX(L) |
| | 189 | XX(D)XXXXXX(L) |
| | 190 | XX(D)XXXXXXX(L) |
| HLA E | 191 | X(L/M)XXXXX(L/V) |
| | 192 | X(L/M)XXXXXX(LN) |
| | 193 | X(L/M)XXXXXXX(LN) |
| | 194 | X(L/M)XXXXXXXX(L/V) |

TABLE 2
Additional Polypeptide Antigen
Sequences for HLA A11

| SEQ ID NO: | Sequence |
|---|---|
| 195 | X(I/L/V)XXXXX(K/R) |
| 196 | X(I/L/V)XXXXXX(K/R) |
| 197 | X(I/L/V)XXXXXXX(K/R) |
| 198 | X(I/L/V)XXXXXXXX(K/R) |
| 199 | X(I/L/V)XXXXXXXXX(K/R) |
| 200 | X(Y/F)XXXXX(K/R) |
| 201 | X(Y/F)XXXXXX(K/R) |
| 202 | X(Y/F)XXXXXXX(K/R) |
| 203 | X(Y/F)XXXXXXXX(K/R) |
| 204 | X(Y/F)XXXXXXXXX(K/R) |
| 205 | X(N/Y)XXXXX(K/R) |
| 206 | X(N/Y)XXXXXX(K/R) |
| 207 | X(N/Y)XXXXXXX(K/R) |
| 208 | X(N/Y)XXXXXXXX(K/R) |
| 209 | X(N/Y)XXXXXXXXX(K/R) |

In some embodiments, antigen screening libraries of the present disclosure include (b) randomized antigen polypeptides encoded at least by, but not limited to, nucleotide sequences SEQ ID NOs: 210 to 411 provided at least in Table 4. In some embodiments, antigen screening libraries of the present disclosure include (b) randomized antigen polypeptides encoded at least by, but not limited to, nucleotide sequences SEQ ID NOs: 412 to 426 provided at least in Table 5. Nucleic acids that encode the randomized antigen polypeptides of (b) are encoded by a degenerate base sequence, effectively allowing any amino acid to be encoded at a given position corresponding to the degenerate base sequence. Each randomized antigen polypeptide has at least one conserved anchor position that is encoded by a restricted degenerate code, or a specific sequence, which allows the randomized antigen polypeptide to more efficiently interact with a certain HLA type. Having at least one conserved anchor position per randomized antigen polypeptide increases efficiency of formation of a randomized antigen polypeptide and HLA complex compared to formation of an HLA complex with a fully randomized antigen polypeptide. In some embodiments, 1, 2, or 3 of the amino acid residues of a randomized antigen polypeptide are constant. In some embodiments, the randomized antigen polypeptide antigens comprises a sequence that is at least about 70%, 75%, 80%, 85%, 87%, 87.5%, 90%, 95%, 97%, 97.5%, 98%, 98.5%, 99%, 99.5%, or 100% identical to any one of, but not limited to, the amino acid sequences set forth in any one of SEQ ID NOs: 210 to 411 and SEQ ID NOs: 412 to 426. In some embodiments, the randomized antigen polypeptide antigens comprise a sequence identical to any one of those set forth in any one of SEQ ID NOs: 210 to 411 and SEQ ID NOs: 412 to 426. Also envisioned within the present disclosure are randomized antigen polypeptide antigen polypeptide truncations that have 1, 2, 3, 4, 5, 6, 7, 8, 9, 10, 15, 20, or 25 amino acids truncated from the N-terminus or truncated from the C-terminus of any one of SEQ ID NOs: 210 to 411 and SEQ ID NOs: 412 to 426.

In some embodiments, amino acid residues of a randomized antigen polypeptide vary by 2, 3, or 4 different amino acids. For example, referring to Table 1, the second and the last position of a randomized antigen polypeptide that binds to HLA-A2 will comprise leucine or methionine; and leucine, methionine, or valine, respectively.

The amino acid sequences in Tables 1 and 2 above include random amino acid residues (‘X’) and explicitly defined amino acids located at residues referred to collectively as anchor positions. The anchor positions specified in the library design can be altered, for example, based on amino acid substitutions set forth in Table 3. One of ordinary skill in the art would appreciate that possible substitutions for X residue in the amino acid sequences of Tables 1 and 2 are not limited and can include additional substitutions without departing from the scope of the disclosure. For example, amino acid substitutions can be used to identify important residues of the peptide sequence that contribute to binding of the HLA or to constrain of expand the members of the library described herein.

Conservative modifications will produce peptides having functional and chemical characteristics similar to those of the peptide from which such modifications are made. In contrast, substantial modifications in the functional and/or chemical characteristics of the peptides may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the size of the molecule.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan 1998 and Sasaki & Sutoh 1998, which discuss alanine scanning mutagenesis).

Desired amino acid substitutions (whether conservative or non-conservative) can be determined by those skilled in the art at the time such substitutions are desired. Exemplary amino acid substitutions are set forth in Table 3.

TABLE 3
Amino Acid Substitutions

| Original | |
|---|---|
| Residues | Exemplary Substitutions |
| Ala (A) | Val, Leu, Ile |
| Arg (R) | Lys, Gln, Asn |
| Asn (N) | Gln |
| Asp (D) | Glu |
| Cys (C) | Ser, Ala |
| Gln (Q) | Asn |
| Glu (E) | Asp |
| Gly (G) | Pro, Ala |
| His (H) | Asn, Gln, Lys, Arg |
| Ile (I) | Leu, Val, Met, Ala, Phe, Norleucine (Nle) |
| Leu (L) | Norleucine (Nle), Ile, Val, Met, Ala, Phe |
| Lys (K) | Arg, 1,4 Diaminobutyric Acid (Dab), Gln, Asn |
| Met (M) | Leu, Phe, Ile |
| Phe (F) | Leu, Val, Ile, Ala, Tyr |
| Pro (P) | Ala |
| Ser (S) | Thr, Ala, Cys |
| Thr (T) | Ser |
| Trp (W) | Tyr, Phe |
| Tyr (Y) | Trp, Phe, Thr, Ser |
| Val (V) | Ile, Met, Leu, Phe, Ala, Norleucine (Nle) |

In certain embodiments, conservative amino acid substitutions also encompass non-naturally occurring amino acid residues which are typically incorporated by chemical peptide synthesis rather than by synthesis in biological systems.

As noted in the foregoing section “Certain Definitions,” naturally occurring residues may be divided into classes based on common sidechain properties that may be useful for modifications of sequence. For example, non-conservative substitutions may involve the exchange of a member of one of these classes for a member from another class. Such substituted residues may be introduced into regions of the peptide that are homologous with non-human orthologs, or into the non-homologous regions of the molecule. In addition, one may also make modifications using P or G for the purpose of influencing chain orientation.

In making such modifications, the hydropathic index of amino acids may be considered. Each amino acid has been assigned a hydropathic index on the basis of their hydrophobicity and charge characteristics; these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (Kyte & Doolittle 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. The greatest local average hydrophilicity of a protein, as governed by the hydrophilicity of its adjacent amino acids, correlates with its immunogenicity and antigenicity, i.e. with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. One may also identify epitopes from primary amino acid sequences on the basis of hydrophilicity. These regions are also referred to as “epitopic core regions.”

A skilled artisan will be able to determine suitable variants of the polypeptide as set forth in the foregoing sequences using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of a peptide to similar peptides. With such a comparison, one can identify residues and portions of the molecules that are conserved among similar polypeptides. It will be appreciated that changes in areas of a peptide that are not conserved relative to such similar peptides would be less likely to adversely affect the biological activity and/or structure of the peptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the peptide structure.

Additionally, one skilled in the art can review structure-function studies identifying residues in similar peptides that are important for activity or structure. In view of such a comparison, one can predict the importance of amino acid residues in a peptide that correspond to amino acid residues that are important for activity or structure in similar peptides. One skilled in the art may opt for chemically similar amino acid substitutions for such predicted important amino acid residues of the peptides.

One skilled in the art can also analyze the three-dimensional structure and amino acid sequence in relation to that structure in similar polypeptides. In view of that information, one skilled in the art may predict the alignment of amino acid residues of a peptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such data could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided, either alone or in combination with other mutations.

A number of scientific publications have been devoted to the prediction of secondary structure (see, e.g., Moult 1996; Chou & Fasman 1974a; Chou & Fasman 1974b; Chou & Fasman 1978a; Chou & Fasman 1978b; and Chou & Fasman 1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure (Holm & Sander 1999). It has been suggested that there are a limited number of folds in a given polypeptide or protein and that once a critical number of structures have been resolved, structural prediction will gain dramatically in accuracy (Brenner 1997).

Additional methods of predicting secondary structure include “threading” (Jones 1997; Sippl & Flockner 1996), “profile analysis” (Bowie 1991; Gribskov 1987; Gribskov 1990), and “evolutionary linkage” (Holm & Sander 1999; Brenner 1997).

TABLE 4
Nucleic Acid Sequences Encoding
Randomized Polypeptide Antigens

| HLA | SEQ | |
|---|---|---|
| type | ID NO: | Sequence |
| HLA A2 | 210 | nnkmtgnnknnknnknnknnkntg |
| | 211 | nnkmtgnnknnknnknnknnknnkntg |
| | 212 | nnkmtgnnknnknnknnknnknnknnkntg |
| | 213 | nnkmtgnnknnknnknnknnknnknnknnkntg |
| HLA A1 | 214 | nnknnkgaknnknnknnknnknnktat |
| | 215 | nnknnkgaknnknnknnknnknnknnktat |
| | 216 | nnknnkgaknnknnknnknnknnknnknnktat |
| HLA A3 | 217 | nnkvtgnnknnknnknnknnkaaa |
| | 218 | nnkvtgnnknnknnknnknnknnkaaa |
| | 219 | nnkvtgnnknnknnknnknnknnknnkaaa |
| | 220 | nnkvtgnnknnknnknnknnknnknnknnkaaa |
| | 221 | nnkvtgnnknnknnknnknnknnknnknnknnkaaa |
| HLA A11 | 222 | nnkbttnnknnknnknnknnkara |
| | 223 | nnkbttnnknnknnknnknnknnkara |
| | 224 | nnkbttnnknnknnknnknnknnknnkara |
| | 225 | nnkbttnnknnknnknnknnknnknnknnkara |
| | 226 | nnkbttnnknnknnknnknnknnknnknnknnkara |
| HLA A23 | 227 | nnknnknnknnknnknnknnktay |
| | 228 | nnknnknnknnknnknnknnknnktay |
| | 229 | nnknnknnknnknnknnknnknnknnktay |
| | 230 | nnknnknnknnknnknnknnknnknnknnktay |
| | 231 | nnknnknnknnknnknnknnknnknnknnknnktay |
| HLA A24 | 232 | nnktwtnnknnknnknnknnkhtt |
| | 233 | nnktwtnnknnknnknnknnknnkhtt |
| | 234 | nnktwtnnknnknnknnknnknnknnkhtt |
| | 235 | nnktwtnnknnknnknnknnknnknnknnkhtt |
| | 236 | nnktwtnnknnknnknnknnknnknnknnknnkhtt |
| HLA A26 | 237 | nnkbthnnknnknnknnknnktwy |
| | 238 | nnkayynnknnknnknnknnktwy |
| | 239 | nnkbthnnknnknnknnknnknnktwy |
| | 240 | nnkayynnknnknnknnknnknnktwy |
| | 241 | nnkbthnnknnknnknnknnknnknnktwy |
| | 242 | nnkayynnknnknnknnknnknnknnktwy |
| | 243 | nnkbthnnknnknnknnknnknnknnknnktwy |
| | 244 | nnkayynnknnknnknnknnknnknnknnktwy |
| | 245 | nnkbthnnknnknnknnknnknnknnknnknnktwy |
| | 246 | nnkayynnknnknnknnknnknnknnknnknnktwy |
| HLA A30 | 247 | nnktwynnknnknnknnknnkctn |
| | 248 | nnktwynnknnknnknnknnknnkctn |
| | 249 | nnktwynnknnknnknnknnknnknnkctn |
| | 250 | nnktwynnknnknnknnknnknnknnknnkctn |
| | 251 | nnktwynnknnknnknnknnknnknnknnknnkctn |
| HLA A31 | 252 | nnknnknnknnknnknnknnkarr |
| | 253 | nnknnknnknnknnknnknnknnkarr |
| | 254 | nnknnknnknnknnknnknnknnknnkarr |
| | 255 | nnknnknnknnknnknnknnknnknnknnkarr |
| | 256 | nnknnknnknnknnknnknnknnknnknnknnkarr |
| HLA A33 | 257 | nnknnknnknnknnknnknnkarr |
| | 258 | nnknnknnknnknnknnknnknnkarr |
| | 259 | nnknnknnknnknnknnknnknnknnkarr |
| | 260 | nnknnknnknnknnknnknnknnknnknnkarr |
| | 261 | nnknnknnknnknnknnknnknnknnknnknnkarr |
| HLA A68 | 262 | nnkgttnnknnknnknnknnkarr |
| | 263 | nnkgttnnknnknnknnknnknnkarr |
| | 264 | nnkgttnnknnknnknnknnknnknnkarr |
| | 265 | nnkgttnnknnknnknnknnknnknnknnkarr |
| | 266 | nnkgttnnknnknnknnknnknnknnknnknnkarr |
| | 267 | nnkactnnknnknnknnknnkarr |
| | 268 | nnkactnnknnknnknnknnknnkarr |
| | 269 | nnkactnnknnknnknnknnknnknnkarr |
| | 270 | nnkactnnknnknnknnknnknnknnknnkarr |
| | 271 | nnkactnnknnknnknnknnknnknnknnknnkarr |
| HLA B7 | 272 | nnkcctnnknnknnknnknnkctt |
| | 273 | nnkcctnnknnknnknnknnknnkctt |
| | 274 | nnkcctnnknnknnknnknnknnknnkctt |
| | 275 | nnkcctnnknnknnknnknnknnknnknnkctt |
| | 276 | nnkcctnnknnknnknnknnknnknnknnknnkctt |
| HLA B8 | 277 | nnknnkaaannkarannknnkctt |
| | 278 | nnknnkaaannkarannknnknnkctt |
| | 279 | nnknnkaaannkarannknnknnknnkctt |
| | 280 | nnknnkaaannkarannknnknnknnknnkctt |
| | 281 | nnknnkaaannkarannknnknnknnknnknnkctt |
| HLA B15 | 282 | nnkcwannknnknnknnknnktwt |
| | 283 | nnkcwannknnknnknnknnknnktwt |
| | 284 | nnkcwannknnknnknnknnknnknnktwt |
| | 285 | nnkcwannknnknnknnknnknnknnknnktwt |
| | 286 | nnkcwannknnknnknnknnknnknnknnknnktwt |
| HLA B27 | 287 | nnkagannknnknnknnknnktwt |
| | 288 | nnkagannknnknnknnknnknnktwt |
| | 289 | nnkagannknnknnknnknnknnknnktwt |
| | 290 | nnkagannknnknnknnknnknnknnknnktwt |
| | 291 | nnkagannknnknnknnknnknnknnknnknnktwt |
| HLA B35 | 292 | nnkcctnnknnknnknnknnktwt |
| | 293 | nnkcctnnknnknnknnknnknnktwt |
| | 294 | nnkcctnnknnknnknnknnknnknnktwt |
| | 295 | nnkcctnnknnknnknnknnknnknnknnktwt |
| | 296 | nnkcctnnknnknnknnknnknnknnknnknnktwt |
| | 297 | nnkcctnnknnknnknnknnkmtr |
| | 298 | nnkcctnnknnknnknnknnknnkmtr |
| | 299 | nnkcctnnknnknnknnknnknnknnkmtr |
| | 300 | nnkcctnnknnknnknnknnknnknnknnkmtr |
| | 301 | nnkcctnnknnknnknnknnknnknnknnknnkmtr |
| HLA B40 | 302 | nnkgaannknnknnknnknnkctt |
| | 303 | nnkgaannknnknnknnknnknnkctt |
| | 304 | nnkgaannknnknnknnknnknnknnkctt |
| | 305 | nnkgaannknnknnknnknnknnknnknnkctt |
| | 306 | nnkgaannknnknnknnknnknnknnknnknnkctt |
| HLA B51 | 307 | nnkgstnnknnknnknnknnkrtt |
| | 308 | nnkgstnnknnknnknnknnknnkrtt |
| | 309 | nnkgstnnknnknnknnknnknnknnkrtt |
| | 310 | nnkgstnnknnknnknnknnknnknnknnkrtt |
| | 311 | nnkgstnnknnknnknnknnknnknnknnknnkrtt |
| | 312 | nnkcctnnknnknnknnknnkrtt |
| | 313 | nnkcctnnknnknnknnknnknnkrtt |
| | 314 | nnkcctnnknnknnknnknnknnknnkrtt |
| | 315 | nnkcctnnknnknnknnknnknnknnknnkrtt |
| | 316 | nnkcctnnknnknnknnknnknnknnknnknnkrtt |
| HLA B44 | 317 | nnkgaannknnknnknnknnktwt |
| | 318 | nnkgaannknnknnknnknnknnktwt |
| | 319 | nnkgaannknnknnknnknnknnknnktwt |
| | 320 | nnkgaannknnknnknnknnknnknnknnktwt |
| | 321 | nnkgaannknnknnknnknnknnknnknnknnktwt |
| HLA B53 | 322 | nnkcctnnknnknnknnknnktgg |
| | 323 | nnkcctnnknnknnknnknnknnktgg |
| | 324 | nnkcctnnknnknnknnknnknnknnktgg |
| | 325 | nnkcctnnknnknnknnknnknnknnknnktgg |
| | 326 | nnkcctnnknnknnknnknnknnknnknnknnktgg |
| | 327 | nnkcctnnknnknnknnknnkytt |
| | 328 | nnkcctnnknnknnknnknnknnkytt |
| | 329 | nnkcctnnknnknnknnknnknnknnkytt |
| | 330 | nnkcctnnknnknnknnknnknnknnknnkytt |
| | 331 | nnkcctnnknnknnknnknnknnknnknnknnkytt |
| HLA B57 | 332 | nnkdctnnknnknnknnknnktwt |
| | 333 | nnkdctnnknnknnknnknnknnktwt |
| | 334 | nnkdctnnknnknnknnknnknnknnktwt |
| | 335 | nnkdctnnknnknnknnknnknnknnknnktwt |
| | 336 | nnkdctnnknnknnknnknnknnknnknnknnktwt |
| | 337 | nnkdctnnknnknnknnknnktgg |
| | 338 | nnkdctnnknnknnknnknnknnktgg |
| | 339 | nnkdctnnknnknnknnknnknnknnktgg |
| | 340 | nnkdctnnknnknnknnknnknnknnknnktgg |
| | 341 | nnkdctnnknnknnknnknnknnknnknnknnktgg |
| HLA C1 | 342 | nnkttannknnknnknnknnktta |
| | 343 | nnkttannknnknnknnknnknnktta |
| | 344 | nnkttannknnknnknnknnknnknnktta |
| | 345 | nnkttannknnknnknnknnknnknnknnktta |
| | 346 | nnkttannknnknnknnknnknnknnknnknnktta |
| | 347 | nnkgctnnknnknnknnknnktta |
| | 348 | nnkgctnnknnknnknnknnknnktta |
| | 349 | nnkgctnnknnknnknnknnknnknnktta |
| | 350 | nnkgctnnknnknnknnknnknnknnknnktta |
| | 351 | nnkgctnnknnknnknnknnknnknnknnknnktta |
| HLA C2 | 352 | nnkgctnnknnknnknnknnkstc |
| | 353 | nnkgctnnknnknnknnknnknnkstc |
| | 354 | nnkgctnnknnknnknnknnknnknnkstc |
| | 355 | nnkgctnnknnknnknnknnknnknnknnkstc |
| | 356 | nnkgctnnknnknnknnknnknnknnknnknnkstc |
| | 357 | nnkgctnnknnknnknnknnktwt |
| | 358 | nnkgctnnknnknnknnknnknnktwt |
| | 359 | nnkgctnnknnknnknnknnknnknnktwt |
| | 360 | nnkgctnnknnknnknnknnknnknnknnktwt |
| | 361 | nnkgctnnknnknnknnknnknnknnknnknnktwt |
| HLA C3 | 362 | nnknnknnknnknnknnknnkhtk |
| | 363 | nnknnknnknnknnknnknnknnkhtk |
| | 364 | nnknnknnknnknnknnknnknnknnkhtk |
| | 365 | nnknnknnknnknnknnknnknnknnknnkhtk |
| | 366 | nnknnknnknnknnknnknnknnknnknnknnkhtk |
| HLA C4 | 367 | nnktwtnnknnknnknnknnkhtk |
| | 368 | nnktwtnnknnknnknnknnknnkhtk |
| | 369 | nnktwtnnknnknnknnknnknnknnkhtk |
| | 370 | nnktwtnnknnknnknnknnknnknnknnkhtk |
| | 371 | nnktwtnnknnknnknnknnknnknnknnknnkhtk |
| | 372 | nnkcctnnknnknnknnknnkhtk |
| | 373 | nnkcctnnknnknnknnknnknnkhtk |
| | 374 | nnkcctnnknnknnknnknnknnknnkhtk |
| | 375 | nnkcctnnknnknnknnknnknnknnknnkhtk |
| | 376 | nnkcctnnknnknnknnknnknnknnknnknnkhtk |
| HLA C5 | 377 | nnknnkgatnnknnknnknnkstt |
| | 378 | nnknnkgatnnknnknnknnknnkstt |
| | 379 | nnknnknnkgatnnknnknnknnknnkstt |
| | 380 | nnknnknnknnkgatnnknnknnknnknnkstt |
| | 381 | nnknnknnknnkgatnnknnknnknnknnknnkstt |
| HLA C6 | 382 | nnknnknnknnknnknnknnkvtt |
| | 383 | nnknnknnknnknnknnknnknnkvtt |
| | 384 | nnknnknnknnknnknnknnknnknnkvtt |
| | 385 | nnknnknnknnknnknnknnknnknnknnkvtt |
| | 386 | nnknnknnknnknnknnknnknnknnknnknnkvtt |
| | 387 | nnknnknnknnknnknnknnktat |
| | 388 | nnknnknnknnknnknnknnknnktat |
| | 389 | nnknnknnknnknnknnknnknnknnktat |
| | 390 | nnknnknnknnknnknnknnknnknnknnktat |
| | 391 | nnknnknnknnknnknnknnknnknnknnknnktat |
| HLA C7 | 392 | nnknnknnknnknnknnknnkytt |
| | 393 | nnknnknnknnknnknnknnknnkytt |
| | 394 | nnknnknnknnknnknnknnknnknnkytt |
| | 395 | nnknnknnknnknnknnknnknnknnknnkytt |
| | 396 | nnknnknnknnknnknnknnknnknnknnknnkytt |
| | 397 | nnknnknnknnknnknnknnktat |
| | 398 | nnknnknnknnknnknnknnknnktat |
| | 399 | nnknnknnknnknnknnknnknnknnktat |
| | 400 | nnknnknnknnknnknnknnknnknnknnktat |
| | 401 | nnknnknnknnknnknnknnknnknnknnknnktat |
| HLA C8 | 402 | nnknnkgatnnknnknnkctt |
| | 403 | nnknnkgatnnknnknnknnkctt |
| | 404 | nnknnkgatnnknnknnknnknnkctt |
| | 405 | nnknnkgatnnknnknnknnknnknnkat |
| | 406 | nnknnkgatnnknnknnknnknnknnknnkat |
| HLA E | 407 | nnkmtgnnknnknnknnknnkstt |
| | 408 | nnkmtgnnknnknnknnknnknnkstt |
| | 409 | nnkmtgnnknnknnknnknnknnknnkstt |
| | 410 | nnkmtgnnknnknnknnknnknnknnknnkstt |
| | 411 | nnkmtgnnknnknnknnknnknnknnknnknnkstt |

TABLE 5
Additional Nucleic Acid Sequences Encoding
Randomized Polypeptide Antigens for HLA A11

| SEQ ID NO: | Sequence |
|---|---|
| 412 | nnkvttnnknnknnknnknnkara |
| 413 | nnkvttnnknnknnknnknnknnkara |
| 414 | nnkvttnnknnknnknnknnknnknnkara |
| 415 | nnkvttnnknnknnknnknnknnknnknnkara |
| 416 | nnkvttnnknnknnknnknnknnknnknnknnkara |
| 417 | nnktwtnnknnknnknnknnknnkara |
| 418 | nnktwtnnknnknnknnknnkara |
| 419 | nnktwtnnknnknnknnknnknnknnkara |
| 420 | nnktwtnnknnknnknnknnknnknnknnkara |
| 421 | nnktwtnnknnknnknnknnknnknnknnknnkara |
| 422 | nnkwatnnknnknnknnknnkara |
| 423 | nnkwatnnknnknnknnknnknnkara |
| 424 | nnkwatnnknnknnknnknnknnknnkara |
| 425 | nnkwatnnknnknnknnknnknnknnknnkara |
| 426 | nnkwatnnknnknnknnknnknnknnknnknnkara |

One advantage of a randomized antigen polypeptide is that a single nucleic acid with a degenerate base code can potentially express a large amount of different randomized antigen polypeptides, which increases the chances that any one screening experiment will identify one or more randomized antigen polypeptides that interact with a certain TCR. In some embodiments, the nucleic acid that encodes the randomized antigen polypeptide can encode at least 1×10⁴, at least 1×10⁵, at least 1×10⁶, at least 1×10⁷, at least 1×10⁸, at least 1×10⁹, at least 1×10¹⁰, at least 1×10¹¹, at least 1×10¹², at least 1×10¹³, at least 1×10¹⁴, or at least 1×10¹⁵ different randomized polypeptide antigens.

Peptide antigens that bind in the binding cleft of an HLA molecule are generally of a restricted length range. The majority of polypeptides that bind to class I HLA molecules are 8, 9, 10, or 11 amino acids in length. In some embodiments, the randomized antigen polypeptide which binds to an HLA molecule and forming the HLA-antigen polypeptide complexes of the present disclosure is between 8 and 11 amino acids in length. In some embodiments, the randomized antigen polypeptide is between 8 and 10 amino acids in length. In some embodiments, the randomized antigen polypeptide is 8 amino acids in length. In some embodiments, the randomized antigen polypeptide is 9 amino acids in length. In some embodiments, the randomized antigen polypeptide is 10 amino acids in length. In some embodiments, the randomized antigen polypeptide is 11 amino acids in length.

Another constituent of the HLA-antigen polypeptide complexes described herein is an HLA molecule, such as an HLA polypeptide. For the purposes of the current disclosure, the HLA molecule is a class I major histocompatibility molecule. In some embodiments, the plurality of HLA polypeptides of the HLA-antigen polypeptide complexes of the current disclosure (HLA-antigen complexes) can comprise any of the following loci and alleles: A3, A11, A23, A24, A26, A30, A31, A33, A68, B7, B8, B15, B27, B40, B44, B51, B53, C1, C2, C3, C4, C5, C6, C7, C8, and E. In some embodiments, each of the HLA-antigen complexes in the plurality of HLA-antigen complexes comprise an HLA polypeptide selected from the group of HLA polypeptides consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises at least five, ten, fifteen, twenty, or twenty-five different HLA polypeptides selected from the group of HLA polypeptides consisting of A3, A11, A23, A24, A26, A30, A31, A33, A68, B7, B8, B15, B27, B40, B44, B51, B53, C1, C2, C3, C4, C5, C6, C7, C8, and E. In some embodiments, the plurality of HLA-antigen complexes comprises all of the HLA polypeptides in the group of HLA polypeptides consisting of A3, A11, A23, A24, A26, A30, A31, A33, A68, B7, B8, B15, B27, B40, B44, B51, B53, C1, C2, C3, C4, C5, C6, C7, C8, and E.

In some embodiments, the amino acid sequence of the HLA polypeptide of the HLA-antigen polypeptide complex can comprise any of the amino acid sequences set forth in Table 6. In some embodiments, the HLA polypeptide comprises an amino acid sequence that is at least about 70%, 75%, 80%, 85%, 87%, 87.5%, 90%, 95%, 97%, 97.5%, 98%, 98.5%, 99%, 99.5%, or 100% identical to any one of, but not limited to, the amino acid sequences set forth in any one of SEQ ID NOs: 427 to 455. In some embodiments, the HLA polypeptide comprises an amino acid sequence identical to any one of those set forth in any one of SEQ ID NOs: 427 to 455. In some embodiments, a portion of the HLA polypeptide that comprises the peptide binding cleft is identical to any one of SEQ ID Nos: 251 to 279, and the non-peptide binding cleft residues are at least about 70%, 75%, 80%, 85%, 87.5%, 90%, 95%, 97%, 98%, 99%, or 100% identical to any one of SEQ ID NOs: 427 to 455. Also envisioned within the present disclosure are HLA polypeptide truncations that have 1, 2, 3, 4, 5, 6, 7, 8, 9, 10, 15, 20, or 25 amino acids truncated from the N-terminus or truncated from the C-terminus of any one of SEQ ID NOs: 427 to 455.

TABLE 6
HLA Allele Amino Acid Sequences

| SEQ ID NO: | Amino Acid Sequence |
|---|---|
| 427 | GSHSMRYFFTSVSRPGRGEPRFIAVGYVDDTQFVRFDSDAASQKMEPRAPWIEQEGPEYWDQETRNMKAH |
| | SQTDRANLGTLRGAYNQSEDGSHTIQIMYGCDVGPDGRFLRGYRQDAYDGKDYIALNEDLRSWTAADMAA |
| | QITKRKWEAVHAAEQRRVYLEGRCVDGLRRYLENGKETLQRTDPPKTHMTHHPISDHEATLRCWALGFYP |
| | AEITLTWQRDGEDQTQDTELVETRPAGDGTFQKWAAVVVPSGEEQRYTCHVQHEGLPKPLTLRWELSS |
| 428 | GSHSMRYFFTSVSRPGRGEPRFIAVGYVDDTQFVRFDSDAASQRMEPRAPWIEQEGPEYWDQETRNVKAQ |
| | SQTDRVDLGTLRGAYNQSEAGSHTIQIMYGCDVGSDGRFLRGYRQDAYDGKDYIALNEDLRSWTAADMAA |
| | QITKRKWEAAHEAEQLRAYLDGTCVEWLRRYLENGKETLQRTDPPKTHMTHHPISDHEATLRCWALGFYP |
| | AEITLTWQRDGEDQTQDTELVETRPAGDGTFQKWAAVVVPSGEEQRYTCHVQHEGLPKPLTLRWELSS |
| 429 | GSHSMRYFYTSVSRPGRGEPRFIAVGYVDDTQFVRFDSDAASQRMEPRAPWIEQEGPEYWDQETRNVKAQ |
| | SQTDRVDLGTLRGAYNQSEDGSHTIQEVIYGCDVGPDGRFLRGYRQDAYDGKDYIALNEDLRSWTAADMA |
| | AQITKRKWEAAHAAEQQRAYLEGRCVEWLRRYLENGKETLQRTDPPKTHMTHHPISDHEATLRCWALGEY |
| | PAEITLTWQRDGEDQTQDTELVETRPAGDGTFQKWAAVVVPSGEEQRYTCHVQHEGLPKPLTLRWELSS |
| 430 | GSHSMRYFSTSVSRPGRGEPRFIAVGYVDDTQFVREDSDAASQRMEPRAPWIEQEGPEYWDEETGKVKAH |
| | SQTDRENLRIALRAYNQSEAGSHTLQMMFGCDVGSDGRFLRGYHQYAYDGKDYIALKEDLRSWTAADMAA |
| | QITQRKWEAARVAEQLRAYLEGTCVDGLRRYLENGKETLQRTDPPKTHMTHHPISDHEATLRCWALGFYP |
| | AEITLTWQRDGEDQTQDTELVETRPAGDGTFQKWAAVVVPSGEEQRYTCHVQHEGLPKPLTLRWEPSS |
| 431 | GSHSMRYFSTSVSRPGRGEPRFIAVGYVDDTQFVREDSDAASQRMEPRAPWIEQEGPEYWDEETGKVKAH |
| | SQTDRENLRIALRAYNQSEAGSHTLQMMFGCDVGSDGRFLRGYHQYAYDGKDYIALKEDLRSWTAADMAA |
| | QITKRKWEAAHVAEQQRAYLEGTCVDGLRRYLENGKETLQRTDPPKTHMTHHPISDHEATLRCWALGEYP |
| | AEITLTWQRDGEDQTQDTELVETRPAGDGTFQKWAAVVVPSGEEQRYTCHVQHEGLPKPLTLRWEPSS |
| 432 | GSHSMRYFSTSVSRPGSGEPRFIAVGYVDDTQFVREDSDAASQRMEPRAPWIEQERPEYWDQETRNVKAQ |
| | SQTDRVDLGTLRGAYNQSEAGSHTIQIMYGCDVGSDGRFLRGYEQHAYDGKDYIALNEDLRSWTAADMAA |
| | QITQRKWEAARWAEQLRAYLEGTCVEWLRRYLENGKETLQRTDPPKTHMTHHPISDHEATLRCWALGEYP |
| | AEITLTWQRDGEDQTQDTELVETRPAGDGTFQKWAAVVVPSGEEQRYTCHVQHEGLPKPLTLRWELSS |
| 433 | GSHSMRYFTTSVSRPGRGEPRFIAVGYVDDTQFVRFDSDAASQRMEPRAPWIEQERPEYWDQETRNVKAH |
| | SQIDRVDLGTLRGAYNQSEAGSHTIQMMYGCDVGSDGRFLRGYQQDAYDGKDYIALNEDLRSWTAADMAA |
| | QITQRKWEAARVAEQLRAYLEGTCVEWLRRYLENGKETLQRTDPPKTHMTHHAVSDHEATLRCWALSFYP |
| | AEITLTWQRDGEDQTQDTELVETRPAGDGTFQKWASVVVPSGQEQRYTCHVQHEGLPKPLTLRWEPSS |
| 434 | GSHSMRYFTTSVSRPGRGEPRFIAVGYVDDTQFVRFDSDAASQRMEPRAPWIEQEGPEYWDRNTRNVKAH |
| | SQIDRVDLGTLRGAYNQSEAGSHTIQMMYGCDVGSDGRFLRGYQQDAYDGKDYIALNEDLRSWTAADMAA |
| | QITQRKWEAARVAEQLRAYLEGTCVEWLRRYLENGKETLQRTDPPKTHMTHHAVSDHEATLRCWALSFYP |
| | AEITLTWQRDGEDQTQDTELVETRPAGDGTFQKWASVVVPSGQEQRYTCHVQHEGLPKPLTLRWEPSS |
| 435 | GSHSMRYFYTSMSRPGRGEPRFIAVGYVDDTQFVREDSDAASQRMEPRAPWIEQEGPEYWDRNTRNVKAQ |
| | SQTDRVDLGTLRGAYNQSEAGSHTIQRMYGCDVGPDGRFLRGYHQYAYDGKDYIALKEDLRSWTAADMAA |
| | QTTKHKWEAAHVAEQWRAYLEGTCVEWLRRYLENGKETLQRTDAPKTHMTHHAVSDHEATLRCWALSFYP |
| | AEITLTWQRDGEDQTQDTELVETRPAGDGTFQKWVAVVVPSGQEQRYTCHVQHEGLPKPLTLRWEPSS |
| 436 | GSHSMRYFYTSVSRPGRGEPRFISVGYVDDTQFVREDSDAASPREEPRAPWIEQEGPEYWDRNTQIYKAQ |
| | AQTDRESLRNLRGAYNQSEAGSHTLQSMYGCDVGPDGRLLRGHDQYAYDGKDYIALNEDLRSWTAADTAA |
| | QITQRKWEAAREAEQRRAYLEGECVEWLRRYLENGKDKLERADPPKTHVTHHPISDHEATLRCWALGEYP |
| | AEITLTWQRDGEDQTQDTELVETRPAGDRTFQKWAAVVVPSGEEQRYTCHVQHEGLPKPLTLRWEPSS |
| 437 | GSHSMRYFDTAMSRPGRGEPRFISVGYVDDTQFVREDSDAASPREEPRAPWIEQEGPEYWDRNTQIFKTN |
| | TQTDRESLRNLRGAYNQSEAGSHTLQSMYGCDVGPDGRLLRGHNQYAYDGKDYIALNEDLRSWTAADTAA |
| | QITQRKWEAARVAEQDRAYLEGTCVEWLRRYLENGKDTLERADPPKTHVTHHPISDHEATLRCWALGFYP |
| | AEITLTWQRDGEDQTQDTELVETRPAGDRTFQKWAAVVVPSGEEQRYTCHVQHEGLPKPLTLRWEPSS |
| 438 | GSHSMRYFYTAMSRPGRGEPRFIAVGYVDDTQFVRFDSDAASPRMAPRAPWIEQEGPEYWDRETQISKTN |
| | TQTYRESLRNLRGAYNQSEAGSHTLQRMYGCDVGPDGRLLRGHDQSAYDGKDYIALNEDLSSWTAADTAA |
| | QITQRKWEAAREAEQWRAYLEGLCVEWLRRYLENGKETLQRADPPKTHVTHHPISDHEATLRCWALGEYP |
| | AEITLTWQRDGEDQTQDTELVETRPAGDRTFQKWAAVVVPSGEEQRYTCHVQHEGLPKPLTLRWEPSS |
| 439 | GSHSMRYFHTAMSRPGRGEPRFITVGYVDDTLFVRFDSDATSPRKEPRAPWIEQEGPEYWDRETQISKTN |
| | TQTYRESLRNLRGAYNQSEAGSHTLQRMYGCDVGPDGRLLRGHNQYAYDGKDYIALNEDLRSWTAADTAA |
| | QISQRKLEAARVAEQLRAYLEGECVEWLRRYLENGKDKLERADPPKTHVTHHPISDHEATLRCWALGFYP |
| | AEITLTWQRDGEDQTQDTELVETRPAGDRTFQKWAAVVVPSGEEQRYTCHVQHEGLPKPLTLRWEPSS |
| 440 | GSHSMRYFHTSVSRPGRGEPRFITVGYVDDTLFVRFDSDATSPRKEPRAPWIEQEGPEYWDRETQISKTN |
| | TQTYRESLRNLRGAYNQSEAGSHTLQSMYGCDVGPDGRLLRGHNQYAYDGKDYIALNEDLRSWTAADTAA |
| | QITQRKWEAARVAEQLRAYLEGECVEWLRRYLENGKETLQRADPPKTHVTHHPISDHEATLRCWALGFYP |
| | AEITLTWQRDGEDQTQDTELVETRPAGDRTFQKWAAVVVPSGEEQRYTCHVQHEGLPKPLTLRWEPSS |
| 441 | GSHSMRYFYTAMSRPGRGEPRFITVGYVDDTLFVRFDSDATSPRKEPRAPWIEQEGPEYWDRETQISKTN |
| | TQTYRENLRTALRAYNQSEAGSHIIQRMYGCDVGPDGRLLRGYDQDAYDGKDYIALNEDLSSWTAADTAA |
| | QITQRKWEAARVAEQDRAYLEGLCVESLRRYLENGKETLQRADPPKTHVTHHPISDHEVTLRCWALGEYP |
| | AEITLTWQRDGEDQTQDTELVETRPAGDRTFQKWAAVVVPSGEEQRYTCHVQHEGLPKPLTLRWEPSS |
| 442 | GSHSMRYFYTAMSRPGRGEPRFITVGYVDDTLFVRFDSDATSPRKEPRAPWIEQEGPEYWDRETQISKTN |
| | TQTYRENLRTALRAYNQSEAGSHIIQRMYGCDVGPDGRLLRGYDQDAYDGKDYIALNEDLSSWTAADTAA |
| | QITQRKWEAARVAEQLRAYLEGLCVESLRRYLENGKETLQRADPPKTHVTHHPISDHEVTLRCWALGEYP |
| | AEITLTWQRDGEDQTQDTELVETRPAGDRTFQKWAAVVVPSGEEQRYTCHVQHEGLPKPLTLRWEPSS |
| 443 | GSHSMRYFYTAMSRPGRGEPRFIAVGYVDDTQFVRFDSDAASPRTEPRAPWIEQEGPEYWDRNTQIFKTN |
| | TQTYRENLRIALRAYNQSEAGSHTWQTMYGCDVGPDGRLLRGHNQYAYDGKDYIALNEDLSSWTAADTAA |
| | QITQRKWEAAREAEQLRAYLEGLCVEWLRRHLENGKETLQRADPPKTHVTHHPVSDHEATLRCWALGFYP |
| | AEITLTWQRDGEDQTQDTELVETRPAGDRTFQKWAAVVVPSGEEQRYTCHVQHEGLPKPLTLRWEPSS |
| 444 | GSHSMRYFYTAMSRPGRGEPRFIAVGYVDDTQFVRFDSDAASPRTEPRAPWIEQEGPEYWDRNTQIFKTN |
| | TQTYRENLRIALRAYNQSEAGSHIIQRMYGCDLGPDGRLLRGHDQSAYDGKDYIALNEDLSSWTAADTAA |
| | QITQRKWEAARVAEQLRAYLEGLCVEWLRRYLENGKETLQRADPPKTHVTHHPVSDHEATLRCWALGFYP |
| | AEITLTWQRDGEDQTQDTELVETRPAGDRTFQKWAAVVVPSGEEQRYTCHVQHEGLPKPLTLRWEPSS |
| 445 | GSHSMRYFYTAMSRPGRGEPRFIAVGYVDDTQFVRFDSDAASPRTEPRAPWIEQEGPEYWDGETRNMKAS |
| | AQTYRENLRIALRAYNQSEAGSHIIQRMYGCDLGPDGRLLRGHDQSAYDGKDYIALNEDLSSWTAADTAA |
| | QITQRKWEAARVAEQLRAYLEGLCVEWLRRYLENGKETLQRADPPKTHVTHHPVSDHEATLRCWALGFYP |
| | AEITLTWQRDGEDQTQDTELVETRPAGDRTFQKWAAVVVPSGEEQRYTCHVQHEGLPKPLTLRWEPSS |
| 446 | CSHSMKYFFTSVSRPGRGEPRFISVGYVDDTQFVREDSDAASPRGEPRAPWVEQEGPEYWDRETQKYKRQ |
| | AQTDRVSLRNLRGAYNQSEAGSHTLQWMCGCDLGPDGRLLRGYDQYAYDGKDYIALNEDLRSWTAADTAA |
| | QITQRKWEAAREAEQRRAYLEGTCVEWLRRYLENGKETLQRAEHPKTHVTHHPVSDHEATLRCWALGEYP |
| | AEITLTWQWDGEDQTQDTELVETRPAGDGTFQKWAAVMVPSGEEQRYTCHVQHEGLPEPLTLRWEPSS |
| 447 | CSHSMRYFYTAVSRPSRGEPHFIAVGYVDDTQFVRFDSDAASPRGEPRAPWVEQEGPEYWDRETQKYKRQ |
| | AQTDRVNLRKLRGAYNQSEAGSHTLQRMYGCDLGPDGRLLRGYDQSAYDGKDYIALNEDLRSWTAADTAA |
| | QITQRKWEAAREAEQWRAYLEGECVEWLRRYLENGKETLQRAEHPKTHVTHHPVSDHEATLRCWALGFYP |
| | TEITLTWQRDGEDQTQDTELVETRPAGDGTFQKWAAVVVPSGEEQRYTCHVQHEGLPEPLTLRWEPSS |
| 448 | GSHSMRYFYTAVSRPGRGEPHFIAVGYVDDTQFVRFDSDAASPRGEPRAPWVEQEGPEYWDRETQKYKRQ |
| | AQTDRVSLRNLRGAYNQSEAGSHIIQRMYGCDVGPDGRLLRGYDQYAYDGKDYIALNEDLRSWTAADTAA |
| | QITQRKWEAAREAEQLRAYLEGLCVEWLRRYLKNGKETLQRAEHPKTHVTHHPVSDHEATLRCWALGEYP |
| | AEITLTWQWDGEDQTQDTELVETRPAGDGTFQKWAAVVVPSGEEQRYTCHVQHEGLPEPLTLRWEPSS |
| 449 | GSHSMRYFSTSVSWPGRGEPRFIAVGYVDDTQFVREDSDAASPRGEPREPWVEQEGPEYWDRETQKYKRQ |
| | AQADRVNLRKLRGAYNQSEDGSHTLQRMEGCDLGPDGRLLRGYNQFAYDGKDYIALNEDLRSWTAADTAA |
| | QITQRKWEAAREAEQRRAYLEGTCVEWLRRYLENGKETLQRAEHPKTHVTHHPVSDHEATLRCWALGEYP |
| | AEITLTWQWDGEDQTQDTELVETRPAGDGTFQKWAAVVVPSGEEQRYTCHVQHEGLPEPLTLRWKPSS |
| 450 | CSHSMRYFYTAVSRPGRGEPRFIAVGYVDDTQFVQFDSDAASPRGEPRAPWVEQEGPEYWDRETQKYKRQ |
| | AQTDRVNLRKLRGAYNQSEAGSHTLQRMYGCDLGPDGRLLRGYNQFAYDGKDYIALNEDLRSWTAADKAA |
| | QITQRKWEAAREAEQRRAYLEGTCVEWLRRYLENGKKTLQRAEHPKTHVTHHPVSDHEATLRCWALGEYP |
| | AEITLTWQRDGEDQTQDTELVETRPAGDGTFQKWAAVVVPSGEEQRYTCHVQHEGLPEPLTLRWGPSS |
| 451 | CSHSMRYFDTAVSRPGRGEPRFISVGYVDDTQFVREDSDAASPRGEPRAPWVEQEGPEYWDRETQKYKRQ |
| | AQADRVNLRKLRGAYNQSEDGSHTLQWMYGCDLGPDGRLLRGYDQSAYDGKDYIALNEDLRSWTAADTAA |
| | QITQRKWEAAREAEQWRAYLEGTCVEWLRRYLENGKETLQRAEHPKTHVTHHPVSDHEATLRCWALGFYP |
| | AEITLTWQRDGEDQTQDTELVETRPAGDGTFQKWAAVVVPSGEEQRYTCHVQHEGLPEPLTLRWEPSS |
| 452 | CSHSMRYFDTAVSRPGRGEPRFISVGYVDDTQFVREDSDAASPRGEPRAPWVEQEGPEYWDRETQNYKRQ |
| | AQADRVSLRNLRGAYNQSEDGSHTLQRMYGCDLGPDGRLLRGYDQSAYDGKDYIALNEDLRSWTAADTAA |
| | QITQRKLEAARAAEQLRAYLEGTCVEWLRRYLENGKETLQRAEPPKTHVTHHPLSDHEATLRCWALGEYP |
| | AEITLTWQRDGEDQTQDTELVETRPAGDGTFQKWAAVVVPSGQEQRYTCHMQHEGLQEPLTLSWEPSS |
| 453 | CSHSMRYFDTAVSRPGRGEPRFISVGYVDDTQFVREDSDAASPRGEPRAPWVEQEGPEYWDRETQKYKRQ |
| | AQADRVSLRNLRGAYNQSEDGSHTLQRMSGCDLGPDGRLLRGYDQSAYDGKDYIALNEDLRSWTAADTAA |
| | QITQRKLEAARAAEQLRAYLEGTCVEWLRRYLENGKETLQRAEPPKTHVTHHPLSDHEATLRCWALGEYP |
| | AEITLTWQRDGEDQTQDTELVETRPAGDGTFQKWAAVVVPSGQEQRYTCHMQHEGLQEPLTLSWEPSS |
| 454 | CSHSMRYFYTAVSRPGRGEPRFIAVGYVDDTQFVREDSDAASPRGEPRAPWVEQEGPEYWDRETQKYKRQ |
| | AQTDRVSLRNLRGAYNQSEAGSHTLQWMYGCDLGPDGRLLRGYDQSAYDGKDYIALNEDLRSWTAADTAA |
| | QITQRKWEAARAAEQQRAYLEGTCVEWLRRYLENGKETLQRAEHPKTHVTHHLVSDHEATLRCWALGEYP |
| | AEITLTWQRDGEDQTQDTELVETRPAGDGTFQKWAAVVVPSGEEQRYTCHVQHEGLPEPLTLRWEPSS |
| 455 | GSHSLKYFHTSVSRPGRGEPRFISVGYVDDTQFVRFDNDAASPRMVPRAPWMEQEGSEYWDRETRSARDT |
| | AQIFRVNLRTLRGAYNQSEAGSHTLQWMHGCELGPDGRFLRGYEQFAYDGKDYLTLNEDLRSWTAVDTAA |
| | QISEQKSNDASEAEHQRAYLEDTCVEWLHKYLEKGKETLLHLEPPKTHVTHHPISDHEATLRCWALGEYP |
| | AEITLTWQQDGEGHTQDTELVETRPAGDGTFQKWAAVVVPSGEEQRYTCHVQHEGLPEPVTLRWKPAS |

The HLA polypeptide of the HLA-antigen polypeptide complex can be encoded by a nucleic acid of any set forth in Table 7. In some embodiments, the HLA polypeptide is encoded by a nucleic acid sequence that is at least about 90%, 95%, 97%, 98%, 99%, or 100% homologous to at least, but not limited to, any one of the nucleic acid sequences listed in Table 7, such as SEQ ID NOs: 456 to 484. In some embodiments, the HLA polypeptide is encoded by a nucleic acid sequence identical to that set forth in any one of SEQ ID NOs: 456 to 484.

TABLE 7
HLA Allele Nucleic Acid Sequences

| SEQ ID NO: | DNA sequence |
|---|---|
| 456 | ggctcccactccatgaggtatttcttcacatccgtgtcccggcccggccgcggggagccccgc |
| | ttcatcgccgtgggctacgtggacgacacgcagttcgtgcggttcgacagcgacgccgcgagc |
| | cagaagatggagccgcgggcgccgtggatagagcaggaggggccggagtattgggaccaggag |
| | acacggaatatgaaggcccactcacagactgaccgagcgaacctggggaccdgcgcggcgcct |
| | acaaccagagcgaggacggttctcacaccatccagataatgtatggctgcgacgtggggccgg |
| | acgggcgcttcctccgcgggtaccggcaggacgcctacgacggcaaggattacatcgccctga |
| | acgaggacctgcgctcttggaccgcggcggacatggcagctcagatcaccaagcgcaagtggg |
| | aggcggtccatgcggcggagcagcggagagtctacctggagggccggtgcgtggacgggctcc |
| | gcagatacctggagaacgggaaggagacactgcagcgcacggacccccccaagacacatatga |
| | cccaccaccccatctctgaccatgaggccaccctgaggtgctgggccctgggcttctaccctg |
| | cggagatcacactgacctggcagcgggatggggaggaccagacccaggacacggagctcgtgg |
| | agaccaggcctgcaggggatggaaccttccagaagtgggcggctgtggtggtgccttctggag |
| | aggagcagagatacacctgccatgtgcagcatgagggtctgcccaagcccctcaccctgagat |
| | gggagctgtcttcc |
| 457 | ggctcccactccatgaggtatttcttcacatccgtgtcccggcccggccgcggggagccccgc |
| | ttcatcgccgtgggctacgtggacgacacgcagttcgtgcggttcgacagcgacgccgcgagc |
| | cagaggatggagccgcgggcgccgtggatagagcaggaggggccggagtattgggaccaggag |
| | acacggaatgtgaaggcccagtcacagactgaccgagtggacctggggaccctgcgcggcgcc |
| | tacaaccagagcgaggccggttctcacaccatccagataatgtatggctgcgacgtggggtcg |
| | gacgggcgcttcctccgcgggtaccggcaggacgcctacgacggcaaggattacatcgccctg |
| | aacgaggacctgcgctcttggaccgcggcggacatggcggctcagatcaccaagcgcaagtgg |
| | gaggcggcccatgaggcggagcagttgagagcctacctggatggcacgtgcgtggagtggctc |
| | cgcagatacctggagaacgggaaggagacactgcagcgcacggacccccccaagacacatatg |
| | acccaccaccccatctctgaccatgaggccaccctgaggtgctgggccctgggcttctaccct |
| | gcggagatcacactgacctggcagcgggatggggaggaccagacccaggacacggagctcgtg |
| | gagaccaggcctgcaggggatggaaccttccagaagtgggcggctgtggtggtgccttctgga |
| | gaggagcagagatacacctgccatgtgcagcatgagggtctgcccaagcccctcaccctgaga |
| | tgggagctgtcttcc |
| 458 | ggctcccactccatgaggtatttctacacctccgtgtcccggcccggccgcggggagccccgc |
| | ttcatcgccgtgggctacgtggacgacacgcagttcgtgcggttcgacagcgacgccgcgagc |
| | cagaggatggagccgcgggcgccgtggatagagcaggaggggccggagtattgggaccaggag |
| | acacggaatgtgaaggcccagtcacagactgaccgagtggacctggggaccctgcgcggcgcc |
| | tacaaccagagcgaggacggttctcacaccatccagataatgtatggctgcgacgtggggccg |
| | gacgggcgcttcctccgcgggtaccggcaggacgcctacgacggcaaggattacatcgccctg |
| | aacgaggacctgcgctcttggaccgcggcggacatggcagctcagatcaccaagcgcaagtgg |
| | gaggcggcccatgcggcggagcagcagagagcctacctggagggccggtgcgtggagtggctc |
| | cgcagatacctggagaacgggaaggagacactgcagcgcacggacccccccaagacacatatg |
| | acccaccaccccatctctgaccatgaggccaccctgaggtgctgggccctgggcttctaccct |
| | gcggagatcacactgacctggcagcgggatggggaggaccagacccaggacacggagctcgtg |
| | gagaccaggcctgcaggggatggaaccttccagaagtgggcggctgtggtggtgccttctgga |
| | gaggagcagagatacacctgccatgtgcagcatgagggtctgcccaagcccctcaccctgaga |
| | tgggagctgtcttcc |
| 459 | ggctcccactccatgaggtatttctccacatccgtgtcccggcccggccgcggggagccccgc |
| | ttcatcgccgtgggctacgtggacgacacgcagttcgtgcggttcgacagcgacgccgcgagc |
| | cagaggatggagccgcgggcgccgtggatagagcaggaggggccggagtattgggacgaggag |
| | acagggaaagtgaaggcccactcacagactgaccgagagaacctgcggatcgcgctccgcgcc |
| | tacaaccagagcgaggccggttctcacaccctccagatgatgtttggctgcgacgtggggtcg |
| | gacgggcgcttcctccgcgggtaccaccagtacgcctacgacggcaaggattacatcgccctg |
| | aaagaggacctgcgctcttggaccgcggcggacatggcggctcagatcacccagcgcaagtgg |
| | gaggcggcccgtgtggcggagcagttgagagcctacctggagggcacgtgcgtggacgggctc |
| | cgcagatacctggagaacgggaaggagacactgcagcgcacggacccccccaagacacatatg |
| | acccaccaccccatctctgaccatgaggccactctgagatgctgggccctgggcttctaccct |
| | gcggagatcacactgacctggcagcgggatggggaggaccagacccaggacacggagcttgtg |
| | gagaccaggcctgcaggggatggaaccttccagaagtgggcagctgtggtggtaccttctgga |
| | gaggagcagagatacacctgccatgtgcagcatgagggtctgcccaagcccctcaccctgaga |
| | tgggagccatcttcc |
| 460 | ggctcccactccatgaggtatttctccacatccgtgtcccggcccggccgcggggagccccgc |
| | ttcatcgccgtgggctacgtggacgacacgcagttcgtgcggttcgacagcgacgccgcgagc |
| | cagaggatggagccgcgggcgccgtggatagagcaggaggggccggagtattgggacgaggag |
| | acagggaaagtgaaggcccactcacagactgaccgagagaacctgcggatcgcgctccgcgcc |
| | tacaaccagagcgaggccggttctcacaccctccagatgatgtttggctgcgacgtggggtcg |
| | gacgggcgcttcctccgcgggtaccaccagtacgcctacgacggcaaggattacatcgccctg |
| | aaagaggacctgcgctcttggaccgcggcggacatggcggctcagatcaccaagcgcaagtgg |
| | gaggcggcccatgtggcggagcagcagagagcctacctggagggcacgtgcgtggacgggctc |
| | cgcagatacctggagaacgggaaggagacactgcagcgcacggacccccccaagacacatatg |
| | acccaccaccccatctctgaccatgaggccactctgagatgctgggccctgggcttctaccct |
| | gcggagatcacactgacctggcagcgggatggggaggaccagacccaggacacggagcttgtg |
| | gagaccaggcctgcaggggatggaaccttccagaagtgggcagctgtggtggtaccttctgga |
| | gaggagcagagatacacctgccatgtgcagcatgagggtctgcccaagcccctcaccctgaga |
| | tgggagccatcttcc |
| 461 | ggctcccactccatgaggtatttctccacatccgtgtcccggcccggcagtggagagccccgc |
| | ttcatcgcagtgggctacgtggacgacacgcagttcgtgcggttcgacagcgacgccgcgagc |
| | cagaggatggagccgcgggcgccgtggatagagcaggagaggcctgagtattgggaccaggag |
| | acacggaatgtgaaggcccagtcacagactgaccgagtggacctggggaccctgcgcggcgcc |
| | tacaaccagagcgaggccggttctcacaccatccagataatgtatggctgcgacgtggggtcg |
| | gacgggcgcttcctccgcgggtatgaacagcacgcctacgacggcaaggattacatcgccctg |
| | aacgaggacctgcgctcttggaccgcggcggacatggcggctcagatcacccagcgcaagtgg |
| | gaggcggcccgttgggcggagcagttgagagcctacctggagggcacgtgcgtggagtggctc |
| | cgcagatacctggagaacgggaaggagacactgcagcgcacggacccccccaagacacatatg |
| | acccaccaccccatctctgaccatgaggccaccctgaggtgctgggccctgggcttctaccct |
| | gcggagatcacactgacctggcagcgggatggggaggaccagacccaggacacggagctcgtg |
| | gagaccaggcctgcaggggatggaaccttccagaagtgggcggctgtggtggtgccttctgga |
| | gaggagcagagatacacctgccatgtgcagcatgagggtctgcccaagcccctcaccctgaga |
| | tgggagctgtcttcc |
| 462 | ggctcccactccatgaggtatttcaccacatccgtgtcccggcccggccgcggggagccccgc |
| | ttcatcgccgtgggctacgtggacgacacgcagttcgtgcggttcgacagcgacgccgcgagc |
| | cagaggatggagccgcgggcgccgtggatagagcaggagaggcctgagtattgggaccaggag |
| | acacggaatgtgaaggcccactcacagattgaccgagtggacctggggaccctgcgcggcgcc |
| | tacaaccagagcgaggccggttctcacaccatccagatgatgtatggctgcgacgtggggtcg |
| | gacgggcgcttcctccgcgggtaccagcaggacgcctacgacggcaaggattacatcgccttg |
| | aacgaggacctgcgctcttggaccgcggcggacatggcggctcagatcacccagcgcaagtgg |
| | gaggcggcccgtgtggcggagcagttgagagcctacctggagggcacgtgcgtggagtggctc |
| | cgcagatacctggagaacgggaaggagacactgcagcgcacggacccccccaagacgcatatg |
| | actcaccacgctgtctctgaccatgaggccaccctgaggtgctgggccctgagcttctaccct |
| | gcggagatcacactgacctggcagcgggatggggaggaccagacccaggacacggagctcgtg |
| | gagaccaggcctgcaggggatggaaccttccagaagtgggcgtctgtggtggtgccttctgga |
| | caggagcagagatacacctgccatgtgcagcatgagggtctccccaagcccctcaccctgaga |
| | tgggagccgtcttcc |
| 463 | ggctcccactccatgaggtatttcaccacatccgtgtcccggcccggccgcggggagccccgc |
| | ttcatcgccgtgggctacgtggacgacacgcagttcgtgcggttcgacagcgacgccgcgagc |
| | cagaggatggagccgcgggcgccgtggatagagcaggaggggccggagtattgggaccggaac |
| | acacggaatgtgaaggcccactcacagattgaccgagtggacctggggaccctgcgcggcgcc |
| | tacaaccagagcgaggccggttctcacaccatccagatgatgtatggctgcgacgtggggtcg |
| | gacgggcgcttcctccgcgggtaccagcaggacgcctacgacggcaaggattacatcgccttg |
| | aacgaggacctgcgctcttggaccgcggcggacatggcggctcagatcacccagcgcaagtgg |
| | gaggcggcccgtgtggcggagcagttgagagcctacctggagggcacgtgcgtggagtggctc |
| | cgcagatacctggagaacgggaaggagacactgcagcgcacggacccccccaagacgcatatg |
| | actcaccacgctgtctctgaccatgaggccaccctgaggtgctgggccctgagcttctaccct |
| | gcggagatcacactgacctggcagcgggatggggaggaccagacccaggacacggagctcgtg |
| | gagaccaggcctgcaggggatggaaccttccagaagtgggcgtctgtggtggtgccttctgga |
| | caggagcagagatacacctgccatgtgcagcatgagggtctccccaagcccctcaccctgaga |
| | tgggagccgtcttcc |
| 464 | ggctcccactccatgaggtatttctacacctccatgtcccggcccggccgcggggagccccgc |
| | ttcatcgccgtgggctacgtggacgacacgcagttcgtgcggttcgacagcgacgccgcgagc |
| | cagaggatggagccgcgggcgccgtggatagagcaggaggggccggagtattgggaccggaac |
| | acacggaatgtgaaggcccagtcacagactgaccgagtggacctggggaccctgcgcggcgcc |
| | tacaaccagagcgaggccggttctcacaccatccagaggatgtatggctgcgacgtggggccg |
| | gacgggcgcttcctccgcgggtaccaccagtacgcctacgacggcaaggattacatcgccctg |
| | aaagaggacctgcgctcttggaccgcggcggacatggcagctcagaccaccaagcacaagtgg |
| | gaggcggcccatgtggcggagcagtggagagcctacctggagggcacgtgcgtggagtggctc |
| | cgcagatacctggagaacgggaaggagacactgcagcgcacggacgcccccaaaacgcatatg |
| | actcaccacgctgtctctgaccatgaagccaccctgaggtgctgggccctgagcttctaccct |
| | gcggagatcacactgacctggcagcgggatggggaggaccagacccaggacacggagctcgtg |
| | gagaccaggcctgcaggggatggaaccttccagaagtgggtggctgtggtggtgccttctgga |
| | caggagcagagatacacctgccatgtgcagcatgagggtttgcccaagcccctcaccctgaga |
| | tgggagccgtcttcc |
| 465 | ggctcccactccatgaggtatttctacacctccgtgtcccggcccggccgcggggagccccgc |
| | ttcatctcagtgggctacgtggacgacacccagttcgtgaggttcgacagcgacgccgcgagt |
| | ccgagagaggagccgcgggcgccgtggatagagcaggaggggccggagtattgggaccggaac |
| | acacagatctacaaggcccaggcacagactgaccgagagagcctgcggaacctgcgcggcgcc |
| | tacaaccagagcgaggccgggtctcacaccctccagagcatgtacggctgcgacgtggggccg |
| | gacgggcgcctcctccgcgggcatgaccagtacgcctacgacggcaaggattacatcgccctg |
| | aacgaggacctgcgctcctggaccgccgcggacacggcggctcagatcacccagcgcaagtgg |
| | gaggcggcccgtgaggcggagcagcggagagcctacctggagggcgagtgcgtggagtggctc |
| | cgcagatacctggagaacgggaaggacaagctggagcgcgctgaccccccaaagacacacgtg |
| | acccaccaccccatctctgaccatgaggccaccctgaggtgctgggccctgggtttctaccct |
| | gcggagatcacactgacctggcagcgggatggcgaggaccaaactcaggacactgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccgtcttcc |
| 466 | ggctcccactccatgaggtatttcgacaccgccatgtcccggcccggccgcggggagccccgc |
| | ttcatctcagtgggctacgtggacgacacgcagttcgtgaggttcgacagcgacgccgcgagt |
| | ccgagagaggagccgcgggcgccgtggatagagcaggaggggccggagtattgggaccggaac |
| | acacagatcttcaagaccaacacacagactgaccgagagagcctgcggaacctgcgcggcgcc |
| | tacaaccagagcgaggccgggtctcacaccctccagagcatgtacggctgcgacgtggggccg |
| | gacgggcgcctcctccgcgggcataaccagtacgcctacgacggcaaggattacatcgccctg |
| | aacgaggacctgcgctcctggaccgcggcggacaccgcggctcagatcacccagcgcaagtgg |
| | gaggcggcccgtgtggcggagcaggacagagcctacctggagggcacgtgcgtggagtggctc |
| | cgcagatacctggagaacgggaaggacacgctggagcgcgcggaccccccaaagacacacgtg |
| | acccaccaccccatctctgaccatgaggccaccctgaggtgctgggccctgggcttctaccct |
| | gcggagatcacactgacctggcagcgggatggcgaggaccaaactcaggacactgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccgtcttcc |
| 467 | ggctcccactccatgaggtatttctacaccgccatgtcccggcccggccgcggggagccccgc |
| | ttcatcgcagtgggctacgtggacgacacccagttcgtgaggttcgacagcgacgccgcgagt |
| | ccgaggatggcgccccgggcgccatggatagagcaggaggggccggagtattgggaccgggag |
| | acacagatctccaagaccaacacacagacttaccgagagagcctgcggaacctgcgcggcgcc |
| | tacaaccagagcgaggccgggtctcacaccctccagaggatgtacggctgcgacgtggggccg |
| | gacgggcgcctcctccgcgggcatgaccagtccgcctacgacggcaaggattacatcgccctg |
| | aacgaggacctgagctcctggaccgcggcggacacggcggctcagatcacccagcgcaagtgg |
| | gaggcggcccgtgaggcggagcagtggagagcctacctggagggcctgtgcgtggagtggctc |
| | cgcagatacctggagaacgggaaggagacactgcagcgcgcggaccccccaaagacacatgtg |
| | acccaccaccccatctctgaccatgaggccaccctgaggtgctgggccctgggcttctaccct |
| | gcggagatcacactgacctggcagcgggatggcgaggaccaaactcaggacaccgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccatcttcc |
| 468 | ggctcccactccatgaggtatttccacaccgccatgtcccggcccggccgcggggagccccgc |
| | ttcatcaccgtgggctacgtggacgacacgctgttcgtgaggttcgacagcgacgccacgagt |
| | ccgaggaaggagccgcgggcgccatggatagagcaggaggggccggagtattgggaccgggag |
| | acacagatctccaagaccaacacacagacttaccgagagagcctgcggaacctgcgcggcgcc |
| | tacaaccagagcgaggccgggtctcacaccctccagaggatgtacggctgcgacgtggggccg |
| | gacgggcgcctcctccgcgggcataaccagtacgcctacgacggcaaggattacatcgccctg |
| | aacgaggacctgcgctcctggaccgccgcggacacggcggctcagatctcccagcgcaagttg |
| | gaggcggcccgtgtggcggagcagctgagagcctacctggagggcgagtgcgtggagtggctc |
| | cgcagatacctggagaacgggaaggacaagctggagcgcgctgaccccccaaagacacacgtg |
| | acccaccaccccatctctgaccatgaggccaccctgaggtgctgggccctgggtttctaccct |
| | gcggagatcacactgacctggcagcgggatggcgaggaccaaactcaggacactgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccgtcttcc |
| 469 | ggctcccactccatgaggtatttccacacctccgtgtcccggcccggccgcggggagccccgc |
| | ttcatcaccgtgggctacgtggacgacacgctgttcgtgaggttcgacagcgacgccacgagt |
| | ccgaggaaggagccgcgggcgccatggatagagcaggaggggccggagtattgggaccgggag |
| | acacagatctccaagaccaacacacagacttaccgagagagcctgcggaacctgcgcggcgcc |
| | tacaaccagagcgaggccgggtctcacaccctccagagcatgtacggctgcgacgtggggccg |
| | gacgggcgcctcctccgcgggcataaccagtacgcctacgacggcaaggattacatcgccctg |
| | aacgaggacctgcgctcctggaccgccgcggacacggcggctcagatcacccagcgcaagtgg |
| | gaggcggcccgtgtggcggagcagctgagagcctacctggagggcgagtgcgtggagtggctc |
| | cgcagatacctggagaacgggaaggagacactgcagcgcgcggaccccccaaagacacacgtg |
| | acccaccaccccatctctgaccatgaggccaccctgaggtgctgggccctgggcttctaccct |
| | gcggagatcacactgacctggcagcgggatggcgaggaccaaactcaggacactgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccgtcttcc |
| 470 | ggctcccactccatgaggtatttctacaccgccatgtcccggcccggccgcggggagccccgc |
| | ttcatcaccgtgggctacgtggacgacacgctgttcgtgaggttcgacagcgacgccacgagt |
| | ccgaggaaggagccgcgggcgccatggatagagcaggaggggccggagtattgggaccgggag |
| | acacagatctccaagaccaacacacagacttaccgagagaacctgcgcaccgcgctccgcgcc |
| | tacaaccagagcgaggccgggtctcacatcatccagaggatgtacggctgcgacgtggggccg |
| | gacgggcgcctcctccgcgggtatgaccaggacgcctacgacggcaaggattacatcgccctg |
| | aacgaggacctgagctcctggaccgcggcggacaccgcggctcagatcacccagcgcaagtgg |
| | gaggcggcccgtgtggcggagcaggacagagcctacctggagggcctgtgcgtggagtcgctc |
| | cgcagatacctggagaacgggaaggagacactgcagcgcgcggaccccccaaagacacatgtg |
| | acccaccaccccatctctgaccatgaggtcaccctgaggtgctgggccctgggcttctaccct |
| | gcggagatcacactgacctggcagcgggatggcgaggaccaaactcaggacaccgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccgtcttcc |
| 471 | ggctcccactccatgaggtatttctacaccgccatgtcccggcccggccgcggggagccccgc |
| | ttcatcaccgtgggctacgtggacgacacgctgttcgtgaggttcgacagcgacgccacgagt |
| | ccgaggaaggagccgcgggcgccatggatagagcaggaggggccggagtattgggaccgggag |
| | acacagatctccaagaccaacacacagacttaccgagagaacctgcgcaccgcgctccgcgcc |
| | tacaaccagagcgaggccgggtctcacatcatccagaggatgtacggctgcgacgtggggccg |
| | gacgggcgcctcctccgcgggtatgaccaggacgcctacgacggcaaggattacatcgccctg |
| | aacgaggacctgagctcctggaccgcggcggacaccgcggctcagatcacccagcgcaagtgg |
| | gaggcggcccgtgtggcggagcagctgagagcctacctggagggcctgtgcgtggagtcgctc |
| | cgcagatacctggagaacgggaaggagacactgcagcgcgcggaccccccaaagacacatgtg |
| | acccaccaccccatctctgaccatgaggtcaccctgaggtgctgggccctgggcttctaccct |
| | gcggagatcacactgacctggcagcgggatggcgaggaccaaactcaggacaccgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccgtcttcc |
| 472 | ggctcccactccatgaggtatttctacaccgccatgtcccggcccggccgcggggagccccgc |
| | ttcattgcagtgggctacgtggacgacacccagttcgtgaggttcgacagcgacgccgcgagt |
| | ccgaggacggagccccgggcgccatggatagagcaggaggggccggagtattgggaccggaac |
| | acacagatcttcaagaccaacacacagacttaccgagagaacctgcggatcgcgctccgcgcc |
| | tacaaccagagcgaggccgggtctcacacttggcagacgatgtatggctgcgacgtggggccg |
| | gacgggcgcctcctccgcgggcataaccagtacgcctacgacggcaaagattacatcgccctg |
| | aacgaggacctgagctcctggaccgcggcggacaccgcggctcagatcacccagcgcaagtgg |
| | gaggcggcccgtgaggcggagcagctgagagcctacctggagggcctgtgcgtggagtggctc |
| | cgcagacacctggagaacgggaaggagacactgcagcgcgcggaccccccaaagacacacgtg |
| | acccaccacccagtctctgaccatgaggccaccctgaggtgctgggccctgggcttctaccct |
| | gcggagatcacactgacctggcagcgggatggcgaggaccaaactcaggacactgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccatcttcc |
| 473 | ggctcccactccatgaggtatttctacaccgccatgtcccggcccggccgcggggagccccgc |
| | ttcatcgcagtgggctacgtggacgacacccagttcgtgaggttcgacagcgacgccgcgagt |
| | ccgaggacggagccccgggcgccatggatagagcaggaggggccggagtattgggaccggaac |
| | acacagatcttcaagaccaacacacagacttaccgagagaacctgcggatcgcgctccgcgcc |
| | tacaaccagagcgaggccgggtctcacatcatccagaggatgtatggctgcgacctggggccc |
| | gacgggcgcctcctccgcgggcatgaccagtccgcctacgacggcaaggattacatcgccctg |
| | aacgaggacctgagctcctggaccgcggcggacaccgcggctcagatcacccagcgcaagtgg |
| | gaggcggcccgtgtggcggagcagctgagagcctacctggagggcctgtgcgtggagtggctc |
| | cgcagatacctggagaacgggaaggagacactgcagcgcgcggaccccccaaagacacacgtg |
| | acccaccacccagtctctgaccatgaggccaccctgaggtgctgggccctgggcttctaccct |
| | gcggagatcacactgacctggcagcgggatggcgaggaccaaactcaggacactgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccatcttcc |
| 474 | ggctcccactccatgaggtatttctacaccgccatgtcccggcccggccgcggggagccccgc |
| | ttcatcgcagtgggctacgtggacgacacccagttcgtgaggttcgacagcgacgccgcgagt |
| | ccgaggacggagccccgggcgccatggatagagcaggaggggccggagtattgggacggggag |
| | acacggaacatgaaggcctccgcgcagacttaccgagagaacctgcggatcgcgctccgcgcc |
| | tacaaccagagcgaggccgggtctcacatcatccagaggatgtatggctgcgacctggggccc |
| | gacgggcgcctcctccgcgggcatgaccagtccgcctacgacggcaaggattacatcgccctg |
| | aacgaggacctgagctcctggaccgcggcggacaccgcggctcagatcacccagcgcaagtgg |
| | gaggcggcccgtgtggcggagcagctgagagcctacctggagggcctgtgcgtggagtggctc |
| | cgcagatacctggagaacgggaaggagacactgcagcgcgcggaccccccaaagacacacgtg |
| | acccaccacccagtctctgaccatgaggccaccctgaggtgctgggccctgggcttctaccct |
| | gcggagatcacactgacctggcagcgggatggcgaggaccaaactcaggacactgagcttgtg |
| | gagaccagaccagcaggagatagaaccttccagaagtgggcagctgtggtggtgccttctgga |
| | gaagagcagagatacacatgccatgtacagcatgaggggctgccgaagcccctcaccctgaga |
| | tgggagccatcttcc |
| 475 | tgctcccactccatgaagtatttcttcacatccgtgtcccggcctggccgcggagagccccgc |
| | ttcatctcagtgggctacgtggacgacacgcagttcgtgcggttcgacagcgacgccgcgagt |
| | ccgagaggggagccgcgggcgccgtgggtggagcaggaggggccggagtattgggaccgggag |
| | acacagaagtacaagcgccaggcacagactgaccgagtgagcctgcggaacctgcgcggcgcc |
| | tacaaccagagcgaggccgggtctcacaccctccagtggatgtgtggctgcgacctggggccc |
| | gacgggcgcctcctccgcgggtatgaccagtacgcctacgacggcaaggattacatcgccctg |
| | aacgaggacctgcgctcctggaccgccgcggacaccgcggctcagatcacccagcgcaagtgg |
| | gaggcggcccgtgaggcggagcagcggagagcctacctggagggcacgtgcgtggagtggctc |
| | cgcagatacctggagaacgggaaggagacactgcagcgcgcggaacacccaaagacacacgtg |
| | acccaccatccagtctctgaccatgaggccaccctgaggtgctgggccctgggcttctaccct |
| | gcggagatcacactgacctggcagtgggatggggaggaccaaactcaggacaccgagcttgtg |
| | gagaccaggccagcaggagatggaaccttccagaagtgggcagctgtgatggtgccttctgga |
| | gaagagcagagatacacgtgccatgtgcagcacgaggggctgccggagcccctcaccctgaga |
| | tgggagccgtcttcc |
| 476 | tgctcccactccatgaggtatttctacaccgctgtgtcccggcccagccgcggagagccccac |
| | ttcatcgcagtgggctacgtggacgacacgcagttcgtgcggttcgacagcgacgccgcgagt |
| | ccaagaggggagccgcgggcgccgtgggtggagcaggaggggccggagtattgggaccgggag |
| | acacagaagtacaagcgccaggcacagactgaccgagtgaacctgcggaaactacgcggcgcc |
| | tacaaccagagcgaggccgggtctcacaccctccagaggatgtacggctgcgacctggggccc |
| | gacgggcgcctcctccgcgggtatgaccagtccgcctacgacggcaaggattacatcgccctg |
| | aacgaggacctgcgctcctggaccgccgcggacacagcggctcagatcacccagcgcaagtgg |
| | gaggcggcccgtgaggcggagcagtggagagcctacctggagggcgagtgcgtggagtggctc |
| | cgcagatacctggagaacgggaaggagacactgcagcgcgcggaacacccaaagacacacgtg |
| | acccaccatccagtctctgaccatgaggccaccctgaggtgctgggccctgggcttctaccct |
| | acggagatcacactgacctggcagcgggatggcgaggaccaaactcaggacaccgagcttgtg |
| | gagaccaggccagcaggagatggaaccttccagaagtgggcagctgtggtggtgccttctgga |
| | gaagagcagagatacacgtgccatgtgcagcacgaggggctgccggagcccctcaccctgaga |
| | tgggagccatcttcc |
| 477 | ggctcccactccatgaggtatttctacaccgctgtgtcccggcccggccgcggggagccccac |
| | ttcatcgcagtgggctacgtggacgacacgcagttcgtgcggttcgacagcgacgccgcgagt |
| | ccgagaggggagccgcgggcgccgtgggtggagcaggaggggccggagtattgggaccgggag |
| | acacagaagtacaagcgccaggcacagactgaccgagtgagcctgcggaacctgcgcggcgcc |
| | tacaaccagagcgaggccgggtctcacatcatccagaggatgtatggctgcgacgtggggccc |
| | gacgggcgcctcctccgcgggtatgaccagtacgcctacgacggcaaggattacatcgccctg |
| | aacgaggatctgcgctcctggaccgccgcggacacggcggctcagatcacccagcgcaagtgg |
| | gaggcggcccgtgaggcggagcagctgagagcctacctggagggcctgtgcgtggagtggctc |
| | cgcagatacctgaagaatgggaaggagacactgcagcgcgcggaacacccaaagacacacgtg |
| | acccaccatccagtctctgaccatgaggccaccctgaggtgctgggccctgggcttctaccct |
| | gcggagatcacactgacctggcagtgggatggggaggaccaaactcaggacactgagcttgtg |
| | gagaccaggccagcaggagatggaaccttccagaagtgggcagctgtggtggtgccttctgga |
| | gaagagcagagatacacgtgccatgtgcagcacgaggggctgccggagcccctcaccctgaga |
| | tgggagccgtcttcc |
| 478 | ggctcccactccatgaggtatttctccacatccgtgtcctggcccggccgcggggagccccgc |
| | ttcatcgcagtgggctacgtggacgacacgcagttcgtgcggttcgacagcgacgccgcgagt |
| | ccaagaggggagccgcgggagccgtgggtggagcaggaggggccggagtattgggaccgggag |
| | acacagaagtacaagcgccaggcacaggctgaccgagtgaacctgcggaaactgcgcggcgcc |
| | tacaaccagagcgaggacgggtctcacaccctccagaggatgtttggctgcgacctggggccg |
| | gacgggcgcctcctccgcgggtataaccagttcgcctacgacggcaaggattacatcgccctg |
| | aacgaggatctgcgctcctggaccgccgcggacacggcggctcagatcacccagcgcaagtgg |
| | gaggcggcccgtgaggcggagcagcggagagcctacctggagggcacgtgcgtggagtggctc |
| | cgcagatacctggagaacgggaaggagacactgcagcgcgcggaacacccaaagacacacgtg |
| | acccaccatccagtctctgaccatgaggccaccctgaggtgctgggccctgggcttctaccct |
| | gcggagatcacactgacctggcagtgggatggggaggaccaaactcaggacaccgagcttgtg |
| | gagaccaggccagcaggagatggaaccttccagaagtgggcagctgtggtggtgccttctgga |
| | gaagagcagagatacacgtgccatgttcagcacgaggggctgccggagcccctcaccctgaga |
| | tggaagccgtcttcc |
| 479 | tgctcccactccatgaggtatttctacaccgccgtgtcccggcccggccgcggagagccccgc |
| | ttcatcgcagtgggctacgtggacgacacgcagttcgtgcagttcgacagcgacgccgcgagt |
| | ccaagaggggagccgcgggcgccgtgggtggagcaggaggggccggagtattgggaccgggag |
| | acacagaagtacaagcgccaggcacagactgaccgagtgaacctgcggaaactgcgcggcgcc |
| | tacaaccagagcgaggccgggtctcacaccctccagaggatgtatggctgcgacctggggccc |
| | gacgggcgcctcctccgcgggtataaccagttcgcctacgacggcaaggattacatcgccctg |
| | aatgaggacctgcgctcctggaccgccgcggacaaggcggctcagatcacccagcgcaagtgg |
| | gaggcggcccgtgaggcggagcagcggagagcctacctggagggcacgtgcgtggagtggctc |
| | cgcagatacctggagaacgggaagaagacgctgcagcgcgcggaacacccaaagacacacgtg |
| | acccaccatccagtctctgaccatgaggccaccctgaggtgctgggccctgggcttctaccct |
| | gcggagatcacactgacctggcagcgggatggcgaggaccaaactcaggacaccgagcttgtg |
| | gagaccaggccagcaggagatggaaccttccagaagtgggcagctgtggtggtgccttctgga |
| | gaagagcagagatacacgtgccatgtgcagcacgaggggctgccagagcccctcaccctgaga |
| | tgggggccatcttcc |
| 480 | tgctcccactccatgaggtatttcgacaccgccgtgtcccggcccggccgcggagagccccgc |
| | ttcatctcagtgggctacgtggacgacacgcagttcgtgcggttcgacagcgacgccgcgagt |
| | ccgagaggggagccccgggcgccgtgggtggagcaggaggggccggagtattgggaccgggag |
| | acacagaagtacaagcgccaggcacaggctgaccgagtgaacctgcggaaactgcgcggcgcc |
| | tacaaccagagcgaggacgggtctcacaccctccagtggatgtatggctgcgacctggggccc |
| | gacgggcgcctcctccgcgggtatgaccagtccgcctacgacggcaaggattacatcgccctg |
| | aacgaggacctgcgctcctggaccgccgcggacacggcggctcagatcacccagcgcaagtgg |
| | gaggcggcccgtgaggcggagcagtggagagcctacctggagggcacgtgcgtggagtggctc |
| | cgcagatacctggagaacgggaaggagacactgcagcgcgcggaacacccaaagacacacgtg |
| | acccaccatccagtctctgaccatgaggccaccctgaggtgctgggccctgggcttctaccct |
| | gcggagatcacactgacctggcagcgggatggcgaggaccaaactcaggacaccgagcttgtg |
| | gagaccaggccagcaggagatggaaccttccagaagtgggcagctgtggtggtgccttctgga |
| | gaagagcagagatacacgtgccatgtgcagcacgaggggctgccagagcccctcaccctgaga |
| | tgggagccatcttcc |
| 481 | Tgctcccactccatgaggtatttcgacaccgccgtgtcccggcccggccgcggagagccccgc |
| | ttcatctcagtgggctacgtggacgacacgcagttcgtgcggttcgacagcgacgccgcgagt |
| | ccgagaggggagccgcgggcgccgtgggtggagcaggaggggccggagtattgggaccgggag |
| | acacagaactacaagcgccaggcacaggctgaccgagtgagcctgcggaacctgcgcggcgcc |
| | tacaaccagagcgaggacgggtctcacaccctccagaggatgtatggctgcgacctggggccc |
| | gacgggcgcctcctccgcgggtatgaccagtccgcctacgacggcaaggattacatcgccctg |
| | aacgaggacctgcgctcctggaccgccgcggacaccgcggctcagatcacccagcgcaagttg |
| | gaggcggcccgtgcggcggagcagctgagagcctacctggagggcacgtgcgtggagtggctc |
| | cgcagatacctggagaacgggaaggagacactgcagcgcgcagaacccccaaagacacacgtg |
| | acccaccaccccctctctgaccatgaggccaccctgaggtgctgggccctgggcttctaccct |
| | gcggagatcacactgacctggcagcgggatggggaggaccagacccaggacaccgagcttgtg |
| | gagaccaggccagcaggagatggaaccttccagaagtgggcagctgtggtggtgccttctgga |
| | caagagcagagatacacgtgccatatgcagcacgaggggctgcaagagcccctcaccctgagc |
| | tgggagccatcttcc |
| 482 | tgctcccactccatgaggtatttcgacaccgccgtgtcccggcccggccgcggagagccccgc |
| | ttcatctcagtgggctacgtggacgacacgcagttcgtgcggttcgacagcgacgccgcgagt |
| | ccgagaggggagccgcgggcgccgtgggtggagcaggaggggccggagtattgggaccgggag |
| | acacagaagtacaagcgccaggcacaggctgaccgagtgagcctgcggaacctgcgcggcgcc |
| | tacaaccagagcgaggacgggtctcacaccctccagaggatgtctggctgcgacctggggccc |
| | gacgggcgcctcctccgcgggtatgaccagtccgcctacgacggcaaggattacatcgccctg |
| | aacgaggacctgcgctcctggaccgccgcggacaccgcggctcagatcacccagcgcaagttg |
| | gaggcggcccgtgcggcggagcagctgagagcctacctggagggcacgtgcgtggagtggctc |
| | cgcagatacctggagaacgggaaggagacactgcagcgcgcagaacccccaaagacacacgtg |
| | acccaccaccccctctctgaccatgaggccaccctgaggtgctgggccctgggcttctaccct |
| | gcggagatcacactgacctggcagcgggatggggaggaccagacccaggacaccgagcttgtg |
| | gagaccaggccagcaggagatggaaccttccagaagtgggcagctgtggtggtgccttctgga |
| | caagagcagagatacacgtgccatatgcagcacgaggggctgcaagagcccctcaccctgagc |
| | tgggagccatcttcc |
| 483 | tgctcccactccatgaggtatttctacaccgccgtgtcccggcccggccgcggagagccccgc |
| | ttcatcgcagtgggctacgtggacgacacgcagttcgtgcggttcgacagcgacgccgcgagt |
| | ccaagaggggagccgcgggcgccgtgggtggagcaggaggggccggagtattgggaccgggag |
| | acacagaagtacaagcgccaggcacagactgaccgagtgagcctgcggaacctgcgcggcgcc |
| | tacaaccagagcgaggccgggtctcacaccctccagtggatgtatggctgcgacctggggccc |
| | gacgggcgcctcctccgcgggtatgaccagtccgcctacgacggcaaggattacatcgccctg |
| | aacgaggacctgcgctcctggaccgccgcggacacggcggctcagatcacccagcgcaagtgg |
| | gaggcggcccgtgcggcggagcagcagagagcctacctggagggcacgtgcgtggagtggctc |
| | cgcagatacctggagaacgggaaggagacactgcagcgcgcggaacacccaaagacacacgtg |
| | acccaccatttggtctctgaccatgaggccaccctgaggtgctgggccctgggcttctaccct |
| | gcggagatcacactgacctggcagcgggatggcgaggaccaaactcaggacaccgagcttgtg |
| | gagaccaggccagcaggagatggaaccttccagaagtgggcagctgtggtggtgccttctgga |
| | gaagagcagagatacacgtgccatgtgcagcacgaggggctgccggagcccctcaccctgaga |
| | tgggagccatcttcc |
| 484 | ggctcccactccttgaagtatttccacacttccgtgtcccggcccggccgcggggagccccgc |
| | ttcatctctgtgggctacgtggacgacacccagttcgtgcgcttcgacaacgacgccgcgagt |
| | ccgaggatggtgccgcgggcgccgtggatggagcaggaggggtcagagtattgggaccgggag |
| | acacggagcgccagggacaccgcacagattttccgagtgaacctgcggacgctgcgcggcgcc |
| | tacaatcagagcgaggccgggtctcacaccctgcagtggatgcatggctgcgagctggggccc |
| | gacgggcgcttcctccgcgggtatgaacagttcgcctacgacggcaaggattatctcaccctg |
| | aatgaggacctgcgctcctggaccgcggtggacacggcggctcagatctccgagcaaaagtca |
| | aatgatgcctctgaggcggagcaccagagagcctacctggaagacacatgcgtggagtggctc |
| | cacaaatacctggagaaggggaaggagacactgcttcacctggagcccccaaagacacacgtg |
| | actcaccaccccatctctgaccatgaggccaccctgaggtgctgggccctgggcttctaccct |
| | gcggagatcacactgacctggcagcaggatggggagggccatacccaggacacggagctcgtg |
| | gagaccaggcctgcaggggatggaaccttccagaagtgggcagctgtggtggtgccttctgga |
| | gaggagcagagatacacgtgccatgtgcagcatgaggggctacccgagcccgtcaccctgaga |
| | tggaagccggcttcc |

In some embodiments, the plurality of the HLA-antigen polypeptide complexes of the randomized peptide antigen libraries comprise at least about 10⁵ different HLA-antigen polypeptide complexes. Components of the 10⁵ different HLA-antigen polypeptide complexes include, collectively, at least about 10⁵ different randomized antigen polypeptides. In some embodiments, the plurality of the HLA-antigen polypeptide complexes of the randomized peptide antigen libraries comprise at least about 10⁷ different HLA-antigen polypeptide complexes. Components of the 10⁷ different HLA-antigen polypeptide complexes include, collectively, at least about 10⁷ different randomized antigen polypeptides. In some embodiments, the plurality of the HLA-antigen polypeptide complexes of the randomized peptide antigen libraries comprise at least about 10⁹ different HLA-antigen polypeptide complexes. Components of the 10⁹ different HLA-antigen polypeptide complexes include, collectively, at least about 10⁹ different randomized antigen polypeptides. In some embodiments, the plurality of the HLA-antigen polypeptide complexes of the randomized peptide antigen libraries comprise at least about 10¹¹ different HLA-antigen polypeptide complexes. Components of the 10¹¹ different HLA-antigen polypeptide complexes include, collectively, at least about 10¹¹ different randomized antigen polypeptides.

In some embodiments, the plurality of the HLA-antigen polypeptide complexes of the randomized peptide antigen libraries further comprise a β2-microglobulin polypeptide, which interacts with and stabilizes the HLA-antigen polypeptide complexes on the surface of the cell. The amino acid sequence of human β2-microglobulin polypeptide is set forth in NCBI Seq. Ref. NP_004039. In some embodiments, the human β2-microglobulin polypeptide amino acid sequence of the present disclosure is a functional naturally occurring variant of the human β2-microglobulin polypeptide having an amino acid sequence at least about 90%, 95%, 97%, 98%, or 99% identical to the human β2-microglobulin polypeptide disclosed as NCBI Seq. Ref NP_004039.

The present disclosure also includes antigen screening libraries of a plurality HLA-antigen polypeptide where the β2-microglobulin is constitutively expressed by a cell. In some embodiments, the β2-microglobulin is encoded by a first nucleic acid, the randomized antigen polypeptide encoded by a second nucleic acid, and the HLA polypeptide is encoded by a third nucleic acid. In other embodiments, the β2-microglobulin is encoded by a first nucleic acid and the randomized antigen polypeptide and the HLA polypeptide is encoded by a second nucleic acid. When encoded by the first nucleic acid, the β2-microglobulin can be transduced, transfected, or transformed into a cell before or after the second nucleic acid or the third nucleic acid.

In some embodiments of the present disclosure, the β2-microglobulin is fused to at least one of the randomized antigen polypeptides of the antigen screening library using techniques known to those of ordinary skill in the art. In these embodiments, the HLA polypeptides may or may not be a component of the antigen screening library. In other embodiments of the present disclosure, at least one of the HLA polypeptides is fused to at least one of the randomized antigen polypeptides of the antigen screening library using techniques known to those of ordinary skill in the art. In these embodiments, the β2-microglobulin can be expressed by a cell that is transduced, transfected, or transformed to express other components of the antigen screening library, such as the randomized antigen polypeptides and the HLA polypeptides. Similar to other embodiments described herein, the β2-microglobulin is constitutively expressed by the cell. In certain of these embodiments, the cell is a yeast cell. In other embodiments, the β2-microglobulin is not expressed by the cell that is transduced, transfected, or transformed to express other components of the antigen screening library, such as the randomized antigen polypeptides and the HLA polypeptides. In certain of these embodiments, the cell is a mammalian cell.

In addition to the (a) randomized antigen polypeptide, (b) MHC I HLA molecule, and (c) β2-microglobulin features of the HLA-antigen polypeptide complex of the randomized peptide antigen libraries, the HLA-antigen polypeptide complexes of the present disclosure can further include (d) a signal sequence, (e) polypeptide linkers between any or all of (a), (b), or (c), (f) a membrane tethering domain, and, optionally, (g) an epitope tag, such as a FLAG tag, a c-Myc tag, a His-tag, a hemagglutinin (HA) tag, a VSVg tag, a V5 tag, an AU1 tag, an AU5 tag, a Glu-Glu tag, an OLLAS tag, a T7 tag, an S-TagHSV tag, a KT3 tag, a TK15 tag, an Fc tag, an Xpress tag, a Ty tag, a Strep tag, an NE tag, an E tag, a C-tag, and/or an AviTag. In some embodiments, the HLA-antigen complexes do not comprise an epitope tag. However, in some embodiments, at least one or more of each of the plurality of HLA-antigen complexes of the randomized peptide antigen libraries comprise the epitope tag which allows for confirmation of expression of at least one of the HLA-antigen complexes using an antibody specific for the epitope. In some embodiments, each of the plurality of HLA-antigen complexes of the randomized peptide antigen libraries comprise the epitope tag.

In some embodiments, the membrane tethering domain comprises a polypeptide linker separating the membrane tethering domain from one or more other features ((a)-(e) and (g)) of the HLA-antigen polypeptide complex. In some embodiments, the features ((a)-(g)) of the HLA-antigen polypeptide complex are expressed as a single polypeptide. In some embodiments, the (b) HLA molecule (e.g., HLA polypeptide), the (a) randomized antigen polypeptide, and the (c) β2-microglobulin polypeptide comprise a single polypeptide. In some embodiments, the (b) HLA polypeptide and the (a) randomized antigen polypeptide are expressed as a single polypeptide, while, the (c) β2-microglobulin is expressed separately. For example, the (c) β2-microglobulin can be supplied from a separate polypeptide encoded by the same nucleic acid that expresses the (a) randomized antigen polypeptide and the (b) HLA polypeptide, a separate nucleic acid, or endogenously produced by the cell. In some embodiments, the randomized antigen polypeptide is N-terminal to the HLA polypeptide, and the HLA polypeptide is N-terminal to the β2-microglobulin polypeptide. In some embodiments, the randomized antigen polypeptide is C-terminal to the HLA polypeptide, and the HLA polypeptide is N-terminal to the β2-microglobulin polypeptide. In some embodiments, the randomized antigen polypeptide is N-terminal to the HLA polypeptide, and the HLA polypeptide is C-terminal to the β2-microglobulin polypeptide. In some embodiments, the randomized antigen polypeptide is C-terminal to the HLA polypeptide, and the HLA polypeptide is C-terminal to the β2-microglobulin polypeptide.

The (a) a randomized antigen polypeptide, (b) a major histocompatibility class I (MHC I) HLA molecule, and (c) a β2-microglobulin, can be separated by at least one flexible polypeptide linker, such as a first flexible polypeptide linker, a second flexible polypeptide linker, a third flexible polypeptide linker, a fourth flexible polypeptide linker, a fifth flexible polypeptide linker, or more flexible polypeptide linkers. In some embodiments, the at least one flexible polypeptide linker can range between about 3 and about 100 amino acid residues in length, between about 5 and about 80 amino acid residues in length, between about 10 and about 70 amino acid residues in length, between about 3 and about 100 amino acid residues in length, between about 20 and about 60 amino acid residues in length. In some embodiments, the linker can be about 5, 10, 15, 20, 25, 30, 35, 40, 45, 50, 55, 60, 65, 70, 75, 80, 85, 90, 95, or 100 amino acid residues in length. In some embodiments, the linker can be a glycine linker, or a Gly-Ser linker of the formula (GGGGS)_{X}, wherein X is 1, 2, 3, 4, 5, 6, 7, 8, 9 or 10. In some embodiments, the linker can suitably comprise a protease cleavage site such as a thrombin cleavage site.

In some embodiments, the HLA-antigen polypeptide complexes of the randomized peptide antigen libraries comprise a signal polypeptide which directs the HLA-antigen polypeptide complex to the cell surface via the secretory pathway. This signal peptide is cleaved in the endoplasmic reticulum and is not expressed by the HLA-antigen polypeptide complex when located on the cell-surface. The signal sequence can be any suitable sequence such as an endogenous HLA leader sequence, or a heterologous leader sequence imported from a different secretory or transmembrane molecule, such as an immunoglobulin leader sequence.

The HLA-antigen polypeptide complexes further comprise a membrane tethering domain, such as an anchor domain from a glycosylphosphatidylinositol (GPI) protein and/or a domain from yeast proteins having internal repeats (PIR protein). This membrane tethering domain can comprise a transmembrane domain or a domain that interacts with a cell surface protein. In some embodiments, the membrane tethering domain comprise at least one anchor domain of a GPI protein selected from the group consisting of yeast Aga2, Cwp1p, Cwp2p, Aga1p, Tip1p, Flo1p, Sed1p, YCR89w, and Tir1p and/or a PIR protein selected from the group consisting of yeast Pir1p, Pir2p, Pir3p, Pir4p, and Pir5p. A non-limiting example of membrane domain tethering is provided in FIG. 1B.

In other embodiments, components of the antigen screening libraries of a plurality HLA-antigen polypeptide complexes are expressed as more than one polypeptide and include a cleavage sequence which separates components of the antigen screening libraries of a plurality HLA-antigen polypeptide complexes from one another. For example, the randomized peptide antigen is separated from the HLA polypeptide and/or from the Beta-2 (β2) microglobulin polypeptide by the cleavage sequence. As another example, the HLA peptide is separated from the Beta-2 (β2) microglobulin polypeptide by the nucleotide encoded cleavage sequence. In some embodiments, the components of the antigen screening libraries are separated by more than one cleavage sequence. Suitable cleavage sequences are known to those of ordinary skill in the art and include, but are not limited to, self-cleaving peptides (P2A, T2A, F2A, and E2A), proteolytic cleavage sites (a 3C site, a thrombin site, a TEV site, a Factor Xa site, and an EKT site) and an internal ribosome entry sequence (IRES).

In some embodiments, the antigen screening library and/or the HLA-antigen polypeptide complexes can be expressed by one or more cells that can easily be transfected, transduced, electroporated, or transformed with the nucleic acids described herein. In some embodiments, the antigen screening library and/or the HLA-antigen polypeptide complexes are expressed on a plurality of cells. In some embodiments, each cell of the plurality of cells expresses a specific HLA-antigen complex of the HLA-antigen polypeptide complexes and/or another component of the antigen screening library. In some embodiments, a nucleic acid or a plurality of nucleic acids encode the antigen screening library and/or the HLA-antigen polypeptide complexes. In some embodiments, the antigen screening library and/or the HLA-antigen polypeptide complexes comprise prokaryotic cells. In some embodiments, the cell expressing the HLA-antigen polypeptide complexes comprise eukaryotic cells. In some embodiments, the eukaryotic cells comprise yeast cells. In some embodiments, the yeast cells are a cell of Saccharomyces cerevisiae. In some embodiments, the Saccharomyces cerevisiae is of the strain EBY100. Transforming Saccharomyces cerevisiae with nucleic acids can be achieved by standard methods as long as the efficiency is sufficient to produce at least 10⁷, 10⁸, 10⁹, or 10¹⁰ transformants.

In addition to the plurality of HLA-antigen polypeptide complexes of the antigen screening libraries described above, the present technology also includes at least two or more antigen screening libraries having HLA-antigen polypeptide complexes that differ from those described above. In some embodiments, the HLA-antigen polypeptide complexes have fewer components and/or at least one different component than the plurality of HLA-antigen polypeptide complexes described above. For example, in some embodiments, HLA-antigen polypeptide complexes can also comprise (a) an HLA polypeptide having a peptide binding cleft; and (b) a randomized antigen polypeptide comprising an amino acid sequence set forth in any one of SEQ ID NOs: 1 to 209 that specifically binds to the peptide binding cleft of the HLA polypeptide. In these embodiments, the HLA polypeptide, and the randomized antigen polypeptide comprise a single polypeptide. Also in these embodiments, the single polypeptide further comprises a first flexible polypeptide linker separating the HLA polypeptide from the randomized antigen polypeptide. When expressed on a single polypeptide separated by the first flexible polypeptide linker, the randomized antigen polypeptide is N-terminal to the HLA polypeptide on the single polypeptide or the randomized antigen polypeptide is C-terminal to the HLA polypeptide on the single polypeptide.

As another example, in some embodiments, antigen screening libraries of the present technology comprise (a) an HLA polypeptide constitutively expressed by one or more yeast cells, the HLA polypeptide comprising a peptide binding cleft, and (b) a plurality of Beta-2 (β2) microglobulin polypeptide-antigen polypeptide complexes. In these embodiments, the plurality of Beta-2 (β2) microglobulin polypeptide complexes include a randomized antigen polypeptide comprising an amino acid sequence set forth in any one of SEQ ID NOs: 1 to 209, wherein the randomized antigen polypeptide specifically binds to the peptide binding cleft of the HLA polypeptide; and (c) a Beta-2 (β2) microglobulin polypeptide. In these embodiments, the randomized antigen polypeptide and the β2-microglobulin polypeptide comprise a single polypeptide. Also, in these embodiments, the single polypeptide further comprises a first flexible polypeptide linker separating the Beta-2 (β2) microglobulin polypeptide from the randomized antigen polypeptide. When expressed on a single polypeptide separated by the first flexible polypeptide linker, the randomized antigen polypeptide is N-terminal to the Beta-2 (β2) microglobulin polypeptide on the single polypeptide or the randomized antigen polypeptide is C-terminal to the Beta-2 (β2) microglobulin polypeptide on the single polypeptide.

### Nucleic Acids Encoding HLA-Antigen Polypeptide Complexes

Also disclosed herein are nucleic acids that encode HLA-antigen polypeptide complexes of the antigen screening libraries. Nucleic acids that encode the HLA-antigen polypeptide complexes of the current disclosure minimally encode: (a) a randomized antigen polypeptide, (b) an MHC I HLA molecule, and a (c) β2-microglobulin. In addition to the (a) randomized antigen polypeptide, (b) MHC I HLA molecule, and (c) β2-microglobulin features of the HLA-antigen polypeptide complex of the randomized peptide antigen libraries encoded by one or more nucleic acids, the HLA-antigen polypeptide complexes of the present disclosure further include nucleic acids which encode (d) a signal sequence, (e) polypeptide linkers between any or all of (a), (b), or (c), (f) a membrane tethering domain, and, optionally, (g) an epitope tag, such as a FLAG tag, a c-MYC tag, a HIS-tag, a hemagglutinin tag, a VSVg tag, a V5 tag, an AU1 tag, an AU5 tag, a Glu-Glu tag, an OLLAS tag, a T7 tag, an S-Tag, an HSV tag, a KT3 tag, a TK15 tag, an Fc tag, an Xpress tag, a Ty tag, a Strep tag, an NE tag, an E tag, a C-tag, and/or an AviTag (FIG. 1A and FIG. 1B).

In some embodiments, the nucleic acid encoding the (f) membrane tethering domain may further encode (e) one or more polypeptide linkers separating the membrane tethering domain from other features of the HLA-antigen polypeptide complex. In some embodiments, the nucleic acid encodes one or more flexible polypeptide linkers which separate the (a) HLA polypeptide from the (b) randomized antigen polypeptide and the (c) β2-microglobulin polypeptide when all three features are encoded on the single nucleic acid.

In some embodiments, the nucleic acid encoding the single polypeptide further comprises nucleotides which encode a first flexible polypeptide linker and a second flexible polypeptide linker, wherein the nucleotide sequence encoding the first flexible polypeptide linker separates the nucleotide sequence encoding the HLA polypeptide from the nucleotide sequence encoding the randomized antigen polypeptide, and the nucleotide sequence encoding the second flexible polypeptide linker separates the nucleotide sequence encoding the randomized antigen polypeptide from the nucleotide sequence encoding the β2-microglobulin polypeptide. In some embodiments, once expressed, the randomized antigen polypeptide is N-terminal to the HLA polypeptide on the single polypeptide, and the HLA polypeptide is N-terminal to the β2-microglobulin polypeptide on the single polypeptide.

In some embodiments, the nucleotide sequence encoding the first flexible polypeptide linker separates the nucleotide sequence encoding the HLA polypeptide from the nucleotide sequence encoding the randomized antigen polypeptide, and the nucleotide sequence encoding the second flexible polypeptide linker separates the nucleotide sequence encoding the HLA polypeptide from the nucleotide sequence encoding the β2-microglobulin polypeptide. In some embodiments, once expressed, the randomized antigen polypeptide is C-terminal to the HLA polypeptide on the single polypeptide, and the HLA polypeptide is N-terminal to the β2-microglobulin polypeptide on the single polypeptide.

In some embodiments, the nucleotide sequence encoding the first flexible polypeptide linker separates the nucleotide sequence encoding the HLA polypeptide from the nucleotide sequence encoding the randomized antigen polypeptide, and the nucleotide sequence encoding the second flexible polypeptide linker separates the nucleotide sequence encoding the HLA polypeptide from the nucleotide sequence encoding the β2-microglobulin polypeptide. In some embodiments, once expressed, the randomized antigen polypeptide is N-terminal to the HLA polypeptide on the single polypeptide, and the HLA polypeptide is C-terminal to the β2-microglobulin polypeptide on the single polypeptide.

In some embodiments, the nucleotide sequence encoding the first flexible polypeptide linker separates the nucleotide sequence encoding the randomized antigen polypeptide from the nucleotide sequence encoding the β2-microglobulin polypeptide, and the nucleotide sequence encoding the second flexible polypeptide linker separates the nucleotide sequence encoding the β2-microglobulin polypeptide from the nucleotide sequence encoding the HLA polypeptide. In some embodiments, once expressed the randomized antigen polypeptide is C-terminal to the HLA polypeptide on the single polypeptide, and the HLA polypeptide is C-terminal to the β2-microglobulin polypeptide on the single polypeptide.

In some embodiments, the nucleotide sequence encoding the first flexible polypeptide linker separates the nucleotide sequence encoding the HLA polypeptide from the nucleotide sequence encoding the β2-microglobulin polypeptide, and the nucleotide sequence encoding the second flexible polypeptide linker separates the nucleotide sequence encoding the randomized antigen polypeptide from the nucleotide sequence encoding the HLA polypeptide. In some embodiments, once expressed, the β2-microglobulin polypeptide is C-terminal to the HLA polypeptide on the single polypeptide, and the HLA polypeptide is N-terminal to the randomized antigen polypeptide on the single polypeptide.

In some embodiments, the nucleotide sequence encoding the first flexible polypeptide linker separates the nucleotide sequence encoding the HLA polypeptide from the nucleotide sequence encoding the randomized antigen polypeptide, and the nucleotide sequence encoding the second flexible polypeptide linker separates the nucleotide sequence encoding the randomized antigen polypeptide from the nucleotide sequence encoding the β2-microglobulin polypeptide. In some embodiments, once expressed, the randomized antigen polypeptide is C-terminal to the β2-microglobulin on the single polypeptide, and the HLA polypeptide is C-terminal to the randomized antigen polypeptide on the single polypeptide. In some embodiments, the nucleotide sequence encoding the first flexible polypeptide linker separates the nucleotide sequence encoding the β2-microglobulin polypeptide from the nucleotide sequence encoding the randomized antigen polypeptide, and the nucleotide sequence encoding the second flexible polypeptide linker separates the nucleotide sequence encoding the randomized antigen polypeptide from the nucleotide sequence encoding the HLA polypeptide.

In other embodiments, components of the antigen screening libraries of a plurality HLA-antigen polypeptide complexes are expressed as more than one polypeptide despite being encoded by a single nucleic acid. In these embodiments, a nucleotide encoded cleavage sequence separates components of the antigen screening libraries of a plurality HLA-antigen polypeptide complexes from one another. For example, once expressed, the randomized peptide antigen is separated from the HLA polypeptide and/or from the Beta-2 (β2) microglobulin polypeptide by the cleavage sequence. As another example, once expressed, the HLA peptide is separated from the Beta-2 (β2) microglobulin polypeptide by the nucleotide encoded cleavage sequence. In these embodiments, a portion of the HLA polypeptide is expressed separately from other components of the antigen screening libraries of a plurality HLA-antigen polypeptide complexes and, when expressed separately, pairs naturally with the other components of the HLA-antigen polypeptide complexes inside the cell.

In some embodiments, the randomized antigen polypeptide of the HLA-antigen complex is encoded by a nucleic acid set forth in any one of SEQ ID NOs: 210 to 411. In some embodiments, the HLA polypeptide of the HLA-antigen complex is encoded by a nucleic acid at least 70%, 75%, 80%, 85%, 87.5%, 90%, 95%, 97%, 98%, 99%, or 100% homologous to any one of SEQ ID NOs: 210 to 411. In some embodiments, the randomized antigen polypeptide of the HLA-antigen complex is encoded by a nucleic acid set forth in any one of SEQ ID NOs: 412 to 426. In some embodiments, the HLA polypeptide of the HLA-antigen complex is encoded by a nucleic acid at least 70%, 75%, 80%, 85%, 87.5%, 90%, 95%, 97%, 98%, 99%, or 100% homologous to any one of SEQ ID NOs: 280 to 308. In some embodiments, one or more of the nucleic acids such as one or more of the nucleic acids of SEQ ID NOs: 210 to 411 and 412 to 426 are expressed by a plurality of cells. In some embodiments, each cell of the plurality of cells comprises a nucleic acid encoding a HLA-antigen complex. In some embodiments, the plurality of cells are a plurality of yeast cells. In some embodiments, the plurality of yeast cells are a plurality of cells of the of the EBY100 strain of Saccharomyces cerevisiae.

Nucleic acids encoding one or more components of the HLA-antigen polypeptide complexes can be delivered to the plurality of cells with a nucleic acid or a vector, such as an exogenous nucleic acid or exogenous vector. Suitable exogenous nucleic acids and exogenous vectors include plasmids, bacterial artificial chromosomes (BACs), yeast artificial chromosomes (YACs), transposons, and viral vectors. These exogenous nucleic acids and exogenous vectors can further comprise components that allow for replication of the nucleic acids encoding one or more components of the HLA-antigen polypeptide complexes, permit antibiotic selection to allow for section of cells or other organisms expressing the nucleic acids encoding one or more components of the HLA-antigen polypeptide complexes, genes that complement yeast autotrophies to select for yeast transformants expressing the nucleic acids encoding one or more components of the HLA-antigen polypeptide complexes, promoters or enhancers for prokaryotic or eukaryotic expression of the HLA-antigen polypeptide complexes, polyadenylation sites, or marker genes that allow for visualization of transformed cells. In some embodiments, the nucleic acids that comprise a nucleic acid encoding the HLA-antigen polypeptide complexes of the current disclosure comprise an inducible promoter.

Methods of using the HLA-antigen polypeptide complexes and nucleic acids encoding such complexes minimally comprise contacting one or more cells, such as a plurality of cells, expressing the HLA antigen polypeptide complexes with a TCR and selecting for one or more cells that interact with the TCR. Selection can be performed, for example, by using the TCR in a “panning step” to capture the one or more cells expressing HLA-antigen polypeptide complexes that interact with the TCR, and washing away any non-interacting cells, such as one or more cells that do not express the HLA-antigen polypeptide complexes that do not interact with the TCR. Nucleic acids from interacting cells can be harvested and sequenced to elucidate the amino acid sequences of the randomized antigen polypeptide that interacted with the TCR. These nucleic acids can be re-transfected, transformed, or transduced into one or more different cells for another round of selection. This method can be iterated for any number of rounds of selection, such as 1, 2, 3, 4, 5, or more times (e.g., in cycles) to enrich for HLA-antigen polypeptide complexes that strongly interact with the TCR.

Sequencing platforms that can be used in the present disclosure include, but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies 2005 and in U.S. Pat. Nos. 7,244,559; 7,335,762; 7,211,390; 7,244,567; 7,264,929; and 7,323,305; 2) Helicos Biosciences Corporation (Cambridge, Mass.) as described in U.S. Pat. Nos. 7,501,245; 7,491,498; and 7,276,720; and in U.S. Patent Publ. Nos. 2006/0024711; 2009/0061439; 2008/0087826; 2006/0286566; 2006/0024711; 2006/0024678; 2008/0213770; and 2008/0103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; and 7,313,308; and in US Patent Publ. Nos. 2009/0029385; 2009/0068655; 2009/0024331; and 2008/0206764.

### Methods

Described herein are methods of using the HLA-antigen polypeptide complexes of the present disclosure to select or enrich for antigens that bind to a TCR, such as a specific TCR. In some embodiments, the method includes selecting an antigen comprising contacting one or more cells, such as a plurality of cells, expressing at the HLA antigen polypeptide complexes with a TCR using one or more transgenic HLA-antigen polypeptide cell libraries, such as a transgenic HLA-antigen polypeptide yeast cell libraries. The methods described herein include methods for constructing one or more transgenic HLA-antigen polypeptide yeast cell libraries.

After construction of the one or more transgenic HLA-antigen polypeptide yeast cell libraries, the methods further include validating one or more transgenic HLA-antigen polypeptide yeast cell libraries using limiting dilution methods which include limited dilution of one or more cultures of proliferating yeast cells that each express at least one of the HLA-antigen polypeptides with nutrient-deficient yeast media. In some embodiments, the methods further include counting yeast from diluted yeast cultures and estimating HLA-antigen polypeptide yeast cell libraries with diversities of at least about 10⁶, 10⁷, 10⁸, or 10⁹ unique HLA-antigen polypeptide sequences (e.g., clones). In some embodiments, expression of an epitope tag by a yeast cell is measured to determine if any of the 10⁶, 10⁷, 10⁸, or 10⁹ clones are displayed on a yeast cell surface. For example, expression of the epitope tag can be determined as a surrogate value for total HLA-antigen polypeptide expression in the plurality of yeast cells and percent expression can be calculated. In some embodiments, the percent expression is an estimate of a number of yeast cells expressing a certain HLA-antigen polypeptide relative to the HLA-antigen polypeptide sequence library.

Referring to FIG. 2, the plurality of cells 201, such as yeast, can be transformed, transfected, or electroplated with the plurality of nucleic acids encoding the HLA-antigen polypeptide complexes of the present disclosure 202. The plurality of cells expressing the plurality of nucleic acids encoding the HLA-antigen peptide complexes is referred to as a transgenic HLA-antigen polypeptide cell library 203. The transgenic HLA-antigen polypeptide cell library 203 is expanded through cell proliferation and expression of HLA-antigen polypeptide complexes 204 by the plurality of cells is induced by methods known in the art, for example, by galactose, lactose, or isopropyl β-D-1-thiogalactopyranoside (IPTG). Cells expressing an HLA-antigen polypeptide complex that interact with a TCR are positively selected using that TCR 205. In some embodiments, the TCR is immobilized on a substrate. In some embodiments, the TCR is expressed by a cell or a plurality of cells. This selection process illustrated in FIG. 2 can be repeated for any number of rounds of selection, such as 1, 2, 3, 4, 5, or more times to arrive at a single or small number of HLA-antigen polypeptide complexes that interact with the TCR. In some embodiments, the HLA-antigen polypeptide complexes include a polypeptide antigen. In some embodiments, the polypeptide antigen is a non-naturally occurring polypeptide antigen, such as a polypeptide antigen that does not naturally occur in a human. Deep sequencing or next-generation sequencing reactions can be performed on nucleic acids extracted from the selected cells 205 after each round of selection, or after the last round of selection.

In some embodiments, greater than at least 1×10⁴, at least 1×10⁵, at least 1×10⁶, at least 1×10⁷, at least 1×10⁸, at least 1×10⁹, at least 1×10¹⁰, at least 1×10¹¹, at least 1×10¹², at least 1×10¹³, at least 1×10¹⁴, or at least 1×10¹⁵ different HLA-antigen polypeptide complexes are screened with methods of the present disclosure, such as those illustrated in FIG. 2. In some embodiments, the methods of the present disclosure result in identification of less than 10⁴, 10³, 10², 10, 9, 8, 7, 6, 5, 4, 3, or 2 different HLA-antigen polypeptide complexes. In some embodiments, greater than 90%, 95%, 97%, 98%, or 99% of the HLA-antigen polypeptide complexes remaining after 1, 2, 3, 4, or 5 rounds of selection comprise less than 10, 9, 8, 7, 6, 5, 4, 3, or 2 different HLA-antigen polypeptide complexes. In some embodiments, greater than 90%, 95%, 97%, 98%, or 99% of the HLA-antigen polypeptide complexes remaining after 1, 2, 3, 4, or 5 rounds of selection comprise less than 10, 9, 8, 7, 6, 5, 4, 3, or 2 different antigenic polypeptide sequences within the HLA-antigen polypeptide complexes. In some embodiments, greater than 90%, 95%, 97%, 98%, or 99% of the HLA-antigen polypeptide complexes remaining after 1, 2, 3, 4, or 5 rounds of selection comprise a single HLA-antigen polypeptide complex. In some embodiments, greater than 90%, 95%, 97%, 98%, or 99% of the HLA-antigen polypeptide complexes remaining after 1, 2, 3, 4, or 5 rounds of selection comprise a single antigenic polypeptide sequence within a single HLA-antigen polypeptide complex.

Expression of naive yeast libraries, such as the HLA-antigen polypeptide sequence libraries described herein, minimally express at about 15% of total antigen polypeptide sequences in an antigen polypeptide sequence library for a single length 9mer peptide presented by HLA-A1 (Gee 2018b) and less than about 5% of a single length peptide (e.g., 8mer) expression in an antigen polypeptide sequence library having mixed length peptides (e.g., 8mer, 9mer, 10 mer, 11 mer, 12mer) presented by HLA-A2 (Gee 2018a). Despite less than about 5% single length expression of the antigen polypeptide sequence library having 8mer length peptides, TCRs isolated target 8mer antigens from the antigen polypeptide sequence library that stimulated the TCR in an in vitro co-culture assay (Gee 2018a). These antigen polypeptide sequence libraries have been screened and isolate peptides against TCRs of known specificity (Gee 2018a). While a minimum level of expression necessary for a functional library has not yet been determined, data shows that less than 15% expression can result in an antigen polypeptide sequence library useful with the methods described herein.

In some embodiments, methods of the present disclosure further include identifying a polypeptide antigen that interact with a TCR. For example, a method for determining TCR interacting polypeptide antigens can comprise any of the following steps:
1. Generation and HLA-antigen polypeptide complex construct design: In some embodiments, step (1) includes, but is not limited to, generating one or more DNA constructs and/or designs to display one or more HLA polypeptides with a naturally occurring protein sequence, a synthetic protein sequences, or a combination thereof.
2. Test expression of the HLA-antigen polypeptide complex construct via yeast expression: In some embodiments, step (2) includes, but is not limited to, transforming one or more electro- or chemically competent yeast with a plasmid encoding a single peptide or library of peptides including the HLA of interest, such as the HLA polypeptide. The plasmid is designed for the single peptide construct or library of peptides constructs to display from the N-terminus of Aga2, a yeast protein. In some embodiments, expression confirmation can include antibody staining of an epitope tag (e.g., V5, VSVg, c-Myc, HA) or fluorescent TCR tetramer, dimer, or dextramer staining of yeast displaying a single peptide-HLA construct or library of peptide-HLA constructs.
3. Optional validation step for HLA display: In some embodiments, step (3) includes, but is not limited to, antibody-based staining of the epitope tag or fluorescent TCR tetramer, dimer, or dextramer staining of yeast displaying a single peptide-HLA construct or library of peptide-HLA constructs of step (2). In some embodiments, validation can also include staining a peptide-HLA construct with a TCR of known specificity or for selecting a diverse peptide library presented by the HLA.
4. Optional step to re-engineer the HLA for display: In some embodiments, step (4) includes, but is not limited to, random mutagenesis via an error-prone polymerase followed by electroporation into chemically and/or electro-competent yeast. Yeast cells expressing the library and/or libraries of the present technology are selected with cell separation by magnetic cell sorting (MACS) or fluorescence-activated cell sorting (FACS) based on a TCR of interest. In some embodiments, isolated yeast clones are sequenced or deep-sequenced to identify any functional HLA mutants that properly display antigenic peptides of interest. Step (4) is included in some embodiments if the construct or library is improperly displayed.
5. Generation of the peptide-HLA library: In some embodiments, step (5) includes, but is not limited to, randomized encoded peptide ligands or explicitly encoded peptide ligands. For example, the randomized encoded peptide ligands or explicitly encoded peptide ligands are uniquely designed for each HLA allele based on a preference for which peptides each HLA allele can present. In some embodiments, step (5) also includes generating genetic material from one or more polymerase-chain reactions.
6. Selection of the peptide-HLA library with a TCR of interest: In some embodiments, step (6) includes, but is not limited to, iterative MACS-based or FACS-based selection. For example, the TCR of interest, or other macromolecule having one or more antigen binding domains, acts as bait and can be multimerized on magnetic beads, streptavidin, dextran, or other substrates suitable for multimerization. In some embodiments, output from one or more selection rounds includes physically isolating one or more yeast cells with the TCR. Following isolation, the yeast are propagated and re-induced for protein expression. These iterative rounds enrich for binding yeast populations.
7. Deep-sequencing and data analysis: This process can involve extracting the genetic information of the yeast library and selection, and sequencing the products to identify the nature of peptides from the selected library. These data can then be analyzed to identify potential targets of TCRs and/or fed into algorithms to make predictions about TCR specificity.

### T Cell Receptors (TCRs)

The transgenic HLA-antigen polypeptide cell libraries and antigens of the HLA-antigen polypeptide complexes described herein can be used in conjunction with a given TCR. For example, the TCR, or other macromolecule having one or more antigen binding domains, is a positive selector or bait and once bound to an antigen (e.g., HLA-antigen polypeptide complex), identifies its cognate antigen. The TCRs described herein can be native or exogenous (e.g., recombinant) and expressed by a cell, such as a primary T cell, an immortalized T cell, or a non-T cell. In some embodiments, the TCR is immobilized on a solid support such as a column, a polystyrene plate or well of a multi-well plate, or a bead. In some embodiments, the TCR is multimerized as a plurality of TCRs immobilized on a bead. For example, the TCR can be multimerized on but not limited to magnetic beads, streptavidin, or dextran.

In some embodiments, the TCR is a soluble protein comprising at least one or more binding domains of a TCR of interest, e.g. TCRα/β, TCRγ/δ. The soluble protein may be a single chain, or a heterodimer. In some embodiments, the soluble TCR is modified by the addition of a biotin acceptor peptide sequence at the C terminus of one polypeptide. After biotinylation at the acceptor peptide, the TCR can be multimerized or added to substrate by binding to biotin binding partner, e.g. avidin, streptavidin, traptavidin, neutravidin, etc. In some embodiments, the biotin binding partner can comprise a detectable label, e.g. a fluorophore, mass label, etc., or can be bound to a particle, e.g. a paramagnetic particle. Selection of ligands bound to the TCR can be performed by flow cytometry, magnetic selection, and the like as known in the art.

To the extent the foregoing materials and/or any other materials incorporated herein by reference conflict with the present disclosure, the present disclosure controls.

The following examples provide further representative embodiments of the presently disclosed technology.

### EXAMPLES

The following examples are provided to further illustrate embodiments of the present technology and are not to be interpreted as limiting the scope of the present technology. To the extent that certain embodiments or features thereof are mentioned, it is merely for purposes of illustration and, unless otherwise specified, is not intended to limit the present technology. One skilled in the art may develop equivalent means without the exercise of inventive capacity and without departing from the scope of the present technology. It will be understood that many variations can be made in the procedures herein described while remaining within the bounds of the present technology. Such variations are intended to be included within the scope of the presently disclosed technology. As such, embodiments of the presently disclosed technology are described in the following representative examples.

### Example 1: Design of an Antigen Polypeptide Library

This example describes design of the antigen libraries of the present disclosure for use with a polypeptide antigen HLA complex. An exemplary algorithm to design and select anchor residues for each HLA allele is as follows, using data of known HLA binding epitopes ligands from a website such as www.IEDB.org/:

Step 1: download list of polypeptides that bind to a given allele which may comprise several hundred peptides or several thousand peptides.

Step 2: construct a frequency matrix of residues per position of the peptide based upon the downloaded known peptides.

Step 3: select composition of “anchors” for library design by using a cutoff of the top 4 residues at each position.

### Example 2: Electroporation of pHLA Library

This example describes electroporating yeast cells with nucleic acids encoding an exemplary antigen library of the present disclosure having all HLA allotypes and using peptides of 8-11 amino acids in length (8mer-11 mer). In this example, yeast cells were electroporated with nucleic acids encoding the antigen library of HLA-antigen polypeptide complexes (pHLA library).

The electroporation methods for expression of pHLA on yeast are as follows:

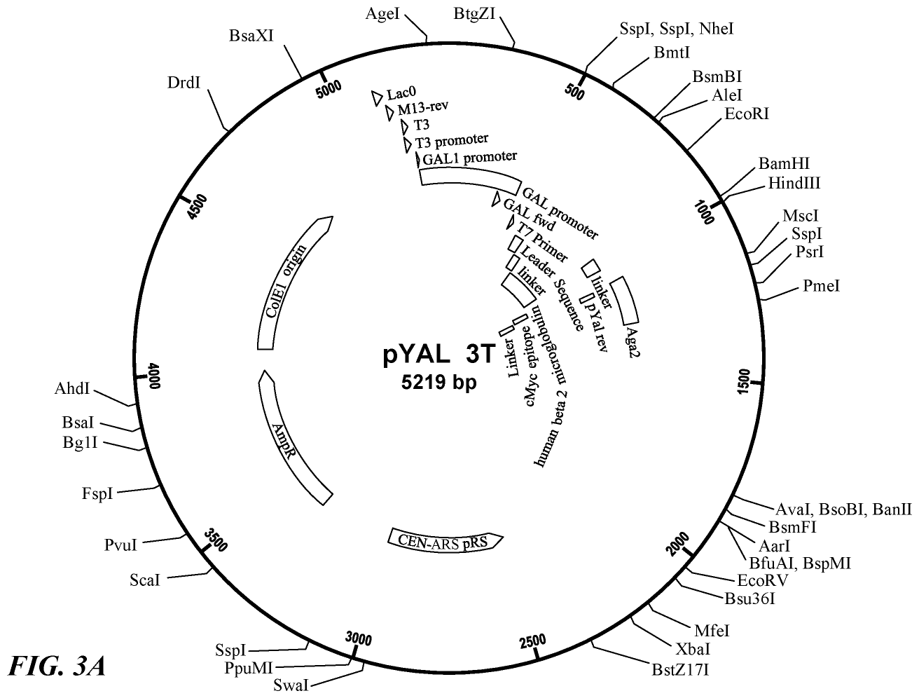

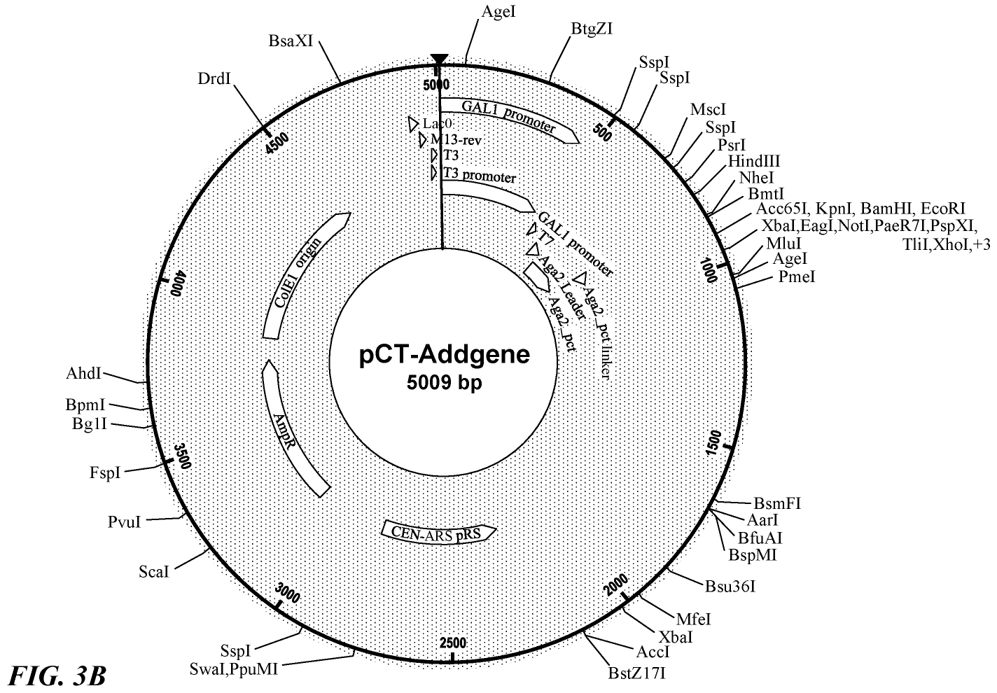

Day 0:
1. Autoclave three 2.5 L baffled flasks and one 250 ml baffled flask for expanding proliferating yeast cultures.
2. Prepare Yeast Peptone Dextrose media (YPD), which includes bacto peptone, glucose, and yeast extract.
3. Prepare two 5 ml EBY100 yeast cultures and shake at 30° C. overnight.
4. Prepare pYAL_3T plasmid (10 μg) restriction enzyme digested with HindIII, NheI or NheI, and BamHI and insert containing libraries of SEQ ID NO: 210 to 411 (50 μg). pYAL_3T vector (SEQ ID NO: 485) is a derivative of pCT vector (SEQ ID NO: 486; Invitrogen), Table 8, and the maps are provided in FIGS. 3A and 3B. Features of pYAL_3T and pCT are included in Tables 9 and 10, respectively. pYAL_3T differs from pCT at least by the orientation of the display protein scaffold (Aga2) being C-terminal of the pHLA library, the addition of human B2M, and connecting linkers. pYAL_3T has been described (Gee 2018a).

TABLE 8
Nucleotide sequences of pYAL_3T and pCT vectors

| SEQ ID NO: | Nucleotide Sequence |
|---|---|
| 485 | acggattagaagccgccgagcgggtgacagccctccgaaggaagactctcctccgtgcgt |
| | cctcgtcttcaccggtcgcgttcctgaaacgcagatgtgcctcgcgccgcactgctccga |
| | acaataaagattctacaatactagcttttatggttatgaagaggaaaaattggcagtaac |
| | ctggccccacaaaccttcaaatgaacgaatcaaattaacaaccataggatgataatgcga |
| | ttagttttttagccttatttctggggtaattaatcagcgaagcgatgatttttgatctat |
| | taacagatatataaatgcaaaaactgcataaccactttaactaatactttcaacattttc |
| | ggtttgtattacttcttattcaaatgtaataaaagtatcaacaaaaaattgttaatatac |
| | ctctatactttaacgtcaaggagaaaaaaccccggatcggactactagcagctgtaatac |
| | gactcactatagggaatattaagctaattctacttcatacattttcaattaagatgcagt |
| | tacttcgctgtttttcaatattttctgttattgctagcgttttggctggtggaggaggtt |
| | ctggaggtggtggtagtggtggtggtggttccatacaaagaactccaaagatccaagttt |
| | acagtagacatcctgctgaaaacggtaaatctaatttcttgaactgttacgtctccggtt |
| | tccacccaagtgatatagaagttgacttgttgaaaaatggtgaaagaatcgaaaaggttg |
| | aacattcagatttgtctttttctaaggactggtccttctatttgttgtactacacagaat |
| | tcactccaactgaaaaggatgaatacgcttgcagagttaatcatgtaaccttgtctcaac |
| | ctaaaatcgttaagtgggatagagacatgggtggaggtggaagtggaggtggcggttcag |
| | gtggtggcggttccggtggaggtggatccgaacaaaagcttatctccgaagaagacttgg |
| | gtggtggtggatctggtggtggtggttctggtggtggtggttctcaggaactgacaacta |
| | tatgcgagcaaatcccctcaccaactttagaatcgacgccgtactctttgtcaacgacta |
| | ctattttggccaacgggaaggcaatgcaaggagtttttgaatattacaaatcagtaacgt |
| | ttgtcagtaattgcggttctcacccctcaacaactagcaaaggcagccccataaacacac |
| | agtatgttttttgagtttaaacccgctgatctgataacaacagtgtagatgtaacaaaat |
| | cgactttgttcccactgtacttttagctcgtacaaaatacaatatacttttcatttctcc |
| | gtaaacaacatgttttcccatgtaatatccttttctatttttcgttccgttaccaacttt |
| | acacatactttatatagctattcacttctatacactaaaaaactaagacaattttaattt |
| | tgctgcctgccatatttcaatttgttataaattcctataatttatcctattagtagctaa |
| | aaaaagatgaatgtgaatcgaatcctaagagaattgggcaagtgcacaaacaatacttaa |
| | ataaatactactcagtaataacctatttcttagcatttttgacgaaatttgctattttgt |
| | tagagtcttttacaccatttgtctccacacctccgcttacatcaacaccaataacgccat |
| | ttaatctaagcgcatcaccaacattttctggcgtcagtccaccagctaacataaaatgta |
| | agctctcggggctctcttgccttccaacccagtcagaaatcgagttccaatccaaaagtt |
| | cacctgtcccacctgcttctgaatcaaacaagggaataaacgaatgaggtttctgtgaag |
| | ctgcactgagtagtatgttgcagtcttttggaaatacgagtcttttaataactggcaaac |
| | cgaggaactcttggtattcttgccacgactcatctccgtgcagttggacgatatcaatgc |
| | cgtaatcattgaccagagccaaaacatcctccttaggttgattacgaaacacgccaacca |
| | agtatttcggagtgcctgaactatttttatatgcttttacaagacttgaaattttccttg |
| | caataaccgggtcaattgttctctttctattgggcacacatataatacccagcaagtcag |
| | catcggaatctagagcacattctgcggcctctgtgctctgcaagccgcaaactttcacca |
| | atggaccagaactacctgtgaaattaataacagacatactccaagctgcctttgtgtgct |
| | taatcacgtatactcacgtgctcaatagtcaccaatgccctccctcttggccctctcctt |
| | ttcttttttcgaccgaatttcttgaagacgaaagggcctcgtgatacgcctatttttata |
| | ggttaatgtcatgataataatggtttcttaggacggatcgcttgcctgtaacttacacgc |
| | gcctcgtatcttttaatgatggaataatttgggaatttactctgtgtttatttattttta |
| | tgttttgtatttggattttagaaagtaaataaagaaggtagaagagttacggaatgaaga |
| | aaaaaaaataaacaaaggtttaaaaaatttcaacaaaaagcgtactttacatatatattt |
| | attagacaagaaaagcagattaaatagatatacattcgattaacgataagtaaaatgtaa |
| | aatcacaggattttcgtgtgtggtcttctacacagacaagatgaaacaattcggcattaa |
| | tacctgagagcaggaagagcaagataaaaggtagtatttgttggcgatccccctagagtc |
| | ttttacatcttcggaaaacaaaaactattttttctttaatttctttttttactttctatt |
| | tttaatttatatatttatattaaaaaatttaaattataattatttttatagcacgtgatg |
| | aaaaggacccaggtggcacttttcggggaaatgtgcgcggaacccctatttgtttatttt |
| | tctaaatacattcaaatatgtatccgctcatgagacaataaccctgataaatgcttcaat |
| | aatattgaaaaaggaagagtatgagtattcaacatttccgtgtcgcccttattccdtttt |
| | tgcggcattttgccttcctgtttttgctcacccagaaacgctggtgaaagtaaaagatgc |
| | tgaagatcagttgggtgcacgagtgggttacatcgaactggatctcaacagcggtaagat |
| | ccttgagagttttcgccccgaagaacgttttccaatgatgagcacttttaaagttctgct |
| | atgtggcgcggtattatcccgtgttgacgccgggcaagagcaactcggtcgccgcataca |
| | ctattctcagaatgacttggttgagtactcaccagtcacagaaaagcatcttacggatgg |
| | catgacagtaagagaattatgcagtgctgccataaccatgagtgataacactgcggccaa |
| | cttacttctgacaacgatcggaggaccgaaggagctaaccgcttttttgcacaacatggg |
| | ggatcatgtaactcgccttgatcgttgggaaccggagctgaatgaagccataccaaacga |
| | cgagcgtgacaccacgatgcctgtagcaatggcaacaacgttgcgcaaactattaactgg |
| | cgaactacttactctagcttcccggcaacaattaatagactggatggaggcggataaagt |
| | tgcaggaccacttctgcgctcggcccttccggctggctggtttattgctgataaatctgg |
| | agccggtgagcgtgggtctcgcggtatcattgcagcactggggccagatggtaagccctc |
| | ccgtatcgtagttatctacacgacgggcagtcaggcaactatggatgaacgaaatagaca |
| | gatcgctgagataggtgcctcactgattaagcattggtaactgtcagaccaagtttactc |
| | atatatactttagattgatttaaaacttcatttttaatttaaaaggatctaggtgaagat |
| | cctttttgataatctcatgaccaaaatcccttaacgtgagttttcgttccactgagcgtc |
| | agaccccgtagaaaagatcaaaggatcttcttgagatcctttttttctgcgcgtaatctg |
| | ctgcttgcaaacaaaaaaaccaccgctaccagcggtggtttgtttgccggatcaagagct |
| | accaactctttttccgaaggtaactggcttcagcagagcgcagataccaaatactgtcct |
| | tctagtgtagccgtagttaggccaccacttcaagaactctgtagcaccgcctacatacct |
| | cgctctgctaatcctgttaccagtggctgctgccagtggcgataagtcgtgtcttaccgg |
| | gttggactcaagacgatagttaccggataaggcgcagcggtcgggctgaacggggggttc |
| | gtgcacacagcccagcttggagcgaacgacctacaccgaactgagatacctacagcgtga |
| | gcattgagaaagcgccacgcttcccgaagggagaaaggcggacaggtatccggtaagcgg |
| | cagggtcggaacaggagagcgcacgagggagcttccaggggggaacgcctggtatcttta |
| | tagtcctgtcgggtttcgccacctctgacttgagcgtcgatttttgtgatgctcgtcagg |
| | ggggccgagcctatggaaaaacgccagcaacgcggcctttttacggttcctggccttttg |
| | ctggccttttgctcacatgttctttcctgcgttatcccctgattctgtggataaccgtat |
| | taccgcctttgagtgagctgataccgctcgccgcagccgaacgaccgagcgcagcgagtc |
| | agtgagcgaggaagcggaagagcgcccaatacgcaaaccgcctctccccgcgcgttggcc |
| | gattcattaatgcagctggcacgacaggtttcccgactggaaagcgggcagtgagcgcaa |
| | cgcaattaatgtgagttacctcactcattaggcaccccaggctttacactttatgcttcc |
| | ggctcctatgttgtgtggaattgtgagcggataacaatttcacacaggaaacagctatga |
| | ccatgattacgccaagctcggaattaaccctcactaaagggaacaaaagctggctagt |
| 486 | acggattagaagccgccgagcgggtgacagccctccgaaggaagactctcctccgtgcgt |
| | cctcgtcttcaccggtcgcgttcctgaaacgcagatgtgcctcgcgccgcactgctccga |
| | acaataaagattctacaatactagcttttatggttatgaagaggaaaaattggcagtaac |
| | ctggccccacaaaccttcaaatgaacgaatcaaattaacaaccataggatgataatgcga |
| | ttagttttttagccttatttctggggtaattaatcagcgaagcgatgatttttgatctat |
| | taacagatatataaatgcaaaaactgcataaccactttaactaatactttcaacattttc |
| | ggtttgtattacttcttattcaaatgtaataaaagtatcaacaaaaaattgttaatatac |
| | ctctatactttaacgtcaaggagaaaaaaccccggatcggactactagcagctgtaatac |
| | gactcactatagggaatattaagctaattctacttcatacattttcaattaagatgcagt |
| | tacttcgctgtttttcaatattttctgttattgcttcagttttagcacaggaactgacaa |
| | ctatatgcgagcaaatcccctcaccaactttagaatcgacgccgtactctttgtcaacga |
| | ctactattttggccaacgggaaggcaatgcaaggagtttttgaatattacaaatcagtaa |
| | cgtttgtcagtaattgcggttctcacccctcaacaactagcaaaggcagccccataaaca |
| | cacagtatgtttttaagcttctgcaggctagtggtggtggtggttctggtggtggtggtt |
| | ctggtggtggtggttctgctagcatgactggtggacagcaaatgggtcgggatctgtacg |
| | acgatgacgataaggtaccaggatccagtgtggtggaattctgcagatatccagcacagt |
| | ggcggccgctcgagtctagagggcccttcgaaggtaagcctatccctaaccctctcctcg |
| | gtctcgattctacgcgtaccggtcatcatcaccatcaccattgagtttaaacccgctgat |
| | ctgataacaacagtgtagatgtaacaaaatcgactttgttcccactgtacttttagctcg |
| | tacaaaatacaatatacttttcatttctccgtaaacaacatgttttcccatgtaatatcc |
| | ttttctatttttcgttccgttaccaactttacacatactttatatagctattcacttcta |
| | tacactaaaaaactaagacaattttaattttgctgcctgccatatttcaatttgttataa |
| | attcctataatttatcctattagtagctaaaaaaagatgaatgtgaatcgaatcctaaga |
| | gaattgggcaagtgcacaaacaatacttaaataaatactactcagtaataacctatttct |
| | tagcatttttgacgaaatttgctattttgttagagtcttttacaccatttgtctccacac |
| | ctccgcttacatcaacaccaataacgccatttaatctaagcgcatcaccaacattttctg |
| | gcgtcagtccaccagctaacataaaatgtaagctctcggggctctcttgccttccaaccc |
| | agtcagaaatcgagttccaatccaaaagttcacctgtcccacctgcttctgaatcaaaca |
| | agggaataaacgaatgaggtttctgtgaagctgcactgagtagtatgttgcagtcttttg |
| | gaaatacgagtcttttaataactggcaaaccgaggaactcttggtattcttgccacgact |
| | catctccgtgcagttggacgatatcaatgccgtaatcattgaccagagccaaaacatcct |
| | ccttaggttgattacgaaacacgccaaccaagtatttcggagtgcctgaactatttttat |
| | atgcttttacaagacttgaaattttccttgcaataaccgggtcaattgttctctttctat |
| | tgggcacacatataatacccagcaagtcagcatcggaatctagagcacattctgcggcct |
| | ctgtgctctgcaagccgcaaactttcaccaatggaccagaactacctgtgaaattaataa |
| | cagacatactccaagctgcctttgtgtgcttaatcacgtatactcacgtgctcaatagtc |
| | accaatgccctccctcttggccctctccttttcttttttcgaccgaatttcttgaagacg |
| | aaagggcctcgtgatacgcctatttttataggttaatgtcatgataataatggtttctta |
| | ggacggatcgcttgcctgtaacttacacgcgcctcgtatcttttaatgatggaataattt |
| | gggaatttactctgtgtttatttatttttatgttttgtatttggattttagaaagtaaat |
| | aaagaaggtagaagagttacggaatgaagaaaaaaaaataaacaaaggtttaaaaaattt |
| | caacaaaaagcgtactttacatatatatttattagacaagaaaagcagattaaatagata |
| | tacattcgattaacgataagtaaaatgtaaaatcacaggattttcgtgtgtggtcttcta |
| | cacagacaagatgaaacaattcggcattaatacctgagagcaggaagagcaagataaaag |
| | gtagtatttgttggcgatccccctagagtcttttacatcttcggaaaacaaaaactattt |
| | tttctttaatttctttttttactttctatttttaatttatatatttatattaaaaaattt |
| | aaattataattatttttatagcacgtgatgaaaaggacccaggtggcacttttcggggaa |
| | atgtgcgcggaacccctatttgtttatttttctaaatacattcaaatatgtatccgctca |
| | tgagacaataaccctgataaatgcttcaataatattgaaaaaggaagagtatgagtattc |
| | aacatttccgtgtcgcccttattcccttttttgcggcattttgccttcctgtttttgctc |
| | acccagaaacgctggtgaaagtaaaagatgctgaagatcagttgggtgcacgagtgggtt |
| | acatcgaactggatctcaacagcggtaagatccttgagagttttcgccccgaagaacgtt |
| | ttccaatgatgagcacttttaaagttctgctatgtggcgcggtattatcccgtgttgacg |
| | ccgggcaagagcaactcggtcgccgcatacactattctcagaatgacttggttgagtact |
| | caccagtcacagaaaagcatcttacggatggcatgacagtaagagaattatgcagtgctg |
| | ccataaccatgagtgataacactgcggccaacttacttctgacaacgatcggaggaccga |
| | aggagctaaccgcttttttgcacaacatgggggatcatgtaactcgccttgatcgttggg |
| | aaccggagctgaatgaagccataccaaacgacgagcgtgacaccacgatgcctgtagcaa |
| | tggcaacaacgttgcgcaaactattaactggcgaactacttactctagcttcccggcaac |
| | aattaatagactggatggaggcggataaagttgcaggaccacttctgcgctcggcccttc |
| | cggctggctggtttattgctgataaatctggagccggtgagcgtgggtctcgcggtatca |
| | ttgcagcactggggccagatggtaagccctcccgtatcgtagttatctacacgacgggca |
| | gtcaggcaactatggatgaacgaaatagacagatcgctgagataggtgcctcactgatta |
| | agcattggtaactgtcagaccaagtttactcatatatactttagattgatttaaaacttc |
| | atttttaatttaaaaggatctaggtgaagatcctttttgataatctcatgaccaaaatcc |
| | cttaacgtgagttttcgttccactgagcgtcagaccccgtagaaaagatcaaaggatctt |
| | cttgagatcctttttttctgcgcgtaatctgctgcttgcaaacaaaaaaaccaccgctac |
| | cagcggtggtttgtttgccggatcaagagctaccaactctttttccgaaggtaactggct |
| | tcagcagagcgcagataccaaatactgtccttctagtgtagccgtagttaggccaccact |
| | tcaagaactctgtagcaccgcctacatacctcgctctgctaatcctgttaccagtggctg |
| | ctgccagtggcgataagtcgtgtcttaccgggttggactcaagacgatagttaccggata |
| | aggcgcagcggtcgggctgaacggggggttcgtgcacacagcccagcttggagcgaacga |
| | cctacaccgaactgagatacctacagcgtgagcattgagaaagcgccacgcttcccgaag |
| | ggagaaaggcggacaggtatccggtaagcggcagggtcggaacaggagagcgcacgaggg |
| | agcttccaggggggaacgcctggtatctttatagtcctgtcgggtttcgccacctctgac |
| | ttgagcgtcgatttttgtgatgctcgtcaggggggccgagcctatggaaaaacgccagca |
| | acgcggcctttttacggttcctggccttttgctggccttttgctcacatgttctttcctg |
| | cgttatcccctgattctgtggataaccgtattaccgcctttgagtgagctgataccgctc |
| | gccgcagccgaacgaccgagcgcagcgagtcagtgagcgaggaagcggaagagcgcccaa |
| | tacgcaaaccgcctctccccgcgcgttggccgattcattaatgcagctggcacgacaggt |
| | ttcccgactggaaagcgggcagtgagcgcaacgcaattaatgtgagttacctcactcatt |
| | aggcaccccaggctttacactttatgcttccggctcctatgttgtgtggaattgtgagcg |
| | gataacaatttcacacaggaaacagctatgaccatgattacgccaagctcggaattaacc |
| | ctcactaaagggaacaaaagctggctagt |

TABLE 9
Features of pYAL_3T Vector

| Feature | Nucleotide Position |
|---|---|
| GAL1 promotor | 5007-450 |
| T7 | 473-494 |
| Aga2 Leader | 534-587 |
| Aga2 | 588-794 |
| Linker | 813-858 |
| CEN-ARS Prs | 2289-2799 |
| Ampr | 3129-3788 |
| Laco | 4908-4930 |

TABLE 10
Features of pCT Vector

| Feature | Position |
|---|---|
| GAL1 promotor | 5217-451 |
| T7 | 475-494 |
| Aga2 Leader | 534-587 |
| linker | 588-632 |
| hB2m | 633-929 |
| linker | 930-989 |
| epitope tag (cMyc) | 990-1019 |
| linker | 1020-1064 |
| Aga2 | 1065-1271 |
| CEN-ARS pRS | 2499-3009 |
| AmpR | 3339-3998 |
| LacO | 5118-5140 |

Day 1: Passage the two yeast cultures from Day 0 step 3 by adding 100 μl of each of the two yeast cultures to 5 ml of fresh YPD and shake at 30° C. overnight.

Day 2:
1. Measure optical density (OD) of overnight cultures from Day 1.
2. Prepare a new culture with 300 ml of an OD 0.3 yeast culture from step 1 using YPD in a 2.5 L baffled flask.
3. Prepare 3 ml of 1M Tris pH 8.0/1 M 1,4-dithiothreitol (DTT)
4. Prepare 15 ml of 2 M lithium acetate (LiAc)/10 mM Tris, 1 mM EDTA (TE)
5. Propagate culture to an OD of 1.6-2.0.
6. Add 3 ml of Tris/DTT.
7. Add 15 ml of 2 M LiAc/TE.
8. Propagate culture at 30° C. for 15 minutes while shaking at 225 rounds per minute (rpm).
9. Centrifuge culture at 3000×g for 3 minutes.
10. Resuspend the pellet in 50 mL cold E-buffer.
11. Centrifuge the suspension from step 10 at 3000×g for 3 min, at 4° C.
12. Repeat steps 10 and 11 twice.
13. Remove residual buffer.
14. Resuspend pellet in 600 μL E-buffer.
15. Add the 50 μg insert and 10 μg digested plasmid from step 4 of Day 0, total volume of buffer, insert, plasmid, and yeast should be about 1 mL.
16. Aliquot 150 μL of the suspension from step 15 into ice-chilled 2 mm gap electroporation cuvettes.
17. Electroporate each cuvette at 2.5 kV. The time constant should be between 3 and 4 ms¹.
18. Add three 1 mL volumes of cold YPD, then bring the total volume up to 200 mL with YPD
19. Culture electroporated yeast at 30° C. at 225 rpm, for 1 hour in a 250 ml baffled flask.
20. Centrifuge culture at 3500×g for 3 minutes to form a yeast cell pellet, decant the supernatant, and re-suspend the yeast cell pellet in 10 mL of SDCAA (dextrose casamino acids, which also includes, yeast nitrogen base without amino acids and with ammonium sulfate, sodium citrate, and citric acid monohydrate, at a pH of 4.5).

Day 2: Determine Titer
1. Add 990 μL of SDCAA to each of four Eppendorf tubes.
2. Add 10 μL from step 20 above to one tube containing 990 μL SDCAA.
3. Pipet 100 μL of the 10⁴ solution into a tube containing only 990 μL of SDCAA.
4. Pipet 100 μL of the 10⁵ solution into a tube containing only 990 μL of SDCAA.
5. Pipet 100 μL of the 10⁶ solution into a tube containing only 990 μL of SDCAA.
6. Spread 100 μL of each dilution in steps 2-5 onto a separate SDCAA plates and incubate at 30° C. for 3 days. Count the colonies on the plates to determine the titer. From step 2, the colonies counted represent the diversity of the library x 10⁴. From step 3, the colonies counted represent the diversity of the library x 10⁵. From step 4, the colonies counted represent the diversity of the library x 10⁶. From step 5, the colonies counted represent the diversity of the library x 10⁷.
7. Add 490 ml of pH 4.5 SDCAA to the remaining cell suspension from step 20 and culture at 30° C. overnight.

Day 3: Measure the OD of the passage from step 8 after 24 hours. The OD should be at least 5. Passage the culture to an OD of 1 in a total volume of 500 mL SDCAA.

Day 4: Passage cells to an OD of 1 in a total volume of 500 mL SDCAA.

Day 5: 72 hours after step 18 from Day 2 was performed, induce in SGCAA (galactose casamino acids, which also includes, yeast nitrogen base without amino acids and with ammonium sulfate, sodium citrate, and citric acid monohydrate, at a pH of 4.5).

Recipes:
1) E-buffer, 500 ml
   0.6 g Tris base,
   91.09 g Sorbitol (1M)
   73.50 mg CaCl2 (1 mM; consider making 1M stock solution) in ddH2O to a final volume of 500 ml, pH to 7.5. Filter through 0.22 μm membrane.
2) 1 M Tris/1 M DTT, 3 ml
   0.462 g 1,4-dithiothreitol in 3 ml 1 M Tris, pH 8.0 and sterilize by filtration.
3) 2 M LiAc/TE solution, 15 ml
   1.98 g LiAc in 10 ml of TE (10 mM Tris, 1 mM EDTA), sterilize by filtration.

### Example 3: Characterization of pHLA Expression

This example describes characterizing expression of HLA-antigen polypeptide complexes on the electroporated yeast cells of Example 2. These expression measurements include FACS analysis to determine the levels of peptide-MHC displayed on the surface of yeast cells and indicate functionality of the random yeast display library. The characterization methods for expression of pHLA on yeast are as follows:

Materials
1. Yeast library from Example 2
2. PBSM (1×PBS, 1 g/L bovine serum albumin, EDTA, pH 7.4; filtered)
3. Anti-myc (FITC fluorophore-conjugated) antibody
4. 96-well U-bottom plate

Optional:
5. Anti-VS (647 fluorophore-conjugated) antibody
6. Anti-HA (BV421 fluorophore-conjugated) antibody
7. Anti-VSV (PE fluorophore-conjugated) antibody

Cell Preparation
1. Measure optical density of yeast culture on NanoDrop. OD600 readings between 0 to 1 are in the liner range for cultures induced 2 to 3 days at 20° C. at a 1:20 culture:SDCAA dilution.
2. Transfer samples of yeast cultures into wells of a 96-well plate. For yeast cultures having an OD600 of about 10, use 254, of the culture. Include single-color and unstained controls.
3. Add PBSM to 200 μl final volume to each well.
4. Centrifuge the 96-well plate at 2500×g for 2 minutes.
5. Remove supernatants.

Staining Cells
1. Re-suspend each cell pellet in 100 μl PBSM
2. Add 1 μl antibody as appropriate. 3. Incubate at 4° C., protected from light (e.g., dark) for 30 minutes.

Washing Cells and Determining pHLA Expression
1. Centrifuge the 96-well plate at 2500×g for 2 minutes.
2. Remove supernatants.
3. Re-suspend each pellet in 200 μl PB SM.
4. Centrifuge the 96-well plate at 2500×g for 2 minutes.
5. Remove supernatants.
6. Re-suspend each pellet in 200 μl PB SM.
7. Analyze samples in each well with CytoFlex.

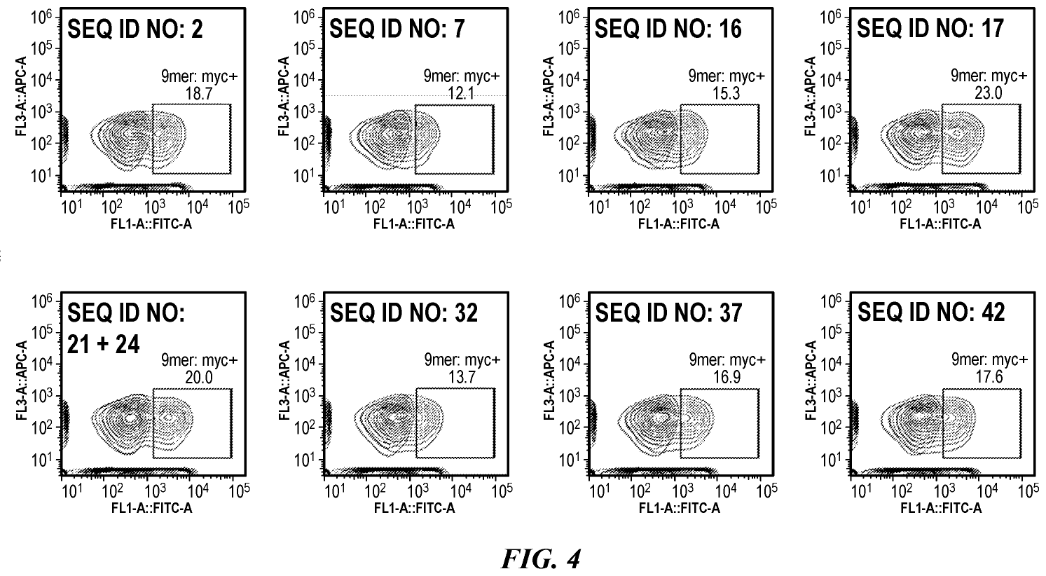

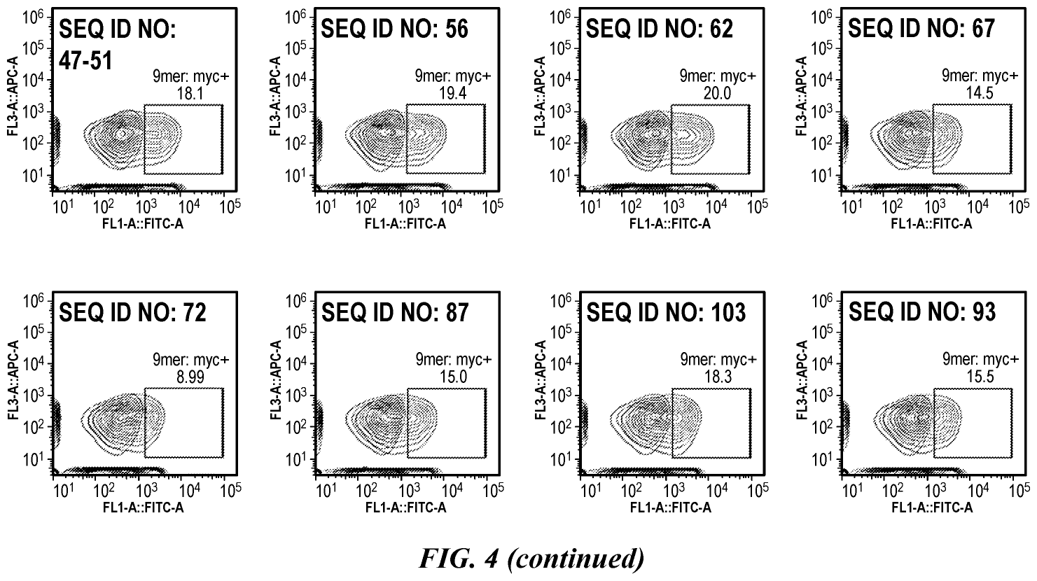

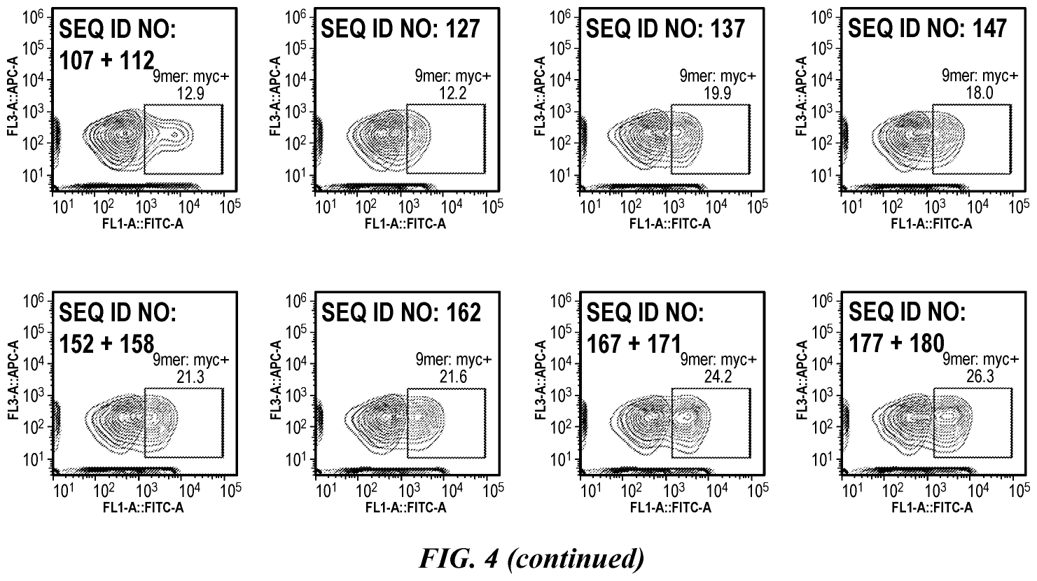

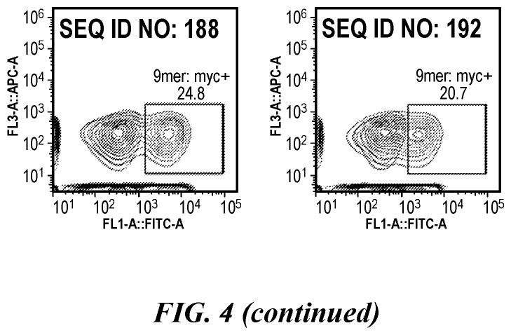

Results: Expression of the HLA-antigen polypeptide complexes (peptides of SEQ ID Nos: 8, 11, 14, 18, 21+24, 28, 32, 36, 40-44, 47, 50, 53, 56, 65, 75, 69, 77+80, 89, 95, 99, 102+106, 108, 111+114, 117+120, 124, and 125) was determined by flow cytometry and shown in FIG. 4. Antibodies targeting epitope tags expressed by HLA-antigen polypeptide complexes were used to stain electroporated yeast cells. FITC-A staining corresponds to HLA-antigen polypeptide complexes expressing a c-Myc tag. Antibody-epitope tag binding was used as a proxy to determine pHLA expression, which, as shown in FIG. 4, ranged from 8.99% for SEQ ID NO: 56 to 26.3% expression for SEQ ID NOs: 177+180.

### Example 4: Functional Validation of pHLA Expression

This example describes functionally validating expression of pHLAs on the electroporated yeast cells of Example 2 with a candidate TCR. Expected target antigens of the pHLAs can be identified from up to 6 libraries when the candidate TCR is allotype-matched. The functional validation methods for expression of pHLA on yeast are as follows:

HLA-antigen polypeptide sequence libraries, such as those disclosed herein, minimally express about 25% of total antigen polypeptide sequences for a single length 9mer peptide presented by HLA-A1 (Gee 2018b) and express less than about 5% of a single length peptide (e.g., 8mer) having mixed length peptides (e.g., 8mer, 9mer, 10 mer, 11 mer) presented by HLA-A2 (Gee 2018a). Despite less than about 5% single length peptide expression of the HLA-antigen polypeptide sequence having 8mer length peptides, isolated TCRs of interest target 8mer antigens from the HLA-antigen polypeptide complexes. These isolated TCR of interest were stimulated by one or more HLA-antigen polypeptide complexes in an in vitro co-culture assay (Gee 2018a; see FIGS. 5C and 7A therein). HLA-antigen polypeptide libraries have been screened and peptides which bind TCRs of known specificity have been isolated (Gee 2018a). While a minimum level of expression that is necessary for a functional HLA-antigen polypeptide library of the present disclosure has not yet been determined, data shows that less than 15% expression can result in an HLA-antigen polypeptide library that is useful with the methods described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### REFERENCES

Altschul et al. “Basic local alignment search tool.” J Mol Biol 215(3):403-410 (1990) Altschul & Karlin. “Methods for assessing the statistical significance of molecular sequence features by using general scoring schemes.” Proc Natl Acad Sci USA 87(6):2264-2268 (1990)
Altschul & Karlin. “Applications and statistics for multiple high-scoring segments in molecular sequences.” Proc Natl Acad Sci USA 90(12):5873-5877 (1993)
Arden et al. “Human T-cell receptor variable gene segment families.” Immunogenetics 42(6):455-500 (1995)
Bowie et al. “A method to identify protein sequences that fold into a known three-dimensional structure.” Science 253(5016):164-170 (1991)
Brenner et al. “Population statistics of protein structures: lessons from structural classifications.” Curr Opin Struct Biol 7(3):369-376 (1997)
Chou & Fasman. “Prediction of protein conformation.” Biochemistry 13(2):222-245 (1974a)
Chou & Fasman. “Conformational parameters for amino acids in helical, beta-sheet, and random coil regions calculated from proteins.” Biochemistry 113(2):211-222 (1974b)
Chou & Fasman. “Prediction of the secondary structure of proteins from their amino acid sequence.” Adv Enzymol Relat Areas Mol Biol 47:45-148 (1978a)
Chou & Fasman. “Empirical predictions of protein conformation.” Annu Rev Biochem 47:251-276 (1978b)
Chou & Fasman. “Prediction of beta-turns.” Biophys J 26:367-384 (1979)
Gee et al. “Antigen identification for orphan T cell receptors expressed on tumor-infiltrating lymphocytes.” Cell 172(3):549-563 (2018a)
Gee et al. “Facile method for screening clinical T cell receptors for off-target peptide-HLA reactivity.” bioRxiv 472480 (2018b)
Gribskov et al. “Profile analysis: detection of distantly related proteins.” Proc Natl Acad Sci USA 84(13):4355-4358 (1987)
Gribskov et al. Profile analysis.” Methods Enzymol 183:146-159 (1990)
Holm & Sander. “Protein folds and families: sequence and structure alignments.” Nucleic Acids Res 27(1):244-247 (1999)
Jones. “Progress in protein structure prediction.” Curr Opin Struct Biol 7(3):377-87 (1997)
Jones et. al. “Engineering and Characterization of a Stabilized α1/α2 Module of the Class I Major Histocompatibility Complex Product L.” J. of Biol. Chem. 281(35):25734-25744 (2006)
Kotsiou et al. “Properties and Applications of Single-Chain Major Histocompatibility Complex Class I Molecule.” Antioxid. Redox Signal. 15(3):645-655 (2011)
Kyte & Doolittle. “A simple method for displaying the hydropathic character of a protein.” J Mol Biol 157(1):105-132 (1982)
Mackelprang et al. “Sequence diversity, natural selection and linkage disequilibrium in the human T cell receptor alpha/delta locus.” Hum Genet 119(3):255-266 (2006)
MacLennan et al. “Structure-function relationships in the Ca(2+)-binding and translocation domain of SERCA1: physiological correlates in Brody disease.” Acta Physiol Scand Suppl 643:55-67 (1998)
Margulies et al. “Genome sequencing in microfabricated high-density picolitre reactors.” Nature 437(7057):376-380 (2005)
Mottez et al. “Cells Expressing a Major Histocompatibility Complex Class I Molecule with a Single Covalently Bound Peptide Are Highly Immunogenic.” J. Exp. Med. 181: 493-502 (1995)
Moult. “The current state of the art in protein structure prediction.” Curr Opin Biotechnol 7(4):422-427 (1996)
Pandey et al. “Current strategies for protein production and purification enabling membrane protein structural biology.” Biochem Cell Biol. 94(6) 507-527 (2016)
Rowen et al. “The complete 685-kilobase DNA sequence of the human beta T cell receptor locus.” Science 272(5269):1755-1762 (1996)
Sasaki & Sutoh. “Structure-mutation analysis of the ATPase site of Dictyostelium discoideum myosin II.” Adv Biophys 35:1-24 (1998)
Sippl & Flockner. “Threading thrills and threats.” Structure 4(1):15-19 (1996)
Tafuro et al. “Reconstitution of Antigen Presentation in HLA Class I-Negative Cancer Cells with Peptide-β2m Fusion Molecules.” Eur. J. Immunol. 31: 440-449 (2001)
White et al. “Soluble Class I MHC with β2-Microglobulin Covalently Linked Peptides: Specific Binding to a T Cell Hybridoma.” J. Immunol. 162: 2671-2676 (1999)

1. An antigen screening library comprising a plurality of Human Leukocyte Antigen (HLA)-antigen polypeptide complexes, the HLA-antigen polypeptide complexes comprising:
a) an HLA polypeptide, the HLA polypeptide comprising a peptide binding cleft;
b) a randomized antigen polypeptide comprising an amino acid sequence set forth in any one of SEQ ID NOs: 1 to 209, wherein the randomized antigen polypeptide specifically binds to the peptide binding cleft of the HLA polypeptide; and
c) a Beta-2 (β2) microglobulin polypeptide.

2. The antigen screening library of claim 1, wherein the plurality of HLA-antigen complexes comprises an HLA polypeptide selected from the list consisting of A3, A11, A23, A24, A26, A30, A31, A33, A68, B7, B8, B15, B27, B40, B44, B51, B53, B57, C1, C2, C3, C4, C5, C6, C7, C8, and E.

3. The antigen screening library of claim 1, wherein the plurality of HLA-antigen complexes comprises at least five, ten, fifteen, twenty, or twenty-five different HLA polypeptides selected from the list consisting of A3, A11, A23, A24, A26, A30, A31, A33, A68, B7, B8, B15, B27, B40, B44, B51, B53, C1, C2, C3, C4, C5, C6, C7, C8, and E.

4. The antigen screening library of claim 1, wherein the plurality of HLA-antigen complexes comprises all of A3, A11, A23, A24, A26, A30, A31, A33, A68, B7, B8, B15, B27, B40, B44, B51, B53, C1, C2, C3, C4, C5, C6, C7, C8, and E HLA polypeptides.

5. The antigen screening library of claim 1, wherein the plurality of HLA-antigen complexes comprises an HLA polypeptide comprising an amino acid sequence at least 87.5%, 90%, 95%, 97%, 98%, 99%, or 100% identical to an amino acid sequence set forth in any one of SEQ ID NOs: 427 to 455.

6. The antigen screening library of any one of claims 1 to 5, wherein the plurality of the HLA-antigen polypeptide complexes comprises at least about 10⁵ different HLA-antigen polypeptide complexes comprising at least about 10⁵ different randomized antigen polypeptides.

7. The antigen screening library of any one of claims 1 to 6, wherein the HLA polypeptide, the randomized antigen polypeptide, and the β2-microglobulin polypeptide comprise a single polypeptide.

8. The antigen screening library of claim 7, wherein the single polypeptide further comprises a first flexible polypeptide linker and a second flexible polypeptide linker.

9. The antigen screening library of claim 8, wherein the randomized antigen polypeptide is N-terminal to the HLA polypeptide on the single polypeptide, and the HLA polypeptide is N-terminal to the β2-microglobulin polypeptide on the single polypeptide.

10. The antigen screening library of claim 9, wherein the first flexible polypeptide linker separates the HLA polypeptide from the randomized antigen polypeptide, and a second flexible polypeptide linker separates the HLA polypeptide from the β2-microglobulin polypeptide.

11. The antigen screening library of claim 8, wherein the randomized antigen polypeptide is C-terminal to the HLA polypeptide on the single polypeptide, and the HLA polypeptide is N-terminal to the β2-microglobulin polypeptide on the single polypeptide.

12. The antigen screening library of claim 11, wherein the first flexible polypeptide linker separates the HLA polypeptide from the randomized antigen polypeptide, and a second flexible polypeptide linker separates the HLA polypeptide from the β2-microglobulin polypeptide.

13. The antigen screening library of claim 8, wherein the randomized antigen polypeptide is N-terminal to the HLA polypeptide on the single polypeptide, and the HLA polypeptide is C-terminal to the β2-microglobulin polypeptide on the single polypeptide.

14. The antigen screening library of claim 13, wherein the first flexible polypeptide linker separates the randomized antigen polypeptide from the β2-microglobulin polypeptide, and a second flexible polypeptide linker separates the β2-microglobulin polypeptide from the HLA polypeptide.

15. The antigen screening library of claim 8, wherein the randomized antigen polypeptide is C-terminal to the HLA polypeptide on the single polypeptide, and the HLA polypeptide is C-terminal to the β2-microglobulin polypeptide on the single polypeptide.

16. The antigen screening library of claim 15, wherein the first flexible polypeptide linker separates the HLA polypeptide from the β2-microglobulin polypeptide, and a second flexible polypeptide linker separates the randomized antigen polypeptide from the HLA polypeptide.

17. The antigen screening library of claim 8, wherein the β2-microglobulin polypeptide is C-terminal to the HLA polypeptide on the single polypeptide, and the HLA polypeptide is N-terminal to the randomized antigen polypeptide on the single polypeptide.

18. The antigen screening library of claim 17, wherein the first flexible polypeptide linker separates the HLA polypeptide from the randomized antigen polypeptide, and a second flexible polypeptide linker separates the randomized antigen polypeptide from the β2-microglobulin polypeptide.

19. The antigen screening library of claim 8, wherein the randomized antigen polypeptide is C-terminal to the β2-microglobulin on the single polypeptide, and the HLA polypeptide is C-terminal to the randomized antigen polypeptide on the single polypeptide.

20. The antigen screening library of claim 19, wherein the first flexible polypeptide linker separates the β2-microglobulin polypeptide from the randomized antigen polypeptide, and a second flexible polypeptide linker separates the randomized antigen polypeptide from the HLA polypeptide.

21. The antigen screening library of any one of claims 1 to 20, wherein each of the HLA-antigen complexes of the plurality of the HLA-antigen complexes do not comprise an epitope tag.

22. The antigen screening library of any one of claims 1 to 20, wherein at least one of the HLA-antigen complexes of the plurality of HLA-antigen complexes comprise an epitope tag.

23. The antigen screening library of any one of claims 1 to 20, wherein at least one of the HLA-antigen complexes of the plurality of HLA-antigen complexes does not comprise an epitope tag and at least one of the HLA-antigen complexes of the plurality of HLA-antigen complexes does comprise an epitope tag.

24. The antigen screening library of claim 22 or 23, wherein the epitope tag comprises a FLAG tag, a c-Myc tag, a HIS-tag, a hemagglutinin (HA) tag, a VSVg tag, or a V5 tag.

25. The antigen screening library of any one of claims 1 to 24, wherein the HLA-antigen complexes each comprise a membrane tethering domain.

26. The antigen screening library of claim 25, wherein the membrane tethering domain comprises Aga2.

27. The antigen screening library of any one of claims 1 to 26, wherein the antigen screening library is expressed on a plurality of cells.

28. The antigen screening library of claim 27, wherein the plurality of cells are a plurality of yeast cells.

29. The antigen screening library of claim 28, wherein the plurality of yeast cells are a plurality of yeast cells of the EBY100 strain of Saccharomyces cerevisiae.

30. The antigen screening library of any one of claims 27 to 29, wherein each cell of the plurality of cells expresses a specific HLA-antigen complex.

31. An antigen screening library comprising a plurality of Human Leukocyte Antigen (HLA)-antigen polypeptide complexes, the HLA-antigen polypeptide complexes comprising:
a) an HLA polypeptide, the HLA polypeptide comprising a peptide binding cleft; and
b) a randomized antigen polypeptide comprising an amino acid sequence set forth in any one of SEQ ID NOs: 1 to 209, wherein the randomized antigen polypeptide specifically binds to the peptide binding cleft of the HLA polypeptide.

32. The antigen screening library of claim 31, wherein the plurality of HLA-antigen complexes comprises an HLA polypeptide selected from the list consisting of A3, A11, A23, A24, A26, A30, A31, A33, A68, B7, B8, B15, B27, B40, B44, B51, B53, B57, C1, C2, C3, C4, C5, C6, C7, C8, and E.

33. The antigen screening library of claim 31, wherein the plurality of HLA-antigen complexes comprises at least five, ten, fifteen, twenty, or twenty-five different HLA polypeptides selected from the list consisting of A3, A11, A23, A24, A26, A30, A31, A33, A68, B7, B8, B15, B27, B40, B44, B51, B53, C1, C2, C3, C4, C5, C6, C7, C8, and E.

34. The antigen screening library of claim 31, wherein the plurality of HLA-antigen complexes comprises all of A3, A11, A23, A24, A26, A30, A31, A33, A68, B7, B8, B15, B27, B40, B44, B51, B53, C1, C2, C3, C4, C5, C6, C7, C8, and E HLA polypeptides.

35. The antigen screening library of claim 31, wherein the plurality of HLA-antigen complexes comprises an HLA polypeptide comprising an amino acid sequence at least 87.5%, 90%, 95%, 97%, 98%, 99%, or 100% identical to an amino acid sequence set forth in any one of SEQ ID NOs: 427 to 455.

36. The antigen screening library of any one of claims 31 to 35, wherein the plurality of the HLA-antigen polypeptide complexes comprises at least about 10⁵ different HLA-antigen polypeptide complexes comprising at least about 10⁵ different randomized antigen polypeptides.

37. The antigen screening library of any one of claims 31 to 36, wherein the HLA polypeptide, and the randomized antigen polypeptide comprise a single polypeptide.

38. The antigen screening library of claim 37, wherein the single polypeptide further comprises a first flexible polypeptide linker separating the HLA polypeptide from the randomized antigen polypeptide.

39. The antigen screening library of claim 38, wherein the randomized antigen polypeptide is N-terminal to the HLA polypeptide on the single polypeptide.

40. The antigen screening library of claim 38, wherein the randomized antigen polypeptide is C-terminal to the HLA polypeptide on the single polypeptide.

41. The antigen screening library of any one of claims 31 to 40, wherein each of the HLA-antigen complexes of the plurality of the HLA-antigen complexes do not comprise an epitope tag.

42. The antigen screening library of any one of claims 31 to 40, wherein at least one of the HLA-antigen complexes of the plurality of HLA-antigen complexes comprise an epitope tag.

43. The antigen screening library of any one of claims 31 to 40, wherein at least one of the HLA-antigen complexes of the plurality of HLA-antigen complexes does not comprise an epitope tag and at least one of the HLA-antigen complexes of the plurality of HLA-antigen complexes does comprise an epitope tag.

44. The antigen screening library of claim 42 or 43, wherein the epitope tag comprises a FLAG tag, a c-Myc tag, a HIS-tag, a hemagglutinin (HA) tag, a VSVg tag, or a V5 tag.

45. The antigen screening library of any one of claims 31 to 44, wherein the HLA-antigen complexes each comprise a membrane tethering domain.

46. The antigen screening library of claim 45, wherein the membrane tethering domain comprises Aga2.

47. The antigen screening library of any one of claims 31 to 46, wherein the antigen screening library is expressed on a plurality of cells.

48. The antigen screening library of claim 47, wherein the plurality of cells are a plurality of yeast cells.

49. The antigen screening library of claim 48, wherein the plurality of yeast cells are a plurality of yeast cells of the EBY100 strain of Saccharomyces cerevisiae.

50. The antigen screening library of any one of claims 47 to 49, wherein each cell of the plurality of cells expresses a specific HLA-antigen complex.

51. The antigen screening library of any one of claims 47 to 50, wherein each cell of the plurality of cells expresses a β2-microglobulin polypeptide.

52. An antigen screening library comprising
a) a plurality of antigen polypeptide-Beta-2 (β2) microglobulin polypeptide complexes, the antigen polypeptide-Beta-2 (β2) microglobulin polypeptide complexes comprising:
i) a randomized antigen polypeptide comprising an amino acid sequence set forth in any one of SEQ ID NOs: 1 to 209, wherein the randomized antigen polypeptide specifically binds to the peptide binding cleft of the HLA polypeptide; and
ii) a Beta-2 (β2) microglobulin polypeptide; and
b) a plurality of HLA polypeptides constitutively expressed by one or more yeast cells and comprising a peptide binding cleft.

53. The antigen screening library of claim 52, wherein the plurality of HLA polypeptides is selected from the list consisting of A3, A11, A23, A24, A26, A30, A31, A33, A68, B7, B8, B15, B27, B40, B44, B51, B53, B57, C1, C2, C3, C4, C5, C6, C7, C8, and E.

54. The antigen screening library of claim 52, wherein the plurality of HLA polypeptides comprises at least five, ten, fifteen, twenty, or twenty-five different HLA polypeptides selected from the list consisting of A3, A11, A23, A24, A26, A30, A31, A33, A68, B7, B8, B15, B27, B40, B44, B51, B53, C1, C2, C3, C4, C5, C6, C7, C8, and E.

55. The antigen screening library of claim 52, wherein the plurality of HLA polypeptides comprises all of A3, A11, A23, A24, A26, A30, A31, A33, A68, B7, B8, B15, B27, B40, B44, B51, B53, C1, C2, C3, C4, C5, C6, C7, C8, and E HLA polypeptides.

56. The antigen screening library of claim 52, wherein the plurality of HLA polypeptides comprises an HLA polypeptide comprising an amino acid sequence at least 87.5%, 90%, 95%, 97%, 98%, 99%, or 100% identical to an amino acid sequence set forth in any one of SEQ ID NOs: 427 to 455.

57. The antigen screening library of any one of claims 52 to 56, wherein the plurality of the antigen polypeptide-Beta-2 (β2) microglobulin polypeptide complexes comprises at least about 10⁵ different antigen polypeptide-Beta-2 (β2) microglobulin polypeptide complexes comprising at least about 10⁵ different randomized antigen polypeptides.

58. The antigen screening library of any one of claims 52 to 57, wherein the randomized antigen polypeptide and the β2-microglobulin polypeptide comprise a single polypeptide.

59. The antigen screening library of claim 58, wherein the single polypeptide further comprises a first flexible polypeptide linker.

60. The antigen screening library of claim 59, wherein the randomized antigen polypeptide is N-terminal to the β2-microglobulin polypeptide on the single polypeptide.

61. The antigen screening library of claim 60, wherein the randomized antigen polypeptide is C-terminal to the β2-microglobulin polypeptide on the single polypeptide.

62. The antigen screening library of any one of claims 52 to 61, wherein each of the antigen polypeptide-Beta-2 (β2) microglobulin polypeptide complexes of the plurality of the antigen polypeptide-Beta-2 (β2) microglobulin polypeptide complexes do not comprise an epitope tag.

63. The antigen screening library of any one of claims 52 to 61, wherein at least one of the antigen polypeptide-Beta-2 (β2) microglobulin polypeptide complexes of the plurality of antigen polypeptide-Beta-2 (β2) microglobulin polypeptide complexes comprise an epitope tag.

64. The antigen screening library of any one of claims 52 to 61, wherein at least one of the HLA-antigen complexes of the plurality of HLA-antigen complexes does not comprise an epitope tag and at least one of the HLA-antigen complexes of the plurality of HLA-antigen complexes does comprise an epitope tag.

65. The antigen screening library of claim 63 or 64, wherein the epitope tag comprises a FLAG tag, a c-Myc tag, a HIS-tag, a hemagglutinin (HA) tag, a VSVg tag, or a V5 tag.

66. The antigen screening library of any one of claims 52 to 65, wherein the antigen polypeptide-Beta-2 (β2) microglobulin polypeptide complexes each comprise a membrane tethering domain.

67. The antigen screening library of claim 66, wherein the membrane tethering domain comprises Aga2.

68. The antigen screening library of any one of claims 52 to 67, wherein the antigen screening library is expressed on a plurality of cells.

69. The antigen screening library of claim 68, wherein the plurality of cells are a plurality of yeast cells.

70. The antigen screening library of claim 69, wherein the plurality of yeast cells are a plurality of yeast cells of the EBY100 strain of Saccharomyces cerevisiae.

71. The antigen screening library of any one of claims 68 to 70, wherein each cell of the plurality of cells expresses a specific antigen polypeptide-Beta-2 (β2) microglobulin polypeptide complex.

72. A plurality of nucleic acids encoding the antigen screening library of any one of claims 1 to 71.

73. The plurality of nucleic acids of claim 72, wherein the HLA polypeptide is encoded by a nucleic acid that is at least about 85%, 87.5%, 90%, 95%, 97%, 98%, or 99% homologous to any one of SEQ ID NOs: 456 to 484.

74. The plurality of nucleic acids of claim 73, wherein the randomized antigen polypeptide is encoded by a nucleic acid set forth in any one of SEQ ID NOs: 210 to 426.

75. The plurality of nucleic acids of any one of claims 72 to 74, wherein the plurality of nucleic acids is expressed by a plurality of cells.

76. A plurality of cells expressing the antigen screening library of any one of claims 1 to 75.

77. The plurality of cells of claim 76, wherein the plurality of cells is a plurality of yeast cells.

78. The plurality of cells of claim 77, wherein the plurality of yeast cells is a plurality of cells of the EBY100 strain of Saccharomyces cerevisiae.

79. The plurality of cells of any one of claims 76 to 78, wherein each cell of the plurality of cells comprises a nucleic acid of the plurality of nucleic acids encoding a specific of HLA-antigen complex.

80. A method of selecting an antigen comprising contacting the plurality of cells of any one of claims 76 to 79 with a T cell receptor (TCR) or other macromolecule having one or more antigen binding domains.

81. The method of claim 80, wherein the TCR or other macromolecule having one or more antigen binding domains is immobilized on a substrate.

82. The method of claim 80, wherein the TCR or other macromolecule having one or more antigen binding domains is expressed by a cell.

83. The method of any one of claims 76 to 79, wherein the selection is repeated for 2, 3, 4, or 5 cycles.

84. The method of claim 83, wherein the antigen is a polypeptide antigen.

85. The method of claim 84, wherein the antigen is a polypeptide antigen that does not naturally occur.

86. The method of claim 85, wherein the antigen is a polypeptide antigen that does not naturally occur in a human.
